(19) United States
(12) Patent Application Publication
(10) Pub. No.: US 2024/0215615 A1
(43) Pub. Date: Jul. 4, 2024

(54) URIDINE DIPHOSPHATE-DEPENDENT GLYCOSYLTRANSFERASE CIRCULAR PERMUTANTS

(71) Applicant: Manus Bio Inc., Waltham, MA (US)

(72) Inventors: Ryan PHILIPPE, Waltham, MA (US); Ajikumar Parayil KUMARAN, Waltham, MA (US); Jason DONALD, Waltham, MA (US); Krishna PATEL, Waltham, MA (US); Swati GUPTA, Waltham, MA (US); Ryan LIM, Waltham, MA (US); Liwei LI, Waltham, MA (US)

(21) Appl. No.: 18/467,362

(22) Filed: Sep. 14, 2023

Related U.S. Application Data
(63) Continuation of application No. 16/862,214, filed on Apr. 29, 2020, now Pat. No. 11,819,042.
Application No. 16/862,214 is a continuation of application No. 16/251,993, filed on Jan. 18, 2019, now Pat. No. 10,743,567.
Application No. 16/251,993 is a division of application No. 15/524,015, filed on May 3, 2017, now Pat. No. 10,463,062.
(60) Provisional application No. 62/075,644, filed on Nov. 5, 2014.

(51) Int. Cl. A23L 2/60; A23L 27/30; C12N 9/02; C12N 9/10; C12P 19/56

(52) U.S. Cl. CPC A23L 2/60; A23L 27/36; C12N 9/0073; C12N 9/1048; C12N 9/1051; C12P 19/56; C12Y 114/13079; C12Y 204/01017; A23V 2002/00

(57) ABSTRACT

The invention provides methods for making steviol glycosides, including RebM and glycosylation products that are minor products in stevia leaves, and provides enzymes, encoding polynucleotides, and host cells for use in these methods. The invention provides engineered enzymes and engineered host cells for producing steviol glycosylation products, such as RebM, at high purity and/or yield. The invention further provides methods of making products containing steviol glycosides, such as RebM, including food products, beverages, oral care products, sweeteners, and flavoring products.

### RELATED APPLICATIONS

This application is a continuation of U.S. patent application Ser. No. 16/862,214, filed Apr. 29, 2020 and is now U.S. Pat. No. 11,819,042, which is a continuation of U.S. patent application Ser. No. 16/251,993, filed Jan. 18, 2019 and is now U.S. Pat. No. 10,743,567, which is a divisional of U.S. patent application Ser. No. 15/524,015, filed May 3, 2017 and is now U.S. Pat. No. 10,463,062, which is a National Stage of PCT/US15/59273, filed Nov. 5, 2015, which claims priority to and the benefit of U.S. Provisional Application No. 62/075,644, filed Nov. 5, 2014, each of which is hereby incorporated by reference in its entirety.

### TECHNICAL FIELD

The present disclosure relates to enzymes, including engineered enzymes, encoding polynucleotides, host cells, and methods for producing steviol glycosides.

### SEQUENCE LISTING

The application contains a sequence listing, which has been submitted in XML format via EFS-Web. The contents of the XML copy named “MAN-003D3_Sequence_Listing”, which was created on Sep. 9, 2023 and is 81,920 bytes in size, the contents of which are incorporated herein by reference in their entirety.

### BACKGROUND

High intensity sweeteners possess a sweetness level that is many times greater than the sweetness level of sucrose. They are essentially non-caloric and are commonly used in diet and reduced-calorie products, including foods and beverages. High intensity sweeteners do not elicit a glycemic response, making them suitable for use in products targeted to diabetics and others interested in controlling their intake of carbohydrates.

Steviol glycosides are a class of compounds found in the leaves of Stevia rebaudiana Bertoni, a perennial shrub of the Asteraceae (Compositae) family native to certain regions of South America. They are characterized structurally by a single base, steviol, differing by the presence of carbohydrate residues at positions C13 and C19. They accumulate in Stevia leaves, composing approximately 10% to 20% of the total dry weight. On a dry weight basis, the four major glycosides found in the leaves of Stevia typically include stevioside (9.1%), rebaudioside A (3.8%), rebaudioside C (0.6-1.0%) and dulcoside A (0.3%). Other known steviol glycosides include rebaudioside B, C, D, E, F and M, steviolbioside and rubusoside.

The minor glycosylation product rebaudioside M is estimated to be about 200-350 times more potent than sucrose, and is described as possessing a clean, sweet taste with a slightly bitter or licorice aftertaste. Prakash I, et al., Development of Next Generation Stevia Sweetener: Rebaudioside M, Foods 3(1), 162-175 (2014). RebM is of great interest to the global food industry.

Although methods are known for preparing steviol glycosides from Stevia rebaudiana, many of these methods are unsuitable for use commercially and/or are not sustainable. Accordingly, there remains a need for simple, efficient, and economical methods for preparing compositions comprising steviol glycosides, including highly purified steviol glycoside compositions. Further, methods are needed for producing substantial amounts of the minor glycosylation products, including products having a plurality of glycosylations, such as Reb A, Reb D, Reb E, Reb I, RebM, and others.

### SUMMARY OF THE INVENTION

In various aspects, the invention provides methods for making steviol glycosides, including Reb D and RebM and glycosylation products that are minor products in stevia leaves, and provides enzymes, encoding polynucleotides, and host cells for use in these methods. The invention provides engineered enzymes and engineered host cells for producing steviol glycosylation products, such as Reb D and RebM, at high purity and/or yield. The invention further provides methods of making products containing steviol glycosides, where the products include food products, beverages, oral care products, sweeteners, flavoring products, among others.

In various aspects and embodiments, the invention provides enzymes, encoding polynucleotides, host cells, and methods for producing steviol glycosides having a plurality of glycosylations at C13 and/or C19. The steviol glycosides may have 2, 3, 4, 5, 6, 7, 8 or more glycosylations. In various embodiments, the glycosylations are selected from: C13-0, C19-O, 1-2′ (at C-13 and/or C19 of steviol), and 1-3′ (at C13 and/or C19 of steviol). Exemplary enzymes to perform these glycosylations are listed in Table 8, and include enzymes that catalyze C13-O glycosylations of steviol (e.g., SrUGT85C2), C19-O glycosylations of steviol (e.g., SrUGT74G1), 1-2′ glycosylations of steviol glycosides (e.g., SrUGT91D1, SrUGT91D2, OsUGT1-2), and 1-3′ glycosylations of steviol glycosides (e.g., SrUGT76G1). Numerous derivatives that can be used in various embodiments are disclosed herein, including enzymes identified herein as MbUGTc13 (SEQ ID NO:51), MbUGTc19 (SEQ ID NO:8), MbUGTc19-2 (SEQ ID NO:46), MbUGT1-2 (SEQ ID NO:9), MbUGT1,2-2 (SEQ ID NO:45), and MbUGT1-3 (SEQ ID NO:10), and derivatives thereof. In some embodiments, the invention provides host cells that express at least 2, 3, or 4 UGT enzymes for performing these glycosylations in vivo on the steviol substrate. Various steviol glycoside products that can be produced according to embodiments of the invention are shown in FIG. 28, FIG. 29, FIG. 30, FIG. 31 and Table 10, and these include Reb M, Reb D, Reb E, and Reb I. In accordance with embodiments of the invention, these steviol glycosides can be produced at high yields in bacterial host cells, such as E. coli, including at temperatures suitable for E. coli growth and metabolism.

In some aspects, the invention provides modified UGT enzymes having an increase in 1-2′ glycosylating activity at C19 of Rebaudioside A (RebA) as compared to its parent UGT enzyme, and without substantial loss of 1-2′ glycosylating activity at C13 of steviolmonoside. Such enzymes can provide for increased carbon flux to RebD. Further, the invention provides modified UGT enzymes having an increase in 1-3′ glycosylating activity at C19 of Rebaudioside D (RebD) as compared to its parent UGT enzyme, without substantial loss of 1-3′ glycosylating activity at C13 of stevioside. Such enzymes can provide for increased carbon flux to RebM.

In accordance with the present disclosure, production of steviol glycosides is engineered in host cells through the production of various pathway modules from glycolysis to steviol, and further to steviol glycosides, and which can be optimized and balanced to promote carbon flux to steviol and then to Reb D or RebM (or other glycosylation product) as the main glycosylation product.

In another aspect, the invention provides a method for making RebD. The method comprises providing a host cell producing RebD from steviol through a plurality of uridine diphosphate dependent glycosyltransferase enzymes (UGT), and culturing the host cell under conditions for producing the RebD. The UGT enzymes comprise a modified UGT enzyme having an increase in 1-2′ glycosylating activity at C19 of Rebaudioside A (RebA) as compared to its parent UGT enzyme, without substantial loss of 1-2′ glycosylating activity at C13 of steviolmonoside. In certain embodiments, the 1-2′ glycosylating activity at C19 of Rebaudioside A (RebA) is equal to or better than the 1-2′ glycosylating activity at C13 of steviolmonoside.

In another aspect, the invention provides a method for making RebM. The method comprises providing a host cell producing RebM from steviol through a plurality of uridine diphosphate dependent glycosyltransferase enzymes (UGT), and culturing the host cell under conditions for producing the RebM. The UGT enzymes comprise one or more of: (a) a modified UGT enzyme having an increase in 1-2′ glycosylating activity at C19 of Rebaudioside A (RebA) as compared to its parent UGT enzyme, without substantial loss of 1-2′ glycosylating activity at C13 of steviolmonoside; and (b) a modified UGT enzyme having an increase in 1-3′ glycosylating activity at C19 of Rebaudioside D (RebD) as compared to its parent UGT enzyme, without substantial loss of 1-3′ glycosylating activity at C13 of stevioside. In certain embodiments, the 1-2′ glycosylating activity at C19 of Rebaudioside A (RebA) is equal to or better than the 1-2′ glycosylating activity at C13 of steviolmonoside. Alternatively or in addition, the 1-3′ glycosylating activity at C19 of Rebaudioside D (RebD) is equal to or better than the 1-3′ glycosylating activity at C13 of stevioside.

20) In some embodiments, the invention provides modified SrUGT76G1 enzymes, which provide for 1-3′ glycosylating activity of stevioside and RebD, including enzymes having an amino acid substitution at position 200 of the wild type enzyme (e.g., L200A or L200G), which exhibit substantial improvement in activity.

In other aspects and embodiments, the invention provides circular permutants of UGT enzymes (as well as encoding polynucleotides), which can provide novel substrate specificities, product profiles, and reaction kinetics over the wild-type enzymes. The circular permutants can be expressed in host cells for production of steviol glycosides as described herein. Thus, in various embodiments the microbial cell expresses at least one UGT enzyme that is a circular permutant of a wild-type or parent UGT enzyme. A circular permutant retains the same basic fold of the parent enzyme, but has a different position of the N-terminus (e.g., “cut-site”), with the original N- and C-termini connected, optionally by a linking sequence. For example, in the circular permutants, the N-terminal Methionine is positioned at a site in the protein other than the natural N-terminus. For example, the invention provides circular permutants of OsUGT1-2 and SrUGT74G1, which can be further modified as described herein for production of glycosylation products of steviol.

In another aspect, the invention provides a method for production of steviol glycosides having at least 4 glycosylations in E. coli. In accordance with the invention, the E. coli cell comprises a plurality of UGT enzymes, which may include one or more enzymes described herein, that together perform at least 4, at least 5, or at least 6, sequential glycosylation reactions. As disclosed herein, the glycosylation substrates and lower glycosylation products accumulate in the E. coli cell sufficiently to allow downstream reactions to proceed at an acceptable rate, with a high majority of the glycosylation products ultimately accumulating extracellularly. The steviol glycosides can be purified from media components. Thus, E. coli is a desirable host for production of steviol glycosides that require several glycosylation reactions of the steviol scaffold.

In still other aspects, the invention provides methods for production of steviol glycosides (including RebM, Reb D, Reb E, Reb I, and others) in E. coli. While many of the enzymes known for production of steviol in host cells are plant enzymes, which often have optimal temperatures in the range of 20-24° C., E. coli growth rate and metabolism are optimal at higher temperatures. The present disclosure enables production of steviol glycosides at high yield in E. coli, by enabling enzyme productivity at temperatures above 24° C., such as from 24° C. to 37° C., or from 27° C. to 37° C., or from 30° C. to 37° C. In various embodiments, the disclosure provides alternative or engineered GGPPS, KS, CPPS, KO, and KAH enzymes for production of steviol or steviol glycosides in E. coli or other microbial host.

Other aspects and embodiments of the invention will be apparent from the following detailed disclosure.

### DESCRIPTION OF THE FIGURES

FIG. 1 shows the chemical structure of Rebaudioside M (RebM), a minor component of the steviol glycoside family, and which is a derivative of the diterpenoid steviol (box) with six glucosyl-modification groups.

FIG. 2 shows pathway modules to RebM. Glycolysis and MEP pathways are treated as one module, and the downstream kaurene biosynthesis pathway is shown as the second module. Biosynthesis of steviol is shown as the third module. The fourth module is for the glycosylation of steviol and the RebM biosynthetic pathway. The fifth module to support enhanced UDP-glucose production is also shown.

FIG. 3 shows an exemplary pathway for steviol glycoside production, including to RebM. PsCKS is a bifunctional copalyl diphosphate and kaurene synthase (from Phaeosphaeria sp.) which acts on geranylgeranyl diphosphate (synthesized from IPP and DMAPP by Taxus canadensis GGPP synthase, not shown). SrKO is Stevia rebaudiana kaurene oxidase and AtKAH is an Arabidopsis thaliana P450 with steviol monooxygenase activity. Solid arrows are known UGT activities. Arrows with dotted line borders are predicted reactions based on demonstrated activities on other substrates in vitro. MbUGT1-2 is a novel UGT enzyme designed in this disclosure.

FIG. 4A, FIG. 4B, FIG. 4C, and FIG. 4D show kaurene production profiles from engineered E. coli cells. FIG. 4A and FIG. 4B are kaurene production from CPPS/KS enzymes selected from plant Stevia rebaudiana (SrCPPS and SrKS) and Physcomitrella patens (PpCK), respectively. FIG. 4C and FIG. 4D are strains constructed with enzymes selected from fungus species Gibberella fujikuroi (GfCK) and Phaeosphaeria sp. (PsCK), respectively.

FIG. 5 shows GC profiles from strains constructed from different KS enzymes. The pathway is shown in FIG. 2. The figure in the box (left inset) is the magnified chromatograph to show the byproduct accumulation. The GC profile and corresponding MS spectra show that the KS enzymes can be non-specific vis-à-vis product profile. Other terpenoid byproducts were produced with similar MS characteristics as kaurene. In all three pathways the major product is kaurene. The authenticity of kaurene is confirmed by comparison to MS spectra and NMR data reported in previously published literature. The MS spectra from all the byproduct show a characteristic 272 molecular ion.

FIG. 6A, FIG. 6B, FIG. 6C, and FIG. 6D show the product profile from engineered strains. Shown are the production profiles from different downstream pathway expression levels under different upstream pathway modulation. The byproducts are the same as those shown in FIG. 5. Genotype details of each strain are in Table 2.

FIG. 7 shows that a strain (Strain 47 in table 2, Ch1TrcMEP-Ch1T7PsCKG) with properly balanced modules enabling kaurene biosynthesis, is capable of multi-gram-per-liter scale productivity of kaurene in a 2L bioreactor.

FIG. 8 shows that indole accumulation is inversely correlated to kaurene production across engineered strains.

FIG. 9A, FIG. 9B, and FIG. 9C show redesign and characterization of SrKO enzyme. FIG. 9A is the N-terminal transmembrane region analysis and truncations with modifications to SrKO. FIG. 9B shows schematics of designed SrKO/SrCPR enzyme constructs. FIG. 9C shows protein expression from different engineered constructs in E. coli: (1) WT, (2) WT+(MA)KO—O—CPR, (3) WT+(MA)KO—O—CPR, (4) WT+(MA)KO-L-CPR, (5) WT+(MA)KO-L-CPR, (6) WT+(8RP)KO—O—CPR, (7) WT+(8RP)KO—O—CPR, (8) WT+(8RP)KO-L-CPR, (9) WT+(8RP)KO-L-CPR.

FIG. 10 shows the kaurenoic acid productivity of SrKO in linker or operon configuration with SrCPR in strain 47 background.

FIG. 11A and FIG. 11B illustrates the MMME landscape exploration of SrKO constructs under varying plasmid copy numbers and promoter strength. Imbalanced modules show less or no kaurenoic acid accumulation, with an associated increase in upstream kaurene accumulation instead.

FIG. 12 shows design of a CYP450 expression module to screen for optimum enzyme variants, N-terminal truncations, and point mutations of KO, KAH, or CPR genes. The two P450s and the CPR enzyme are expressed in a polycistronic operon under various promoter strengths in either plasmid or chromosomally-integrated format.

FIG. 13A and FIG. 13B show point mutants of AtKAH enzyme, as represented by fold-change in kaurenoic acid hydroxylase activity relative to wild-type AtKAH.

FIG. 14 shows a series of engineered AtKAH that demonstrate improved steviol productivity and eventual complete conversion of kaurenoic acid to steviol.

FIG. 15 shows that, in a properly balanced module, the two P450s (AtKAH and SrKO) and the co-factor CYP450 reductase (SrCPR) are capable of complete conversion of kaurene through kaurenoic acid through to steviol.

FIG. 16 demonstrates increased UDP-glucose production in E. coli using a model system: glycosylation of a small molecule caffeic acid with terpene producing E. coli strains engineered for increased UDP-glucose production, producing caffeic acid 3-glucoside using Vitis vinifera glycosyltransferase 2 (VvGT2) overexpressed from a pET plasmid. The improvement in glycosylated caffeic acid titers compared to the unmodified background strain shows an increase in the UDP-glucose substrate pool to support glycosylation. Strain 1 is Strain 47 (Table 2) with knock-outs of the galactose catabolic module (galETKM), UDP-sugar pyrophosphatase (ushA), phosphoglucomutase (pgm), glycose-1 phosphatase (agp), β-galactosidase (lacZ), and overexpressing sucrose phosphorylase (spl) under the Trc promoter (see Table 7). Strain 2 is Strain 47 (Table 2) with knock-outs of the galactose catabolic module (galETKM), UDP-sugar pyrophosphatase (ushA), phosphoglucomutase (pgm), glycose-1 phosphatase (agp), β-galactosidase (lacZ), and overexpressing and sucrose phosphory lase (spl) under the T7 promoter (see Table 7).

FIG. 17 shows the process for identification of an optimum glycosylation module incorporating all four UGT activities. All 24 possible combinations are rapidly assembled in three different plasmids, enabling expression at three different levels, for a total of 72 potential constructs.

FIG. 18A and FIG. 18B show in vivo production of RebM. FIG. 18A shows product titers of steviol glycoside from E. coli culture. FIG. 18B shows LC/MS trace showing RebM identification. Negative control strain has been modified to produce steviol and increased UDP-glucose pools, while 4UGT strain is the negative control strain plus four UGTs.

FIG. 19 shows a homology model of OsUGT1-2 (1-2′ glycosylating enzyme from rice, Oryza sativa), as a starting point for circular permutant design.

FIG. 20A, FIG. 20B, and FIG. 20C show linkers for UGT circular permutants, to connect the natural N and C-termini. Three different linkers are shown: FIG. 20A shows YKDDSGYSSSYAAAAGM (SEQ ID NO:48) attaching the existing sequence, FIG. 20B shows YKDAAGM (SEQ ID NO:49), creating an intermediate-length loop, and FIG. 20C shows YGSGM (SEQ ID NO:50), creating a minimal loop.

FIG. 21 illustrates criteria for selection of new N- and C-termini for the UGT circular permutant. Positions for new termini should be: (1) solvent exposed and away from the active site to minimize perturbation, (2) close to the middle of the sequence to maximize difference with the parental sequence, and (3) have amino acids often found at existing circular permutant division points (Lo, et al., 2012, PLOS One 7(2):e31791). New N-termini at G198, K240, G250, and G259 fit these criteria.

FIG. 22 shows 1-2′ glycosylating activity for the first round of circular permutants of OsUGT1-2. The numbers indicate the location of the cut site in the parental sequence used to generate novel positions for N- and C-termini, while the L/M/S designation describes the long/medium/short linkers (which are described in FIG. 20A, FIG. 20B, and FIG. 20C).

FIG. 23A and FIG. 23B show refinement of the 1-2′ UGT circular permutant (MbUGT1-2). Modifications to the cut site and linker length demonstrate significantly enhanced activity on at least one of the substrates possible for this enzyme. Number before L (eg. xxxL) indicates new cut site position, while number after L (eg. 1Lxx) indicates a new linker length in background with the 198 cut site [BL21=negative no UGT control].

FIG. 24 shows point mutations in the MbUGT1-2 enzyme. Point mutations show increased activity on substrate steviolmonoside, demonstrating the potential for improving UGT enzymes generated by circular permutization [BL21=negative no UGT control].

FIG. 25 shows point mutations in the MbUGT1-2 enzyme. Point mutations show increased activity on substrate rebaudioside A, demonstrating the potential for improving UGT enzymes generated by circular permutization. [BL21=negative no UGT control].

FIG. 26 shows that point mutations that are beneficial to the MbUGT1-2 enzyme do not, when translated to the appropriate amino acid residue in the parental UGT enzyme, result in neutral or even deleterious effects on activity. This demonstrates that circular permutants have the potential for unique improvements and evolution compared to the parent enzyme, brought about by shuffling of the sequence into a novel arrangement not previously selected for by natural selection. [BL21=negative no UGT control].

FIG. 27 shows a chimeric UGT with C13-O-glycosylating activity, created by fusing the N-terminus of SrUGT85C2 and the C-terminus of SrUGT76G1.

FIG. 28 is a summary of possible reactions (marked by arrows) catalyzed by SrUGT85C2 (i.e., C13-O-glycosylations).

FIG. 29 is a summary of possible reactions (marked by arrows) catalyzed by SrUGT74G1 (i.e., C19-O-glycosylations).

FIG. 30 is a summary of possible reactions (marked by arrows) catalyzed by MbUGT1-2 (i.e., 1-2-glycosylations).

FIG. 31 is a summary of possible reactions (marked by arrows) catalyzed by SrUGT76G1 (i.e., 1-3-glycosylations).

FIG. 32A, FIG. 32B, and FIG. 32C show point mutations in SrUGT85C2 enzyme versus altered activity on steviol substrate. [BL21=negative no UGT control].

FIG. 33A, FIG. 33B, and FIG. 33C show point mutations in SrUGT85C2 enzyme versus altered activity on C19-glucopyranosyl steviol substrate. [BL21=negative no UGT control].

FIG. 34A and FIG. 34B show C19-O-glycosylating activity for the first round of circular permutants of SrUGT74G1. The numbers indicate the location of the cut site in the parental sequence used to generate novel N- and C-termini, while the wt/L/S designation describes wild-type/long/short linkers (where ‘wt’ indicates a simple fusion of existing N- and C-termini sequences with no alteration).

FIG. 35 shows point mutations in SrUGT76G1 enzyme versus altered activity on stevioside substrate. [BL21=negative no UGT control].

FIG. 36 shows point mutations in SrUGT76G1 enzyme versus altered activity on rebaudioside D substrate. [BL21=negative no UGT control].

FIG. 37A and FIG. 37B show 1-3′ glycosylating activity for the first round of circular permutants of SrUGT76G1. The numbers indicate the location of the cut site in the parental sequence used to generate novel N- and C-termini, while the L/S designation describes long/short linkers.

FIG. 38A and FIG. 38B show UGT alignment and secondary structure, anchored to 2VCE which is an Arabidopsis UGT with a solved crystal structure. Q0DPB7-ORYSJ is OsUGT1-2. Boxed is the position of the 76G1-L200A point mutation, which promotes significantly improved activity. Also shown in boxes is the conserved PSPG motif.

FIG. 39 shows alternate GGPPS enzymes tested in vivo for performance at 22° C., 30° C., and 37° C.

FIG. 40 shows alternate CPPS/KS pairs tested in vivo for performance at 22° C., 30° C., and 37° C.

FIG. 41 shows the titer of Reb M in comparison to steviol and other glycosylation products, using a selected strain at 22° C.

FIG. 42 shows that the majority of Reb M accumulates extracellularly. Left Panel shows the titer of Reb M and steviol glycosides inside and outside of the cell. Right Panel shows the same data as the percent of each compound observed extracellularly.

FIG. 43A, FIG. 43B, and FIG. 43C show screening of UGT85C2 mutants at 22, 30, and 34° C., based on production of steviolmonoside. FIG. 43A and FIG. 43B show the panel of mutants at 34° C., and FIG. 43C shows select mutations screened for steviolmonoside production at 22, 30, and 34° C.

FIG. 44A and FIG. 44B show screening of 74G1 circular permutants for activity at 30 and 34° C. FIG. 44A shows activity on Steviol, and FIG. 44B shows activity on steviolbioside.

FIG. 45 shows screening of AtKAH point mutants for activity at 22, 26, and 30° C.

FIG. 46A and FIG. 46B show in vitro screening of MbUGT1-2 recombination mutants at 30 and 34° C. FIG. 46A shows conversion of Reb A to Reb D. FIG. 46B shows conversion of Steviolmonoside to 13C Steviolbioside.

FIG. 47 shows kaurene production at 30° C. across various module constructs.

FIG. 48 shows kaurene production at 34° C. across various module constructs.

FIG. 49 shows production of Steviol at 30° C. across a library of AtKAH point mutations.

FIG. 50 shows production of Steviol at 34° C. across a library of AtKAH point mutations.

FIG. 51A and FIG. 51B show activities of MbUGT1-2 circular permutants at 30° C., 34° C., and 37° C. FIG. 51A shows conversion of Reb A to Reb D. FIG. 51B shows conversion of Steviolmonoside to 13C Steviolbioside.

FIG. 52 shows activities of UGT85C2 mutants for conversion of Steviol to 13C Steviolmonoside at 30° C., 34° C., and 37° C.

FIG. 53 shows activities of UGT76G1 mutants for conversion of Reb D to 13C Reb M at 30° C., 34° C., and 37° C.

FIG. 54 shows activities of UGT74G1 circular permutants for conversion of Steviolbioside to 13C Stevioside at 30° C., 34° C., and 37° C.

### DETAILED DESCRIPTION OF THE INVENTION

In various aspects, the invention provides methods for making steviol glycosides, including RebM, RebD, and glycosylation products that are minor products in stevia leaves, and provides enzymes, encoding polynucleotides, and host cells for use in these methods. The invention provides engineered enzymes and engineered host cells for producing steviol glycosylation products at high purity and/or yield. The invention further provides methods of making products containing steviol glycosides, such as RebM or RebD, including food products, beverages, oral care products, sweeteners, flavoring products, among others. Such steviol glycoside-containing products can be made at reduced cost by virtue of this disclosure.

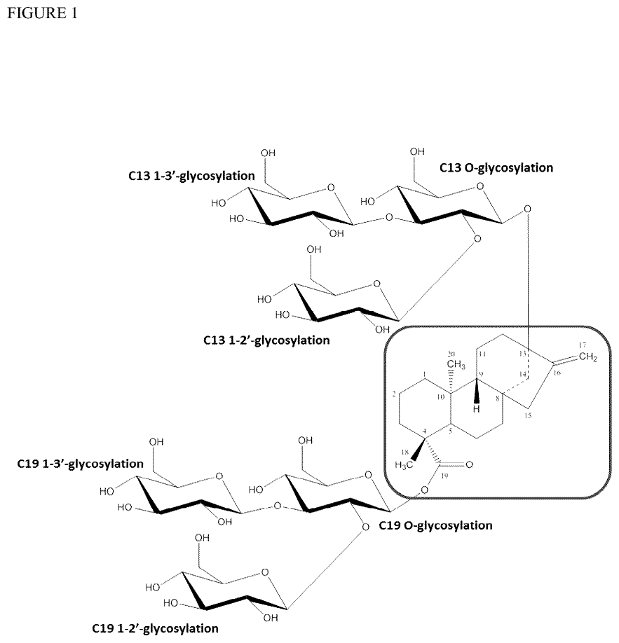

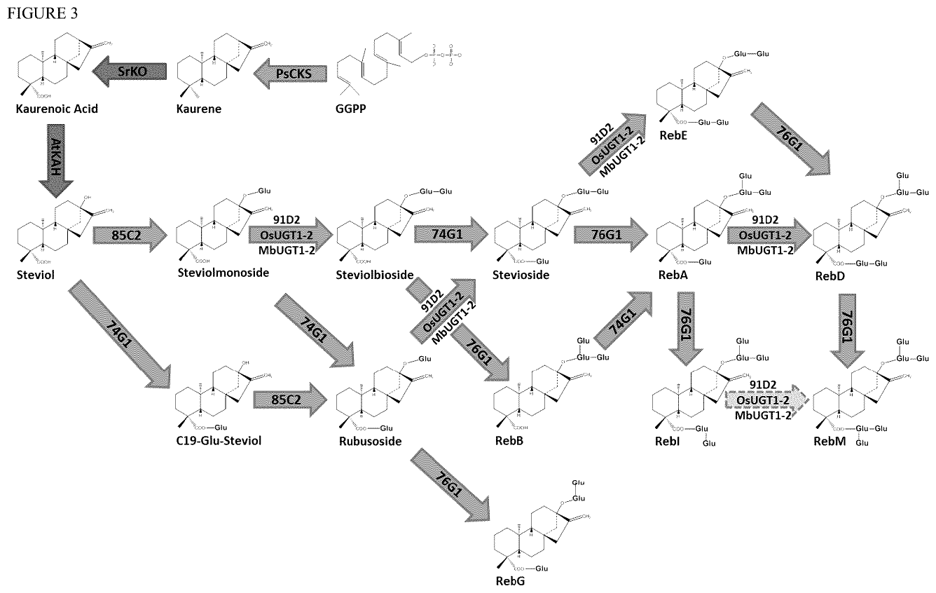

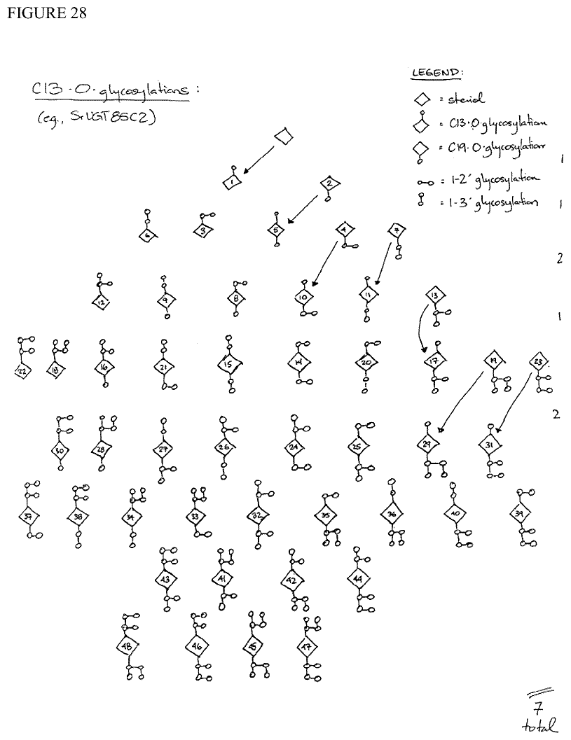

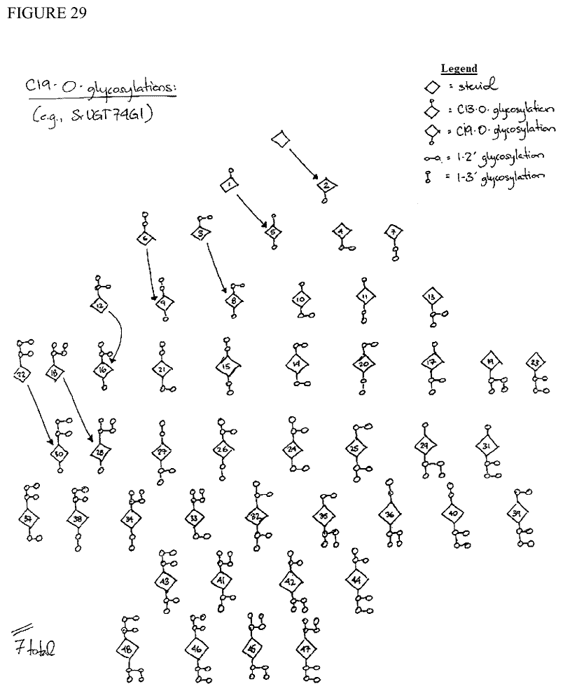

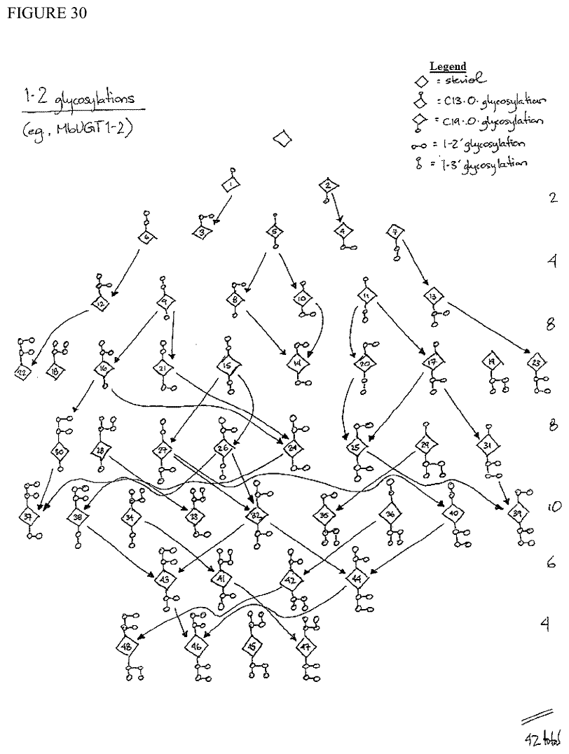

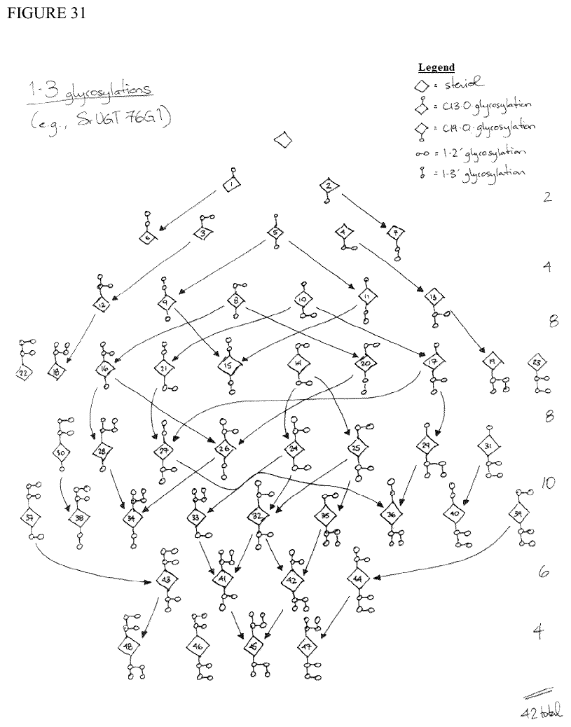

RebM is illustrated in FIG. 1, with the steviol scaffold (a diterpenoid) shown boxed. RebM contains six glycosylations: (1) a C13 O-glycosylation, (2) a C13 1-2′ glycosylation, (3) a C13 1-3′ glycosylation, (4) a C19 O-glycosylation, (5) a C19 1-2′ glycosylation, and (6) a C19 1-3′ glycosylation. Pathways from geranylgeranyl pyrophosphate (GGPP) to RebM are illustrated in FIG. 3. GGPP produced from IPP and DMAPP (products of the MEP or MVA pathways), is converted to kaurene by the action of copalyl synthase and kaurene synthase, which can be present as a bifunctional enzyme in some embodiments. Steviol is produced from kaurene by the action of two P450 enzymes, kaurene oxidase and kaurenoic acid hydroxylase, which are regenerated by one or more P450 reductase enzymes. After production of steviol, a series of glycosylation reactions at C13 and C19 are capable of producing various steviol glycoside products, including the hexaglycosylated RebM. Various other glycosylation products are possible (as shown in FIG. 3), and as illustrated in FIG. 28, FIG. 29, FIG. 30, and FIG. 31 known UGT glycosylation enzymes are each capable of acting on a number of substrates. Thus the fidelity, relative reaction rates, expression levels, and availability of substrate will affect the relative yields of the glycosylation products. For example, both UGT91D2 and OsUGT1-2 are 1-2′ glycosylating enzymes that can produce steviolbioside from steviolmonoside (by action at C13), as well as RebD from RebA (by action at C19). Further, UGT76G1 is a 1-3′ glycosylating enzyme that can produce RebA from stevioside (by action at C13), as well as RebM from RebD (by action at C19). Tables 8, 9, and 10 show the various possible steviol glycosides that may result from the six glycosylation reactions, as well as enzymes for each reaction. Table 1 lists various enzymes that may be used for the production of steviol glycosides. Amino acid sequences are also provided herewith, each of which can optionally include an alanine inserted or substituted at position 2 to decrease turnover in the cell. Certain GGPPS sequences further contain two additional residues (VD) at the end of the sequence, which are not believed to have any deleterious effect, and may be omitted in certain embodiments.

Thus, in some aspects, the invention provides enzymes, encoding polynucleotides, and host cells engineered for maximizing the production of the desired steviol glycoside (e.g., RebM). For example, this disclosure provides modified UGT enzymes having an increase in 1-2′ glycosylating activity at C19 of Rebaudioside A (RebA) as compared to the parent UGT enzyme, and without substantial loss of 1-2′ glycosylating activity at C13 of steviolmonoside. Such enzymes may provide for increased carbon flux to RebD. Further, this disclosure provides modified UGT enzymes having an increase in 1-3′ glycosylating activity at C19 of Rebaudioside D (RebD) as compared to the parent UGT enzyme, without substantial loss of 1-3′ glycosylating activity at C13 of stevioside. Such enzymes may provide for increased carbon flux to RebM. In some aspects and embodiments, and without wishing to be bound by theory, the invention provides for modified UGT enzymes with substrate binding pockets that are better able to accommodate substrates (including larger substrates), thereby increasing the rate of activity (e.g., rate of substrate binding and turnover) with more highly glycosylated steviol substrates such as RebA or RebD.

The invention in some aspects provides for a controlled glycosylation pathway that produces largely RebM as a glycosylation product. For example, in some embodiments, the invention provides a method for making RebM in microbial cells, where the RebM: RebD ratio is greater than about 1:1, or greater than about 1:0.5, or greater than about 1:0.25, or greater than about 10:1, or greater than about 25:1, or greater than about 50:1. In some embodiments, RebD is produced at less than about 20%, or at less than about 10%, or at less than about 5%, or at less than about 1% of the RebM yield, or is not detectable in the isolated steviol glycosylation products. Because RebD can be difficult to separate from RebM, or can add significant purification costs if such separation is necessary, products with low levels of RebD are desirable in some embodiments. In some embodiments, RebM represents at least about 25% by weight of the steviol glycosylation products produced by the cell, or at least about 50% by weight of the glycosylation products, or at least about 75% by weight of the glycosylation products, or at least about 80% by weight of the glycosylation products, or at least about 85% by weight of the glycosylation products, or at least about 90% by weight of the glycosylation products, or at least about 95% by weight of the steviol glycosylation products.

The glycosylation pathways involve a 13-O glycosylation, a 19-O glycosylation, as well as one or more 1-2′ glycosylations and/or one or more 1-3′ glycosylations at C13 and/or C19 of steviol. The term “steviol glycoside(s)” refers to a glycoside of steviol, including, but not limited to, steviolmonoside, steviolbioside, rubusoside, dulcoside B, dulcoside A, rebaudioside B, rebaudioside G, stevioside, rebaudioside C, rebaudioside F, rebaudioside A, rebaudioside E, rebaudioside H, rebaudioside L, rebaudioside K, rebaudioside J, rebaudioside M, rebaudioside D, rebaudioside N, rebaudioside O. The chemical identities of these steviol glycosides are known, and are described for example, in Table 10, as well as in WO 2014/122227, which is hereby incorporated by reference in its entirety.

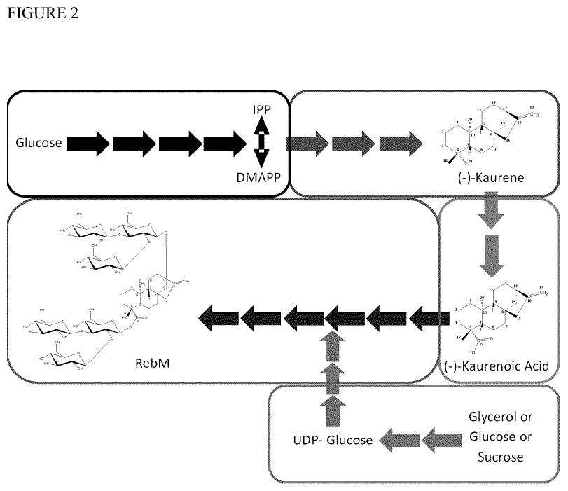

In accordance with the present disclosure, production of steviol glycosides is engineered in host cells through the production of various pathway “modules,” as illustrated in FIG. 2, and which can be optimized and balanced to promote carbon flux to steviol and then a desired glycosylation product (such as RebM or RebD) as the main glycosylation product. By grouping enzymes with similar turnovers into a subset, or module, and equalizing the turnover of the different subsets by adjusting concentrations/activities of enzymes, the ratio of pathway turnover to resource expenditure can be optimized.

The first pathway module comprises enzymes in the MEP or MVA pathways, which produce IPP and DMAPP. The MEP and MVA pathways may be endogenous to the organism. and these pathways may be increased and balanced with downstream pathways by providing duplicate copies of certain rate-limiting enzymes. IPP and DMAPP act as a substrate for the production of (−)-kaurene (e.g., by separate copalyl synthase and kaurene synthase enzymes, or a bifunctional enzyme), which is the second pathway module. A third pathway module converts (−)-kaurenoic acid to steviol by the action of two P450 enzymes (e.g., kaurene oxidase (KO) and kaurenoic acid hydroxylase (KAH)) and one or more P450 reductase enzymes. Exemplary enzymes that catalyze production of GGPP and its conversion through to steviol are listed in Table 1. Steviol is then glycosylated to the final product by a UDP enzyme module. An additional module includes genes that enhance production of the UDP-glucose substrate. In various embodiments of the invention, these modules are each present as mono- or poly-cistronic operons, which are each harbored on plasmids or are chromosomally integrated. In certain embodiments, the modules are configured for increased production of the desired end-product.

In one aspect, the invention provides a method for making a steviol glycoside, which is optionally RebM or RebD. The method comprises providing a host cell producing the steviol glycoside from steviol through a plurality of uridine diphosphate dependent glycosyltransferase enzymes (UGT), and culturing the host cell under conditions for producing the steviol glycoside. The UGT enzymes comprise one or more of: (a) a modified UGT enzyme having an increase in 1-2′ glycosylating activity at C19 of Rebaudioside A (RebA) as compared to its parent UGT enzyme, without substantial loss of 1-2′ glycosylating activity at C13 of steviolmonoside (e.g., when evaluated at 22° C., 27° C., or 30° C.); and (b) a modified UGT enzyme having an increase in 1-3′ glycosylating activity at C19 of Rebaudioside D (RebD) as compared to its parent UGT enzyme, without substantial loss of 1-3′ glycosylating activity at C13 of stevioside (e.g., when evaluated at 22° C., 27° C., or 30° C.).

In certain embodiments, the 1-2′ glycosylating activity at C19 of Rebaudioside A (RebA) is equal to or better than the 1-2′ glycosylating activity at C13 of steviolmonoside. Alternatively or in addition, the 1-3′ glycosylating activity at C19 of Rebaudioside D (RebD) is equal to or better than the 1-3′ glycosylating activity at C13 of stevioside.

In some embodiments, the modified UGT enzyme having 1-2′ glycosylating activity and/or the UGT enzyme having 1-3′ glycosylating activity does not exhibit a substantial loss of activity at C13, as compared to the parent enzyme. For example, the modified enzyme retains at least 50% of its activity at C13, or at least about 75% of its activity at C13, or at least about 80%, at least about 90%, or at least about 95% of its activity at C13 as compared to the parent (e.g., wild-type) enzyme (e.g., when evaluated at 22° C., 27° C., or 30° C.). In some embodiments, the enzyme has improved activity at C13, such as at least 2-fold or at least 3-fold improved activity at C13. The loss of, or improvement in, a glycosylation activity can be determined in vitro, for example in cell extracts with the substrate of interest added, or other in vitro or in vivo assay. For example, relative reaction rates may be determined in a strain that produces the steviol or steviol glycoside substrate(s) of interest. Exemplary assays for quantifying glycosylation activity are disclosed herein as well as in WO 2014/122227, which is hereby incorporated by reference.

While in some embodiments, the 1-2′ and 1-3′ glycosylation activities at C13 are sufficiently functional with the enzyme that performs these reactions at C19 (e.g., without any additional enzyme to perform these enzymatic steps), in other embodiments, the cell further expresses an enzyme to perform 1-2′ and/or 1-3′ glycosylation at C13. In some embodiments, a second enzyme is engineered to perform the 1-2′ and/or 1-3′ reactions at C13, even with loss of activity at C19.

In some embodiments, the cell expresses only one UGT enzyme having 1-2′ glycosylating activity at C13 of steviolmonoside, and/or expresses only one UGT enzyme having 1-3′ glycosylating activity at C13 of stevioside. In such embodiments, the enzyme can be engineered to enhance the reaction at C19, thereby pulling product toward C19 glycosylation products such as RebM, without the need for expression of additional enzymes that place a further metabolic burden on the cell.

In aspects and embodiments, the invention provides circular permutants of UGT enzymes (as well as encoding polynucleotides and methods of making circular permutants of UGT enzymes), which can provide novel substrate specificities, product profiles, and reaction kinetics over the parent (e.g., wild-type) enzymes. Without wishing to be bound by theory, circular permutants provide the opportunity to make the UGT binding pocket more open or accessible for larger substrates, such as steviol substrates having one or more glycosyl groups. In this manner, the invention allows for the glycosylation reactions on more glycosylated forms of steviol to proceed at rates similar to or even greater than reactions on less glycosylated (and thus smaller) substrates. The circular permutants can be expressed in host cells for production of steviol glycosides as described herein. Thus, in various embodiments the microbial cell producing the steviol glycoside (e.g., RebM or RebD) expresses at least one UGT enzyme that is a circular permutant of a parent (e.g., wild-type) UGT enzyme.

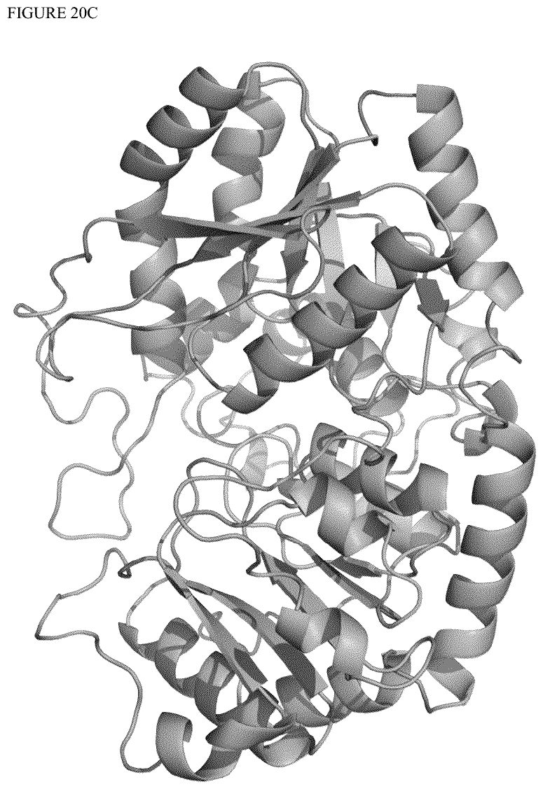

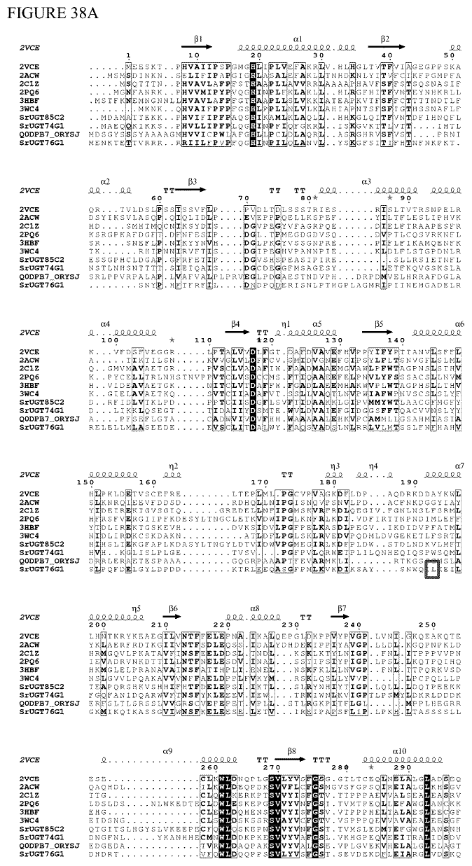

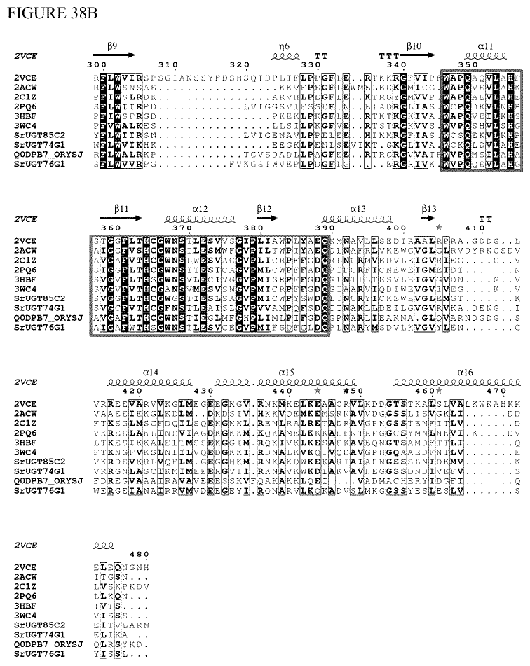

A circular permutant retains the same basic fold of the parent enzyme, but has a different position of the natural N-terminus (e.g., “cut-site”), with the original N- and C-termini connected, optionally by a linking sequence. An exemplary structure of a UGT enzyme (e.g., based on OsUGT1-2) is shown in FIG. 20A, FIG. 20B, and FIG. 20C. A UGT alignment and secondary structure elements are shown in FIG. 38A and FIG. 38B. For each circular permutant. the cut-site can be described with reference to the corresponding position of the parent sequence (e.g., wild-type sequence), by alignment of the permutant's N-terminal amino acids (e.g., N-terminal 50) or 100 amino acids) with the parent or wild-type sequence. As used herein. the “cut site” of a given circular permutant refers to the original position of the amino acid that is positioned at position 2 in the circular permutant (e.g., after the initiating Met), or position 3 of the circular permutant when an Alanine is inserted at position 2 to decrease protein turnover. Alignments for comparing global UGT sequences should be anchored around the conserved PSPG motif shown in FIG. 38A and FIG. 38B. The PSPG (plant secondary product glycosyltransferase) motif is a conserved region within plant UGTs that plays a role in binding the nucleotide-diphosphate-sugar donor molecule. Gachon et al., Plant secondary metabolism glycosyltransferases: the emerging functional analysis. Trends Plant Sci. 10:542-549 (2005). The most conserved residues in this motif in the UDPGT family show the pattern: WXPQXXXLXHXXXXAFXXHXGXXXXXEXXXXGXPXXXXPXFXXQ, of which the underlined histidine makes a critical contact to the diphosphate region. Finn R D, et al. Pfam: the protein families database. Nucleic Acids Res. 42:D222-230 (2014). Further, alignment around this motif is useful for describing point mutations that translate to beneficial properties for the UGT proteins as a class. For example, anchoring alignments to the tryptophan at the beginning of the motif, or the important histidine in the middle, may be used to describe point mutations relative to this sequence, which will be universal in plant GT1 UDP-glucose glycosyltransferases.

In some embodiments. the circular permutant is a circular permutant of UGT85C2 from Stevia rebaudiana. SrUGT85C2 is provided herein as SEQ ID NO:1. In some embodiments. the circular permutant is a circular permutant of OsUGT1-2 (SEQ ID NO:7). In some embodiments. the circular permutant is of UGT91D2 of Stevia rebaudiana (SEQ ID NO:5). In some embodiments. the circular permutant is of UGT74G1 of Stevia rebaudiana (SEQ ID NO:2). In some embodiments, the circular permutant is of UGT76G1 of Stevia rebaudiana (SEQ ID NO:3). In this manner, by changing the position of the N-terminus of the UGT enzyme. enzymes with novel substrate specificities and activity profiles can be created. For example, in some embodiments. the cut site is between amino acids 150 to 300, or in some embodiments between amino acids 190 and 260, or in some embodiments between residues 190 and 210. when the N-terminus of the circular permutant (e.g., N-terminal 50) amino acids) is aligned with the parent or wild-type enzyme. In other embodiments. the circular permutant has a cut site between amino acids 245 and 280 (e.g., position 272), or between amino acids 260 to 275. when the N-terminal 50 amino acids of the circular permutant are aligned with the parent or wild-type enzyme. In some embodiments. the new N-terminus is placed between local secondary structure elements (such as α-helices or β-sheets), and/or is placed at a loop structure of the wild-type enzyme. When selecting the desired position of the N-terminus, a Met is added to the cut-site as the initiating amino acid, and an Ala is optionally placed at the second position to decrease cellular turnover. The natural N and C-termini are linked, optionally with a linking sequence. Generally. the linking sequence is selected to provide flexibility (e.g., no defined secondary structure other than a potential loop). for example using a sequence consisting predominately or essentially of Gly. Ser, and/or Ala. In some embodiments. the linking amino acid sequence is from about 2 to about 25 amino acids in length, which may form a loop. The circular permutant may further comprise from 1 to about 30, or from about 1 to about 20, or from 1 to about 10, or from 1 to about 5 amino acid substitutions, deletions, or insertions with respect to the corresponding position of the parent or wild-type enzyme (e.g., based on the highest score local alignment). In some embodiments. the natural N-terminal Met is maintained at its new position in the molecule, or in other embodiments is deleted.

In some embodiments, at least one UGT enzyme is a chimeric UGT enzyme, in which the N-terminal domain of one UGT is combined with the C-terminal domain of a different UGT enzyme. For example the N-terminal and C-terminal domains are of two different enzymes selected from Table 9, and each domain may further comprise from one to ten amino acid substitutions, deletions, and/or insertions relative to the parent domain sequence. UGTs have two domains, a more variable N-terminal substrate binding (sugar acceptor) domain and a more conserved C-terminal UDP-glucose binding (sugar donor) domain. The N-terminal domain is mostly determinant of substrate specificity for the enzyme, but some specificity is controlled by the C-terminal domain. Each of these domains makes up roughly half of the protein.

In some embodiments. the UGT enzyme having 1-2′ glycosylating activity is OsUGT1-2 (SEQ ID NO:7). SrUGT91D2 (SEQ ID NO:5). SrUGT91D1 (SEQ ID NO:4). SrUGT91D2e (SEQ ID NO:6) (see Table 9) or derivative thereof. In some embodiments. the derivative has increased glycosylating activity at C19 of RebA. The UGT enzyme may generally have a level of identity that is greater than about 50%, greater than about 60%, greater than about 70%, greater than about 80%, greater than about 90%, or greater than about 95%, or greater than about 96, 97, 98, or 99% to one or more of OsUGT1-2, SrUGT91D2, SrUGT91D1, and SrUGT91D2e.

The similarity or identity of nucleotide and amino acid sequences, i.e. the percentage of sequence identity, can be determined via sequence alignments. Such alignments can be carried out with several art-known algorithms, such as with the mathematical algorithm of Karlin and Altschul (Karlin & Altschul (1993) Proc. Natl. Acad. Sci. USA 90: 5873-5877), with hmmalign (HMMER package, http://hmmer.wustl.edu/) or with the CLUSTAL algorithm (Thompson, J. D., Higgins, D. G. & Gibson, T. J. (1994) Nucleic Acids Res. 22, 4673-80). The grade of sequence identity (sequence matching) may be calculated using e.g. BLAST, BLAT or BlastZ (or BlastX). A similar algorithm is incorporated into the BLASTN and BLASTP programs of Altschul et al (1990) J. Mol. Biol. 215: 403-410. BLAST polynucleotide searches can be performed with the BLASTN program, score=100, word length=12.

BLAST protein searches may be performed with the BLASTP program, score=50, word length=3. To obtain gapped alignments for comparative purposes. Gapped BLAST is utilized as described in Altschul et al (1997) Nucleic Acids Res. 25: 3389-3402. When utilizing BLAST and Gapped BLAST programs. the default parameters of the respective programs are used. Sequence matching analysis may be supplemented by established homology mapping techniques like Shuffle-LAGAN (Brudno M., Bioinformatics 2003b, 19 Suppl 1:154-162) or Markov random fields.

In some embodiments the UGT enzyme having 1-2′ glycosylating activity is OsUGT1-2 or derivative thereof, and which is optionally a circular permutant of OsUGT1-2 comprising one or more amino acid substitutions, deletions, and/or insertions that increase 1-2′ glycosylating activity at C19 of RebA. For example, the 1-2′ glycosylating enzyme may have a cut site that aligns with or corresponds to a position within amino acids 190 to 210 of OsUGT1-2 (SEQ ID NO:7), and may be a position within amino acids 194 to 200 of SEQ ID NO:7 in some embodiments, such as position 195, 196, 197, or 199. The circular permutant may optionally have a linker sequence between the amino acids that correspond to the N-terminal and C-terminal residues of OsUGT1-2. The linker may vary in length, such as in the range of 2 to about 25 amino acids. For example, the linker may be from about 8 to about 20 amino acids in length, such as about 17 amino acids in some embodiments. In some embodiments. the circular permutant does not contain any linking sequence. The circular permutant may further contain from 1 to 20, or from 1 to 10, or from 1 to 5 amino acid substitutions, additions, or deletions from the wild-type sequence (determined by local alignment of the mutated sequence to OsUGT1-2). In some embodiments, an Ala is inserted or substituted at position 2 to decrease enzyme turnover in the cell. In some embodiments. the mutations collectively increase 1-2′ glycosylating activity at C19 of RebA (e.g., when evaluated at 22° C. 27° C. or 30° C.).

In some embodiments. the UGT enzyme having 1-2′ glycosylating activity is a circular permutant of OsUGT1-2, with a cut-site corresponding to position 195, 196, 197, 198, or 199 of OsUGT1-2. An exemplary circular permutant. named MbUGT1-2, is disclosed herein. The circular permutant may have amino acid substitutions at one or more of positions corresponding to positions 14, 16, 89, 185, 365, 366, 395, 396, 417, 420, 421, 422, 424, 427, 428, 430, 431, 432, 434 and/or 463 of the wild-type enzyme. In some embodiments. the circular permutant has an amino acid substitution at position 14, and such substitution may be an aromatic amino acid, such as Trp or Tyr. In these or other embodiments. the circular permutant has an amino acid substitution at position 366, and the substituted amino acid is optionally Pro. In these or other embodiments. the circular permutant has an amino acid substitution at position 420, and the substituted amino acid is optionally Glu. In these or other embodiments. the circular permutant has an amino acid substitution at position 421, and the substituted amino acid is optionally Phe. In these or other embodiments. the circular permutant has an amino acid substitution at position 424, and the substituted amino acid is optionally Asp. In these or other embodiments. the circular permutant has an amino acid substitution at position 427, and the substituted amino acid is optionally Glu. In these or other embodiments, the circular permutant has an amino acid substitution at position 428, and the substituted amino acid is optionally Glu. In these or other embodiments. the circular permutant has an amino acid substitution at position 432, and the substituted amino acid is optionally Tyr. His. Trp. Asp, or Glu. In some embodiments. the enzyme contains an insertion of from 2-5 amino acids between amino acids 424 and 427, such as the sequence Gly-Pro-Ser. In some embodiments. the UGT having 1-2′ glycosylating activity comprises the amino acid sequence of SEQ ID NO:9 (MbUGT1-2), or an enzyme having at least about 50% identity, at least about 60% identity, at least about 70% identity, at least about 80% identity, at least about 85% identity, or at least about 90% identity. or at least about 95% identity, or at least 96%, 97%. 98% or 99% identity to SEQ ID NO:9, and having 1-2′ glycosylating activity at one or more of C19 of RebA or C13 of steviolmonoside.

In some embodiments, the UGT enzyme having 1-2′ glycosylating activity is a circular permutant of OsUGT1-2, with a cut site corresponding to position 196 of OsUGT1-2. An exemplary circular permutant, named MbUGT1,2-2 (SEQ ID NO:45), is disclosed herein. The circular permutant has amino acid substitutions at one or more of positions 16, 422, 430, and 434 of the wild-type enzyme. In some embodiments, the circular permutant has an amino acid substitution at position 16, and such substitution may be an aromatic amino acid, such as Trp. In these or other embodiments, the circular permutant has an amino acid substitution at position 422, and the substituted amino acid is optionally Glu. In these or other embodiments, the circular permutant has an amino acid substitution at position 430, and the substituted amino acid is optionally Glu. In these or other embodiments, the circular permutant has an amino acid substitution at position 434, and the substituted amino acid is optionally His. In some embodiments, the enzyme does not contain any linking sequence between the natural N- and

C-termini amino acids, and the natural N-terminal Met may be optionally deleted. In some embodiments, the UGT having 1-2′ glycosylating activity comprises the amino acid sequence of SEQ ID NO:45, or an enzyme having at least about 50% identity, at least about 60% identity, at least about 70% identity, at least about 80% identity, at least about 85% identity, or at least about 90% identity, or at least about 95% identity, or at least 96%, 97%, 98% or 99% identity to SEQ ID NO:45, and having 1-2′ glycosylating activity at one or more of C19 of RebA or C13 of steviolmonoside.

In some embodiments, the UGT enzyme having 1-3′ glycosylating activity is SrUGT76G1, or derivative thereof having the same or increased glycosylation activity at C19 of RebD or C13 of stevioside. In some embodiments, the UGT enzyme having 1-3″ glycosylating activity is a derivative of SrUGT76G1 that includes an amino acid substitution at one or more of positions 77, 78, 81, 82, 93, 94, 155, 192, 200, 202, 205, 283, 284, 379, and 397 of SEQ ID NO: 3 (see Table 13). In some embodiments, the derivative has an amino acid substitution at position L200 (numbered according to the wild type enzyme), and which is optionally Ala or Gly. In these embodiments, the derivative may further have an amino acid substitution at position 284 (e.g., Ala) and/or 379 (e.g., Gly), and/or 192 (e.g., Ala). In some embodiments, an Ala is inserted or substituted at position 2 to decrease turnover in the cell. In some embodiments, the UGT enzyme has at least about 80% identity, at least about 85% identity, at least about 90% identity, or at least about 95%, 96%, 97%, 98%, or 99% identity to SEQ ID NO: 3, with the proviso that the amino acid corresponding to position 200 of SEQ ID NO:3 is Ala or Gly. As shown in Table 13, the substitution of L200A or L200G provides for large improvements in activity at both C19 and C13.

Additional modification to UGT76G1 include modification at one or more of positions 22, 25, 145, 154, 256, and 282, such as one or more of Q22G, Q22H, 125F, 125W, T145A. T145G, T145P, H154R. L256P. L256W, L256T, L256G, L256A, L256R. L256E, S281G and S282N. These modifications are disclosed in WO 2014/122227, which is hereby incorporated by reference. In some embodiments, these additional modifications to UGT76G1 exhibit superior properties in combination with the modifications at positions 77, 78, 81, 82, 83, 93, 94, 155, 192, 200, 202, 205, 283, 284, 378, 379, and 397.

In some embodiments, the UGT enzyme having 1-3′ glycosylating activity is a circular permutant of SrUGT76G1, with a cut-site corresponding to a position within amino acids 170 to 290 (e.g. 190-210, 196-200 or 260-280) of SrUGT76G1. In some embodiments, the cut site corresponds to position 196 or 264 of the wild-type enzyme. The circular permutant (e.g., MbUGT1-3), may have from 1 to 30, or from 1 to 20, or from 1 to 10, or from 1 to 5 amino acid substitutions, deletions, and/or insertions with respect to the corresponding position of the wild-type sequence. In some embodiments, the UGT having 1-3′ glycosylating activity comprises the amino acid sequence of SEQ ID NO: 10 (MbUGT1-3), or comprises an amino acid sequence having at least about 50% identity, at least about 60% identity, at least about 70% identity, at least about 80% identity, at least about 85% identity, or at least about 90% identity. or at least about 95% identity, or at least 96%, 97%, 98% or 99% identity to SEQ ID NO: 10, and having 1-3′ glycosylating activity at one or more of C19 of RebD or C13 of stevioside. In some embodiments, Ala is substituted or inserted at position 2 to decrease turnover in the cell.

In various embodiments, the host cell or method of the invention further involves a UGT enzyme that converts steviol to steviolmonoside. In some embodiments, the UGT enzyme that converts steviol to steviolmonoside is SrUGT85C2, or derivative thereof. In some embodiments, the enzyme contains from 1 to about 50, or from 1 to about 20, or from 1 to about 10 amino acid substitutions, deletions, and/or insertions with respect to SEQ ID NO: 1. For example, the derivative may have at least about 65% identity, or at least about 70% identity. or at least about 80% identity to SEQ ID NO: 1, or at least 90% identity to SEQ ID NO: 1, or at least 95%, 96%, 97%, 98%, or 99% identity to SEQ ID NO: 1, while maintaining the same or similar activity for converting steviol to steviolmonoside (e.g., in vitro or in vivo). Exemplary amino acid modifications are shown in Table 11. In some embodiments, the enzyme that converts steviol to steviolmonoside is a derivative of SrUGT85C2 having an amino acid substitution at position 215 of the wild type enzyme. In some embodiments, the amino acid at the position corresponding to 215 of the wild type enzyme is threonine, serine, glycine, or alanine (the wild type amino acid is Proline). In some embodiments, the amino acid at said position 215 is threonine. In these or other embodiments, the derivative of SrUGT85C2 has a mutation at one or more of positions 308, 311, 316, 349, and/or 414 (numbered in accordance with the wild type enzyme. In some embodiments, the amino acid at position 308 is threonine, and/or the amino acid at position 311 is glutamine, and/or the amino acid at position 316 is alanine, and/or the amino acid at position 349 is glutamic acid, and/or the amino acid at position 414 is glycine. In some embodiments, an Ala is inserted or substituted at the second position to limit turnover in the cell.

In various embodiments, the host cell or method further involves a UGT enzyme that converts steviolbioside to stevioside, which in some embodiments is SrUGT74G1, or derivative thereof. In some embodiments, the enzyme contains from 1 to about 50, or from 1 to about 20, or from 1 to 10 amino acid substitutions, deletions, and/or insertions with respect to SEQ ID NO: 2. For example, the derivative may have at least 80% identity to SEQ ID NO: 2, at least 90% identity to SEQ ID NO: 2, or at least 95%, 96%, 97%, 98%, or 99% identity to SEQ ID NO: 2, while maintaining the same or similar activity for converting steviolbioside to stevioside (e.g., in vitro or in vivo).

In some embodiments, the UGT enzyme that converts steviolbioside to stevioside is a circular permutant of SrUGT74G1 (e.g., MbUGTC19). In some embodiments, the circular permutant has a cut site corresponding to an amino acid within positions 180 to 280 (e.g., 250) to 270) of SrUGT74G1. The circular permutant may have a linking sequence between the original N- and C-termini of from 1 to 10 amino acids (e.g., GSG). The circular permutant may have from 1 to 30, or from 1 to 20, or from 1 to 10, or from 1 to 5 amino acid substitutions, deletions, and/or insertions with respect to the corresponding position of the wild-type sequence. In some embodiments, the SrUGT74G1 circular permutant comprises the amino acid sequence of SEQ ID NO: 8 (MbUGTC19) or SEQ ID NO: 46 (MbUGTC19-2), or comprises an amino acid sequence having at least about 50% identity, at least about 60% identity, at least about 70% identity, at least about 80% identity, at least about 85% identity, or at least about 90% identity, or at least about 95% identity, or at least 96%, 97%, 98% or 99% identity to SEQ ID NO: 8 or 46, and having activity for converting steviolbioside to stevioside.

In some embodiments, the host cell produces steviol substrate through one or more pathway modules comprising a kaurene synthase (KS), kaurene oxidase (KO), and a kaurenoic acid hydroxylase (KAH), the host cell further comprising a cytochrome P450 reductase (CPR) for regenerating one or more of the KO and KAH enzymes. In some embodiments, the KAH is KAH of Stevia rebaudiana, Arabidopsis thaliana, Vitis vinifera, or Medicago trunculata, or a derivative thereof (e.g., having at least 80%, or at least 90%, or at least 95%, or at least 97% sequence identity to the wild type sequence). In some embodiments, the KAH is an Arabidopsis thaliana KAH (AtKAH), or derivative thereof. The AtKAH may have one or more amino acid substitutions, insertions, and/or deletions that increase the rate of kaurenoic acid hydroxylase activity or otherwise improve enzyme productivity or expression, including for example an N-terminus engineered for functional expression in E. coli. In some embodiments, the AtKAH has an amino acid substitution at one or more positions (e.g., two-ten positions) of the parent sequence of SEQ ID NO:29 as shown in Table 6 that increases production of steviol or kaurenoic acid. Exemplary substitutions include substitutions corresponding to the following positions of SEQ ID NO:29: 25 (e.g., A25L), 79 (e.g., S79T), 119 (e.g., T119C), 137 (e.g., I137L), 142 (e.g., 1142V), 155 (e.g., R155K), 180 (e.g., M180L), 193 (e.g., E193G), 196 (e.g. C196A), 197 (e.g., D197E), 226 (A226E), 235 (e.g., L235Q), 238 (e.g., 1238M), 245 (F245L, F245V), 272 (e.g., L272I), 285 (e.g., 1285R), 287 (e.g., C287S), 325 (e.g., C325I, C325M), 330 (e.g., F330L), 334 (e.g., D334E), 339 (e.g., S339T), 352 (e.g., S352E), 373 (e.g., E373D), 397 (e.g., 1397F), 470 (e.g., V470L), 499 (e.g., Q499V), 506 (e.g., L506M), 507 (e.g., L507I, L507T, L507V). In some embodiments, the AtKAH enzyme is a derivative having an amino acid substitution at position 331 (with respect to the wild type sequence), which in some embodiments, improves the productivity of the enzyme at higher temperatures (e.g., higher than 22° C.). In some embodiments, the amino acid at position 331 is Ile.

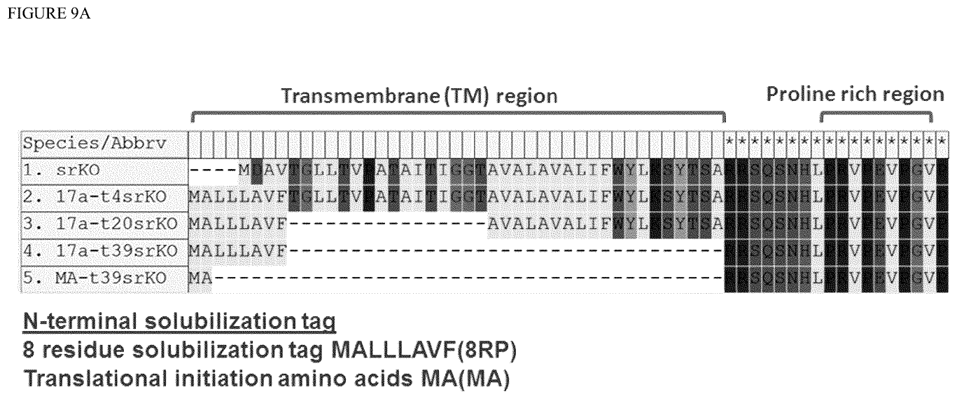

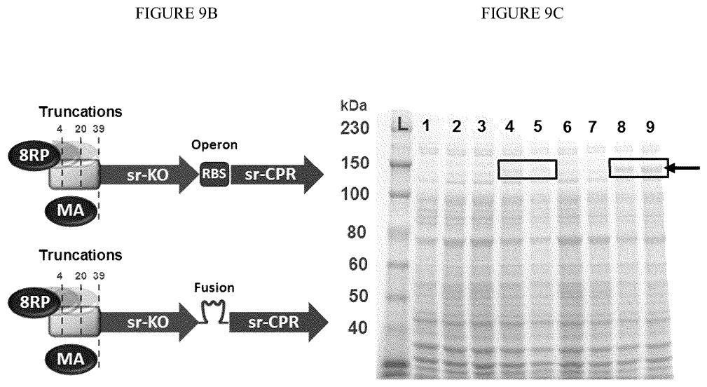

N-terminal modifications to achieve functional expression of the P450 enzyme SrKO are illustrated in FIG. 9A, FIG. 9B, and FIG. 9C. These modifications or similar modifications may be made to achieve functional expression of KAH, including AtKAH. For example, all or portions of the transmembrane region may be deleted, such as from 4 amino acids to about 39 amino acids, or in some embodiments, from about 6 amino acids to about 25 amino acids, or about 4 to about 20 amino acids, or about 29 amino acids, or about 39 amino acids. The deletions are preferably taken from the N-terminal portion of the transmembrane region. This portion is replaced with a solubilization tag of from about 4 to about 20 amino acid residues, such as from about 4 to about 12 residues (e.g., eight amino acid residues). The tag is constructed predominantly of hydrophobic amino acids, which are optionally selected from Ala, Leu, Ile, Val, and Phe. An exemplary sequence for the functional expression is the N-terminal tag: MALLLAVF (SEQ ID NO: 47). In some embodiments, the AtKAH has a truncation of 14 amino acids, with the addition of the N-terminal tag (e.g., SEQ ID NO: 29), optionally having the substitution C331I (position nomenclature based on the wild type enzyme).

Alternative N-terminal tag sequences for P450 enzymes are described in Provisional Application No. 62/208,166, filed Aug. 21, 2015, and which find use in certain embodiments of the present invention. For example, the transmembrane domain (or “N-terminal anchor”) can be derived from an E. coli gene selected from waaA, ypfN, vhcB, yhbM, yhhm, zipA, ycgG, dj1A, sohB, lpxK, F11O, motA, htpx, pgaC, ygdD, hemr, and ycls. These genes were identified as inner membrane, cytoplasmic C-terminus proteins through bioinformatic prediction as well as experimental validation. The invention may employ an N-terminal anchor sequence that is a derivative of the E. coli wild-type transmembrane domain, that is, having one or more mutations with respect to the wild-type sequence. In exemplary embodiments, the membrane anchor sequence is from about 8 to about 75 amino acids in length. For example, the membrane anchor may be from about 15 to about 50, or from about 15 to about 40, or from about 15 to about 30, or from about 20 to about 40), or from about 20 to about 30 amino acids in length.

In some embodiments, the Kaurene Synthase (KS) is from Stevia rebaudiana, Zea mays, Populus trichocarpa, Arabidopsis thaliana, Erwina tracheiphila or derivative thereof (e.g., having at least 80%, or at least 90%, or at least 95%, or at least 97% sequence identity to the wild type sequence). Further, the cell may express a copalyl diphosphate synthase (CPPS) from Stevia rebaudiana, Streptomyces clavuligerus, Bradyrhizobium japonicum, Zea mays, Arabidopsis thaliana, Erwina tracheiphila, or derivative thereof (e.g., having at least 80%, or at least 90%, or at least 95%, or at least 97% sequence identity to the wild type sequence). In some embodiments, the host cell expresses a bifunctional CPPS and KS enzyme, which is optionally selected from Phomopsis amygdali, Physcomitrella patens, Gibberella fujikuroi enzyme, or derivative thereof. Such derivative may generally have at least about 75%, or at least about 80%, or at least about 85%, or at least about 90%, or at least about 95%, or at least 96%, 97%, 98%, or 99% identity to the parent sequence (e.g., see Table 1). In some embodiments, the cell expresses Erwina tracheiphila CPPS and KS enzymes, or derivatives thereof.

In some embodiments, the host cell expresses a Kaurene Oxidase from Stevia rebaudiana (SrKO), Arabidopsis thaliana, Gibberella fujikoroi, or Trametes versicolor, or a derivative thereof, which is optionally modified at the N-terminus for functional expression in E. coli (as described above and as shown in FIG. 9A, FIG. 9B, and FIG. 9C). In some embodiments, the CPR is an enzyme of Stevia rebaudiana (SrCPR), Arabidopsis thaliana, or Giberella fujikuroi, or a derivative thereof, which is optionally modified at the N-terminus for functional expression in E. coli.

The SrKO may have one or more amino acid modifications that improve its activity. Exemplary modifications are disclosed in U.S. Provisional Application No. 62/040,284, which is hereby incorporated by reference in its entirety. For example, the SrKO may comprise one or more amino acid modifications at positions (with respect to SEQ ID NO:22: 47 (e.g., L47I), 59 (e.g., Y59H), 60 (e.g., M60K), 63 (e.g., T63A), 67 (e.g., A67E), 76 (e.g., K76R), 80 (e.g., T80C), 82 (e.g., M82V), 85 (e.g., V85L, V851), 86 (e.g., S86N), 100 (e.g., Q100S), 106 (e.g., N106K), 112 (e.g., K112T), 116 (A116R), 119 (e.g., T119S), 123 (e.g., M123T, M123Q, M123F, M123T), 127 (e.g., D127G), 129 (e.g., Y129F), 140 (e.g., A140R), 149 (e.g., K149R), 150 (e.g., H150F), 171 (e.g., N171D), 180 (e.g., L180F), 183 (e.g., 1183V), 208 (e.g., D208E), 232 (e.g., D232E), 267 (e.g., S267A), 272 (e.g., H272Q), 284 (e.g., S284C), 286 (e.g., 1286L), 294 (e.g., Q294K), 299 (e.g., Q299E), 310 (e.g., 1310T, 1310V), 371 (e.g., R371K, R371I), 375 (e.g., V375T, V375I, V375L), 378 (e.g., 1378V), 382 (e.g., H382Y), 388 (e.g., V388Q, V388M), 393 (e.g., H393D), 400 (e.g., L400I), 413 (e.g., V413K, V413D), 434 (e.g., F434L), 442 (e.g., G442A), 450 (e.g., S450A), 454 (e.g., L454M), 460 (e.g., G460A), 464 (e.g., M464L), 475 (e.g., M475G), 487 (e.g., T487N), 492 (e.g., P492K), and 497 (e.g., 1497L). In some embodiments, the SrKO contains a truncation of about 20 amino acids of the N-terminal transmembrane domain, with addition of an N-terminal tag sequence (described above). The SrKO may contain an Ala at the 2nd position to decrease enzyme turnover in the cell.

In some embodiments, the P450 reductase partner(s) include Stevia rebaudiana (Sr)CPR, Stevia rebaudiana (Sr)CPR1, Arabidopsis thaliana (At)CPR, Taxus cuspidata (Tc)CPR, Artemisia annua (Aa)CPR, Arabidopsis thaliana (At)CPR1, Arabidopsis thaliana (At)CPR2, Arabidopsis thaliana (At)R2, Stevia rebaudiana (Sr)CPR2, Stevia rebaudiana (Sr)CPR3, or Pelargonium graveolens (Pg)CPR. Any of these P450s can be derivatized in some embodiments, for example, to introduce from 1 to about 20 mutations, or from about 1 to about 10 mutations. These CPR proteins are further described in PCT/US15/46369, which disclosure is hereby incorporated by reference.

In some embodiments, the host cell is an E. coli that contains a single CPR enzyme (e.g., SrCPR), and which is chromosomally integrated, and supports both the SrKO and AtKAH enzymes, for example.

In some embodiments, the host cell expresses a geranylgeranyl pyrophosphate synthase (GGPPS), which is optionally of Taxus canadensis, Abies grandis, Aspergillus nidulans, Stevia rebaudiana, Gibberella fujikuroi, Mus musculus, Thalassiosira pseudonana, Streptomyces melanosporofaciens, Streptomyces clavuligerus, Sulfulubus acidocaldarius, Synechococcus sp. (e.g., JA-3-3Ab). Arabidopsis thaliana, Marine bacterium 443, Paracoccus haeundaensis. Chlorobium tepidum TLS, Synechocystis sp. (PCC 6803), Thermotoga maritima HB8, Corynebacterium glutamicum. Thermus thermophillus HB27, Pyrobaculum calidifontis JCM 11548, or derivative thereof. See Table 1. Such derivative may generally have at least about 60%, or at least about 70%, or at least about 75%, or at least about 80%, or at least about 85%, or at least about 90%, or at least about 95%, or at least 96%, 97%, 98%, or 99% identity to the parent sequence (e.g., see Table 1). In some embodiments, the GGPPS is Taxus canadensis or derivative thereof. In some embodiments, the Taxus GGPPS is an N-terminal truncated sequence (e.g., with the N-terminal 70 to 110, such as about 98, amino acids truncated). The truncated sequence may further comprise from about 1 to about 10, such as from about 1 to about 5 amino acid substitutions, deletions, and/or insertions at the corresponding wild-type sequence. An exemplary truncated sequence is disclosed herein as SEQ ID NO: 12. In some embodiments, the GGPPS is from Cornybacterium glutamicum or derivative thereof, which can provide advantages in productivity at temperatures higher than 22° C.

In some embodiments, the host cell expresses a pathway producing iso-pentyl pyrophosphate (IPP) and dimethylallyl pyrophosphate (DMAPP). In some embodiments, the pathway is a methylerythritol phosphate (MEP) pathway and/or a mevalonic acid (MVA) pathway.

The MEP (2-C-methyl-D-erythritol 4-phosphate) pathway, also called the MEP/DOXP (2-C-methyl-D-erythritol 4-phosphate/l-deoxy-D-xylulose 5-phosphate) pathway or the non-mevalonate pathway or the mevalonic acid-independent pathway refers to the pathway that converts glyceraldehyde-3-phosphate and pyruvate to IPP and DMAPP. The pathway typically involves action of the following enzymes: 1-deoxy-D-xylulose-5-phosphate synthase (Dxs), 1-deoxy-D-xylulose-5-phosphate reductoisomerase (IspC), 4-diphosphocytidyl-2-C-methyl-D-erythritol synthase (IspD), 4-diphosphocytidyl-2-C-methyl-D-erythritol kinase (IspE), 2C-methyl-D-erythritol 2.4-cyclodiphosphate synthase (IspF), 1-hydroxy-2-methyl-2-(E)-butenyl 4-diphosphate synthase (IspG), and isopentenyl diphosphate isomerase (IspH). The MEP pathway, and the genes and enzymes that make up the MEP pathway, are described in U.S. Pat. No. 8,512,988, which is hereby incorporated by reference in its entirety. For example, genes that make up the MEP pathway include dxs, ispC, ispD, ispE, ispF, ispG, ispH, idi, and ispA. In some embodiments, steviol is produced at least in part by metabolic flux through an MEP pathway, and wherein the host cell has at least one additional copy of a dxs, ispD, ispF, and/or idi gene. As disclosed in U.S. Pat. No. 8,512,988, the level of the metabolite indole can be used as a surrogate marker for efficient production of terpenoid products in E. coli through the MEP pathway.

The MVA pathway refers to the biosynthetic pathway that converts acetyl-CoA to IPP. The mevalonate pathway typically comprises enzymes that catalyze the following steps: (a) condensing two molecules of acetyl-CoA to acetoacetyl-CoA (e.g., by action of acetoacetyl-CoA thiolase): (b) condensing acetoacetyl-CoA with acetyl-CoA to form hydroxymethylglutaryl-CoenzymeA (HMG-COA) (e.g., by action of HMG-COA synthase (HMGS)): (c) converting HMG-COA to mevalonate (e.g., by action of HMG-COA reductase (HMGR)): (d) phosphorylating mevalonate to mevalonate 5-phosphate (e.g., by action of mevalonate kinase (MK)): (e) converting mevalonate 5-phosphate to mevalonate 5-pyrophosphate (e.g., by action of phosphomevalonate kinase (PMK)); and (f) converting mevalonate 5-pyrophosphate to isopentenyl pyrophosphate (e.g., by action of mevalonate pyrophosphate decarboxylase (MPD)). The MVA pathway, and the genes and enzymes that make up the MEP pathway, are described in U.S. Pat. No. 7,667,017, which is hereby incorporated by reference in its entirety.

The host cell may be prokaryotic or eukaryotic. For example, the host cell may be a bacteria selected from E. coli, Bacillus subtillus, or Pseudomonas putida. In some embodiments, the host cell is a species of Saccharomyces, Pichia, or Yarrowia, including Saccharomyces cerevisiae, Pichia pastoris, and Yarrowia lipolytica. The host cell may be an E. coli having a duplication or overexpression of dxs, idi, IspD, and IspF increasing production of IPP and DMAPP.

In some embodiments, the host cell is an E. coli having one or more genetic modifications increasing the production of UDP-glucose, for example, increasing UDP-glucose substrate availability. To improve availability of UDP-glucose for steviol glycosylation, a series of gene knock-outs and gene insertions can be introduced to increase carbon flux to UDP-glucose and decrease flux in pathways away from UDP-glucose (e.g., glycogen synthesis and carbon storage). For example, genetic modifications can increase importation of sucrose into the cell and split it into fructose and glucose via the activity of sucrose phosphorylase. A subsequent series of knock-outs can alter primary metabolism so as to force biomass to be synthesized using only fructose as carbon source, leaving glucose to be funneled exclusively towards UDP-glucose biosynthesis. Exemplary modifications to an E. coli strain to enact this strategy are listed in Table 7. Modifications are further described in PCT/EP2011/061891, which is hereby incorporated by reference in its entirety. In some embodiments, the one or more genetic modifications include ΔgalE, ΔgalT, ΔgalK, ΔgalM, ΔushA, Δagp, Δpgm, duplication of E coli GALU, and expression of Bacillus substillus UGPA, BaSP.

In an exemplary embodiment, the host cell is an E. coli that comprises the following heterologously expressed genes: Taxus canadensis GGPPS or derivative thereof, Phaeosphaeria sp. PsCK or derivative thereof, Stevia rebaudiana KO or derivative thereof, Arabidopsis thaliana KAH or derivative thereof, Stevia rebaudiana CPR or derivative thereof (and which is the only CPR enzyme expressed by the host cell), Stevia rebaudiana UGT85C2 or derivative thereof, Stevia rebaudiana UGT74G1 of derivative thereof, Stevia rebaudiana UGT76G1 or derivative thereof, and MbUGT1-2 or derivative thereof. Various derivatives of these enzymes are disclosed herein. In some embodiments, the E. coli contains a polycistronic expression module of KAH-KO, and contains a single copy of SrCPR (or derivative) that is chromosomally integrated. In some embodiments, the E. coli is modified to increase availability of UDP-glucose as described above. In some embodiments, the E. coli has an additional copy of dxs, idi, ispD, and ispF genes. In some embodiments, one or more expressed proteins contain an Alanine at position 2, to provide additional stability in vivo.

In other embodiments, the host cell is an E. coli that comprises the following heterologously expressed genes: Cornybacterium glutamicum GGPPS or derivative thereof; Erwina tracheiphila CPPS and KS or derivative of one or both: Stevia rebaudiana KO or derivative thereof: Arabidopsis thaliana KAH or derivative thereof; a Stevia rebaudiana CPR or derivative thereof: Stevia rebaudiana UGT85C2 or MbUGTc13 or derivative thereof: Stevia rebaudiana UGT74G1 or derivative thereof (or MbUGTC19, MbUGTC19-2, or derivative thereof): Stevia rebaudiana UGT76G1 or derivative thereof (or MbUGT1-3 of derivative thereof); and OsUGT1-2, SrUGT91D2, or derivative thereof, or MbUGT1-2 or MbUGT1, 1-2 or derivative thereof. Various derivatives of these enzymes are disclosed herein. In some embodiments, the E. coli contains a polycistronic expression module of KAH-KO, and contains a single copy of SrCPR that is chromosomally integrated. In some embodiments, the E. coli is modified to increase availability of UDP-glucose as described above. In some embodiments. the E. coli has an additional copy of one or more (or all) of dxs, idi, ispD, and ispF genes. In some embodiments, one or more expressed proteins contain an Alanine at position 2, to provide additional stability in vivo. In some embodiments, the E. coli provides increased productivity of Reb M or Reb D at temperatures above about 24° C., such as about 27° C. or more, or about 30° C. or more, or about 32° C. or more, or about 34° C. or more, or about 37° C.

In some embodiments, the method further comprises recovering the desired steviol glycoside(s) (e.g., RebM or RebD) from culture media. In some embodiments, the desired steviol glycoside (e.g., RebM or RebD) is produced in the culture media at a concentration of at least about 10 mg/L, or at least about 100 mg/L, or at least about 200 mg/L, or at least about 500 mg/L, or at least about 1 g/L, or at least about 10 g/L.

Optionally, the method of the present invention further comprises separating the target steviol glycoside from the starting composition. The target steviol glycoside can be separated by any suitable method, such as, for example, crystallization, separation by membranes, centrifugation, extraction, chromatographic separation or a combination of such methods. Fractions containing different glycoside fractions can be blended to prepare defined products. Alternatively, RebM and RebD, for example, can be prepared and purified from separate cultures, and blended at a predetermined ratio.

In another aspect, the invention provides a method for production of steviol glycosides having at least 4 glycosylations in E. coli. In accordance with the invention, the E. coli cell comprises a plurality of UGT enzymes, which may include one or more enzymes described herein, that together perform at least 4, at least 5, or at least 6 (including 7 or 8), sequential glycosylation reactions. As disclosed herein, the glycosylation substrates and lower glycosylation products accumulate in the E. coli cell sufficiently to allow downstream reactions to proceed at an acceptable rate, with a high majority of the glycosylation products ultimately accumulating extracellularly, most likely through the action of a membrane transporter. The steviol glycosides can be purified from media components. Thus, in some embodiments, the methods comprise separating growth media from the E. coli cells, for example using batch, continuous, or semi-continuous bioreactor processes, and isolating the desired glycosylation products (e.g. Reb M) from the growth media.

In still other aspects, the invention provides methods for production of steviol glycosides (including Reb D, Reb M, Reb E, Reb I and other glycosylation products) in E. coli. Generally, the desired steviol glycoside has at least 2 glycosylations, such as 2, 3, 4, 5, 6, 7, or 8 glycosylations. In some embodiments, the steviol glycoside is RebM or RebD. While many of the enzymes known for production of steviol in host cells are plant enzymes, which often have optimal temperatures in the range of 20-24° C., E. coli growth rate and metabolism are optimal at higher temperatures. The present disclosure enables production of steviol glycosides at high yield in E. coli, by enabling enzyme productivity at temperatures above 24° C., such as from 24° C. to 37° C., or from 27° C. to 37° C., or from 30° C. to 37° C.

While commercial biosynthesis in E. coli can often be limited by the temperature at which overexpressed and/or foreign enzymes are stable, the present disclosure in some embodiments allows for cultures to be maintained at higher temperatures, resulting in higher yields and higher overall productivity. In some embodiments, the culturing is conducted at about 30° C. or greater, or about 31° C. or greater, or about 32° C. or greater, or about 33° C. or greater, or about 34° C. or greater, or about 35° C. or greater, or about 36° C. or greater, or about 37° C.

The host cells and methods are further suitable for commercial production of steviol glycosides, that is, the cells and methods can be productive at commercial scale. In some embodiments, the size of the culture is at least about 100 L, at least about 200 L, at least about 500 L, at least about 1,000 L, or at least about 10,000 L. In an embodiment, the culturing may be conducted in batch culture, continuous culture, or semi-continuous culture.

In some aspects, the invention provides methods for making a product comprising a steviol glycoside ingredient, which is RebM or RebD in some embodiments. The method comprises culturing a strain described herein that produces the steviol glycoside, recovering the steviol glycoside, and incorporating the steviol glycoside into a product, such as a food, beverage, oral care product, sweetener, flavoring agent, or other product.

Purified steviol glycosides, prepared in accordance with the present invention, may be used in a variety of products including, but not limited to, foods, beverages, texturants (e.g., starches, fibers, gums, fats and fat mimetics, and emulsifiers), pharmaceutical compositions, tobacco products, nutraceutical compositions, oral hygiene compositions, and cosmetic compositions. Non-limiting examples of flavors for which RebM can be used in combination include lime, lemon, orange, fruit, banana, grape, pear, pineapple, mango, bitter almond, cola, cinnamon, sugar, cotton candy and vanilla flavors. Non-limiting examples of other food ingredients include flavors, acidulants, and amino acids, coloring agents, bulking agents, modified starches, gums, texturizers, preservatives, antioxidants, emulsifiers, stabilizers, thickeners and gelling agents.

Highly purified target steviol glycoside(s) obtained according to this invention may be incorporated as a high intensity natural sweetener in foodstuffs, beverages, pharmaceutical compositions, cosmetics, chewing gums, table top products, cereals, dairy products, toothpastes and other oral cavity compositions, etc.

Highly purified target steviol glycoside(s) obtained according to this invention can be used in combination with various physiologically active substances or functional ingredients. Functional ingredients generally are classified into categories such as carotenoids, dietary fiber, fatty acids, saponins, antioxidants, nutraceuticals, flavonoids, isothiocyanates, phenols, plant sterols and stanols (phytosterols and phytostanols): polyols: prebiotics, probiotics: phytoestrogens: soy protein: sulfides/thiols: amino acids: proteins: vitamins; and minerals. Functional ingredients also may be classified based on their health benefits, such as cardiovascular, cholesterol-reducing, and anti-inflammatory.

Highly purified target steviol glycoside(s) obtained according to this invention may be applied as a high intensity sweetener to produce zero calorie, reduced calorie or diabetic beverages and food products with improved taste characteristics. It may also be used in drinks, foodstuffs, pharmaceuticals, and other products in which sugar cannot be used. In addition, highly purified target steviol glycoside(s), particularly, RebM can be used as a sweetener not only for drinks, foodstuffs, and other products dedicated for human consumption, but also in animal feed and fodder with improved characteristics.

Examples of products in which highly purified target steviol glycoside(s) may be used as a sweetening compound include, but are not limited to, alcoholic beverages such as vodka, wine, beer, liquor, and sake, etc.; natural juices; refreshing drinks; carbonated soft drinks; diet drinks; zero calorie drinks; reduced calorie drinks and foods; yogurt drinks; instant juices; instant coffee; powdered types of instant beverages; canned products; syrups; fermented soybean paste; soy sauce; vinegar; dressings; mayonnaise; ketchups; curry; soup; instant bouillon; powdered soy sauce; powdered vinegar; types of biscuits; rice biscuit; crackers; bread; chocolates; caramel; candy; chewing gum; jelly; pudding; preserved fruits and vegetables; fresh cream; jam; marmalade; flower paste; powdered milk; ice cream; sorbet; vegetables and fruits packed in bottles; canned and boiled beans; meat and foods boiled in sweetened sauce; agricultural vegetable food products; seafood; ham; sausage; fish ham; fish sausage; fish paste; deep fried fish products; dried seafood products; frozen food products; preserved seaweed; preserved meat; tobacco; medicinal products; and many others. In principle it can have unlimited applications.

During the manufacturing of products such as foodstuffs, drinks, pharmaceuticals, cosmetics, table top products, and chewing gum, the conventional methods such as mixing, kneading, dissolution, pickling, permeation, percolation, sprinkling, atomizing, infusing and other methods may be used.

### Examples

Steviol glycosides are the natural constituents of the plant Stevia rebaudiana, known commonly as Stevia. Steviol glycoside Rebaudioside M (RebM) (FIG. 1), whose taste profile drastically improves upon that of other steviol glycosides, is an ideal candidate to replace currently used steviol glycosides such Rebaudioside A, but hasn't fulfilled that promise because of its low levels in the Stevia plant (<0.01%). Steviol is a diterpenoid that forms the core chemical structure of steviol glycosides like RebM (1).

Terpenoid biosynthesis has been engineered in both prokaryotic (e.g., E. coli) and eukaryotic (e.g., yeast) cells for heterologous production of complex terpenoid molecules (2,3). The E. coli MEP-pathway is stoichiometrically superior and less byproduct accumulating compared to the yeast MVA-pathway (4,5). A new metabolic engineering approach, multivariate modular metabolic engineering (MMME), and a platform E. coli strain capable of overproducing terpenoid precursors has been described (4,6). MMME facilitates assessment and elimination of regulatory and pathway bottlenecks by re-defining the metabolic network as a collection of distinct modules (7). By grouping enzymes with similar turnovers into a subset, or module, and later equalizing the turnover of the different subsets by adjusting concentrations/activities, one can maximize the ratio of pathway turnover to resource expenditure.

MMME pathway engineering was applied in E coli for the biosynthesis of kaurene, the unfunctionalized terpene scaffold precursor for steviol and steviol glycosides. Next, the downstream CYP450-mediated oxidation chemistry was engineered to demonstrate that the diterpenoid scaffold steviol can be biosynthesized in E. coli. Further, glycosylation chemistry for the conversion of steviol to steviol glycosides in E. coli was engineered to develop a technology platform for producing glycosylated natural products. Further still, E. coli were engineered to produce improved levels of UDP-glucose to support high levels of steviol glycoside production. This work provides for an economical, commercially-viable source for RebM (and other steviol glycosides described herein) in microbial systems from renewable resources.

### Example 1: Biosynthesis of Steviol and Steviol Glycosides

Steviol glycosides are diterpenoid derivatives and their early biosynthetic pathways share common intermediates with gibberellic acid biosynthesis (8). The overall linear pathway is modularized into four parts: (1) the formation of starting precursor IPP and DMAPP from the central carbon metabolites derived from glucose, (2) the production of the first dedicated intermediate, kaurene: (3) biosynthesis of the key intermediate, steviol; and (4) the formation of various steviol glycosides. A further module (5) is independently engineered to support the increased production of UDP-glucose, the second substrate necessary for glycosylation of steviol. The five modules are shown in FIG. 2.

In plants, the formation of common isoprenoid precursors IPP and DMAPP can be derived from two biosynthetic routes: either the mevalonic acid (MVA) pathway or methylerythritol-phosphate (MEP) pathway (9). The first step in steviol diterpenoid biosynthesis is conversion of IPP and DMAPP into geranyl-geranyl diphosphate (GGPP). GGPP is the four subunit precursor for all diterpenoid molecules. Next, protonation-initiated cyclization of GGPP to copalyl diphosphate (CPP) is catalyzed by CPP synthase (CPPS). Kaurene is then produced from CPP by an ionization-dependant cyclization catalyzed by kaurene synthase (KS). These enzymes have been identified and characterized from the native biosynthetic pathway in Stevia. In addition to this, there are bi-functional enzymes characterized from the basal plant (Physcomitrella patens) and fungal species (e.g., Gibberella fujikuroi and Phaeosphaeria sp.) for conversion of GGPP into kaurene (10,11). Kaurene is then oxidized in a three-step reaction to kaurenoic acid, by kaurene oxidase (KO) a P450 mono-oxygenase. A full length KO cDNA was expressed in yeast and demonstrated that it could convert kaurene to kaurenoic acid (12). The next step in the pathway is the hydroxylation of kaurenoic acid by kaurenoic acid 13-hydroxylase (KAH). KAH, a cytochrome P450, was expressed in yeast and converted kaurenoic acid to steviol (13).

With the core steviol molecule assembled, a series of six glycosylations attach six glucose moieties to the steviol core. The glycosyltransferase enzymes (EC 2.4.1.17) responsible for these activities catalyze the transfer of the glucose component of UDP-glucose to a small hydrophobic molecule, in this case the steviol molecule (14). O-glycosylations occur at the C13 and C19 positions of steviol (FIG. 1), followed by 1-2′ glycosylations and 1-3′ glycosylations at both these O-glucosyls to result in six glycosylations in total. The order of glycosylations can be quite complex, with various intermediate products forming given variation in the order of C13 or C19 glycosylations, as well as 1-2′ or 1-3′ glycosylations (FIG. 3). Given the intermediate product pools accumulating in Stevia rebaudiana, a potential pathway for the production of RebM is Steviol>Steviolmonoside>Steviolbioside>Stevioside>Rebaudioside A>Rebaudioside D>Rebaudioside M. However, this does not preclude an alternate pathway in microbial biosynthetic systems (FIG. 3).

Detailed understanding and characterization of biochemical pathways for steviol glycosides and advancements in engineering of the upstream isoprenoid pathway to reroute the IPP and DMAPP through heterologous biosynthetic pathway engineering provides the basis for directed, sustainable production of purified and high quality steviol glycosides in a convenient microbial-based bioprocess. The current plant-based production and purification schemes present significant challenges to reducing costs. The microbial route described herein using plant pathways that have been reconstructed in microbial hosts offers superior opportunities for improving current processes and to generate superior quality steviol glycosides that are of very low abundance in nature.

(A) Engineering Kaurene Biosynthesis in E. coli

Kaurene is the cyclic diterpenoid precursor for steviol and plant growth hormone gibberellic acid. The biosynthesis of kaurene consists of three steps from the universal terpenoid precursor IPP and DMAPP. The three step reaction from IPP and DMAPP is catalyzed by enzymes GPPS. CPS and KS or bifunctional CPPS/KS enzymes. The overall pathway up to kaurene is grouped as two modules (FIG. 2). There have been several enzymes from different organisms characterized for the conversion of IPP and DMAPP to GGPP (15-18) and GGPP to kaurene (9-12.18) and kaurene to steviol (12.19-24) (Table 1). In higher plants, such as stevia. GGPP to kaurene biosynthesis is carried out as two step reaction mediated by enzymes called copalyl pyrophosphate synthase (CPPS) and kaurene synthase (KS). In the basal plant (Physcomitrella patens) and fungal (e.g., Gibberella fujikuroi and Phaeosphaeria sp.) species. the GGPP to kaurene biosynthesis is carried out by bi-functional enzymes characterized in these organisms. Similarly. there are multiple enzymes cloned and characterized as converting IPP and DMAPP to make GGPP. The first step towards engineering kaurene biosynthesis is therefore selection of enzymes. Enzymes from different species were selected to test for biosynthesis of kaurene (Table 1). Studies on MMME optimization of taxadiene biosynthesis show that the kinetics of TcGPPS are capable of supporting ˜1 g/L production of taxadiene and therefore other diterpenes. To identify the best kaurene synthase ortholog. TcGPPS was selected as the upstream candidate enzyme. Operons were then selected containing KS-CPS-GGPPS (KCG) or bi-functional PsCK-GGPPS (CKG) to test the pathway in the upstream pathway engineered strains. To modulate the expression of the downstream kaurene pathway. the KS-CPPS-GGPPS (KCG) and CK-GGPPS (CKG) operons were cloned to a plasmid system with varying copy number and promoter strength (p5Trc. p10Trc. p20Trc and p5T7). Additionally, one copy of each kaurene operon was integrated into the E. coli chromosome under varying promoter strength, coupled with varying upstream pathway expression levels.

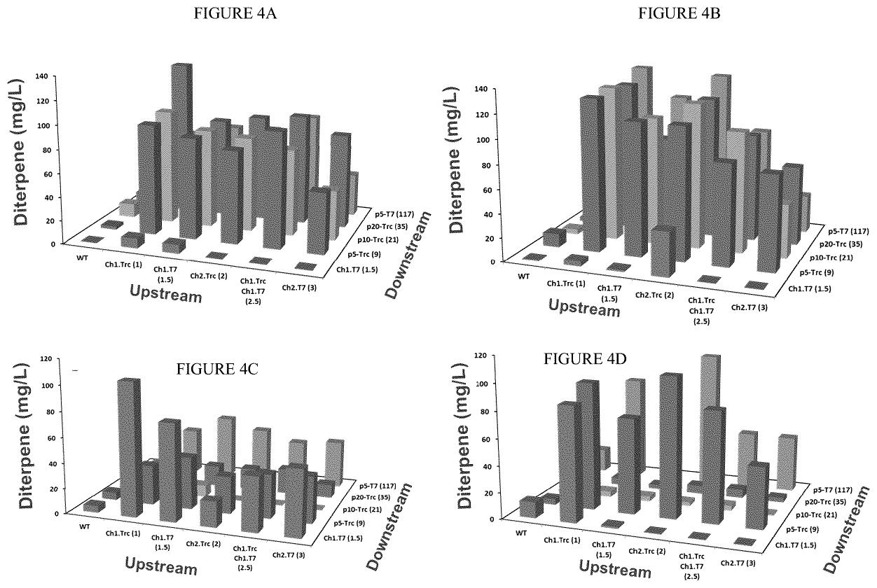

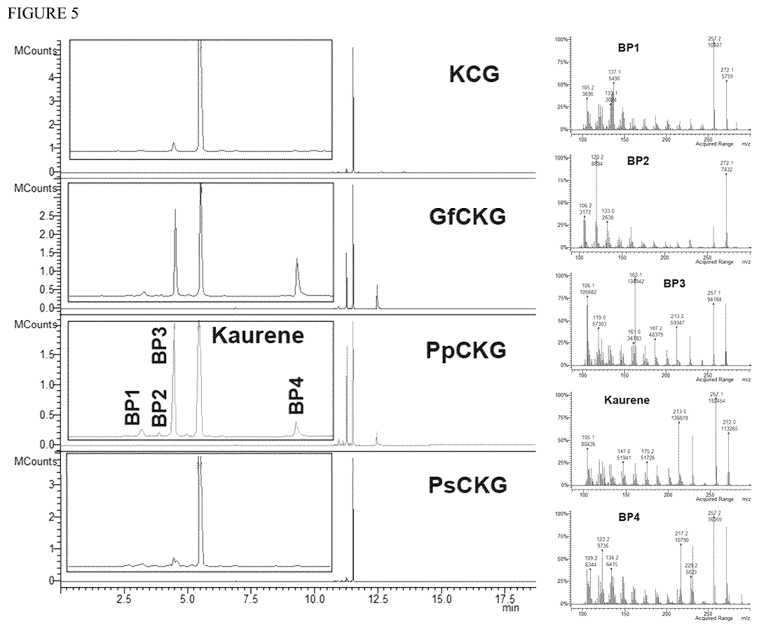

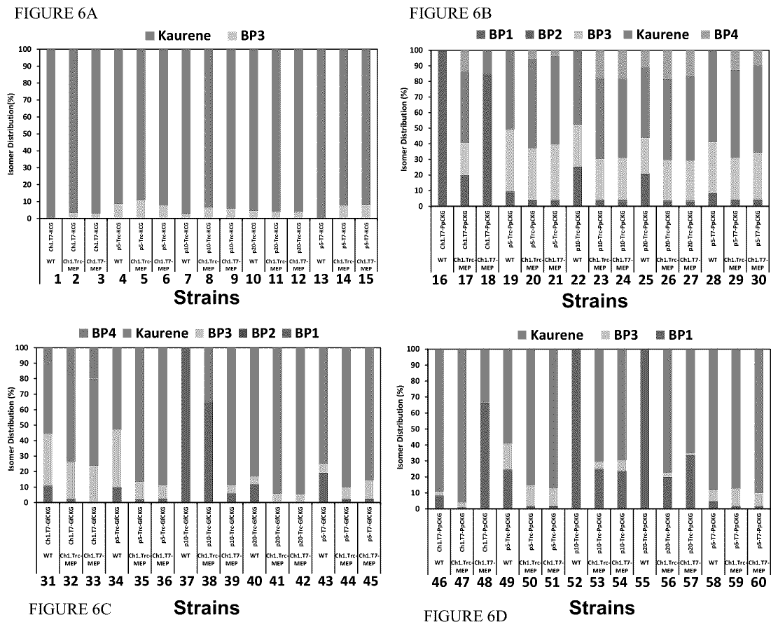

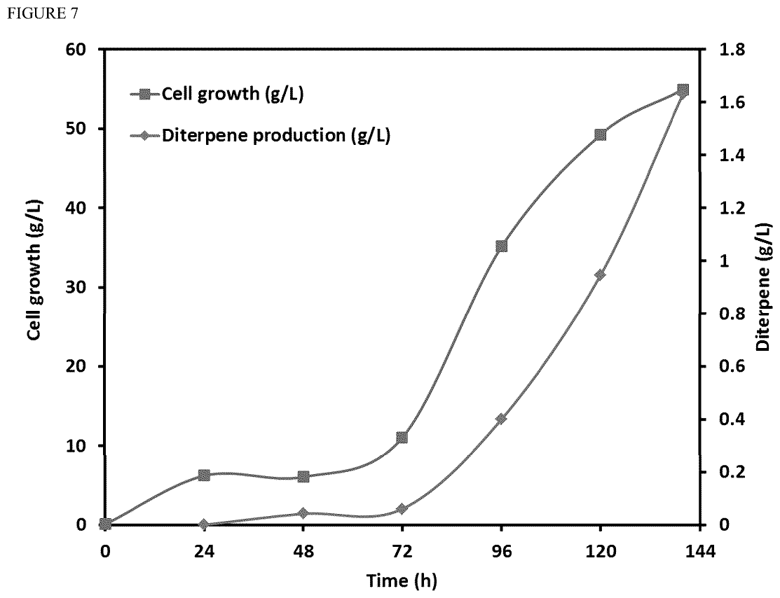

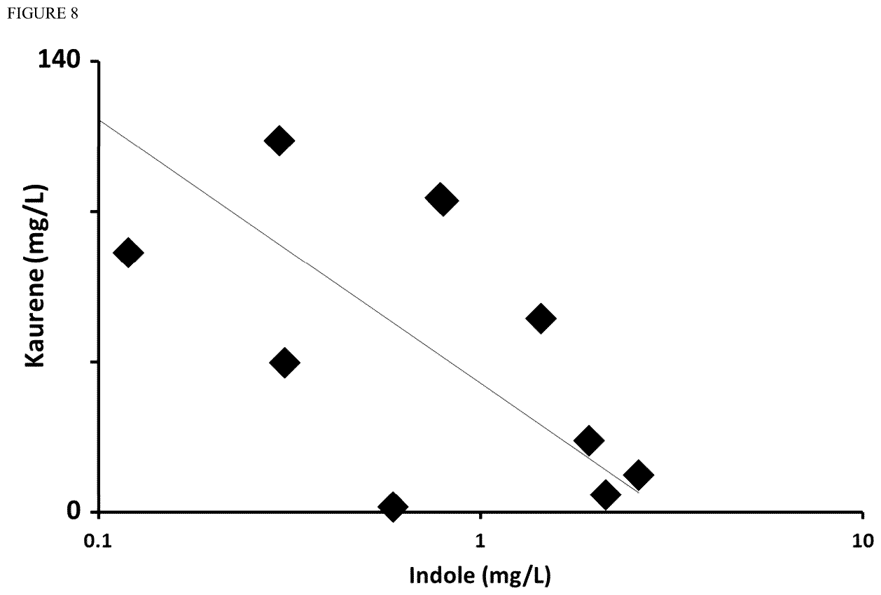

Strains were selected with varying upstream and downstream expression to modulate the pathway and test the productivity of the various combinations. These strains were subjected to small scale (2 mL) Hungate tube fermentation to characterize the phenotypic characteristics and kaurene productivity. As shown in FIG. 4A, FIG. 4B, FIG. 4C, and FIG. 4D, a complex non-linear accumulation of kaurene was observed. Interestingly. KS from plant species (SrCPPS. SrKS and PpCK) showed similar profiles (FIG. 4A, and FIG. 4B). whereas the pathways constructed with fungal enzymes (GfCK and PsCK) showed very similar patterns of product accumulation (FIG. 4C, and FIG. 4D). Interestingly. the low-copy expression of pathways incorporating fungal enzymes showed relatively high productivity compared to the plant enzyme pathways. The global maximum in product titer (˜140 mg/L) comes from a construct with exclusively plant enzymes (FIG. 4A, strain constructed with Stevia rebaudiana genes with upstream under Trc promoter and downstream in plasmid p20Trc). However, the completely chromosomally-integrated fungal pathway enzyme (PsCK) (FIG. 4D, strain with upstream Trc and downstream T7-PsCKG) produced ˜100 mg/L of kaurene. Comparing the expression of the downstream components of these two strains, the Ch1T7PsCKG pathway is 23-fold less (1.5 a.u.) compared to the p20TrcSrKCG (35 a.u.) under the same upstream pathway Ch1TrcMEP strength (4). The key performance driver of a multistep/multi-module pathway is optimal balance in the flux. Here in the strain constructed with Ch1TrcMEP and Ch1T7PsCKG, with very low downstream expression we achieved kaurene production up to 100 mg/L. This demonstrated that the PsCK enzyme can support high flux under balanced pathway expression. In addition. this study also provided insights about the complex non-linear behavior on diterpene product profile under different pathway balance (FIG. 5 and FIG. 6A, FIG. 6B, FIG. 6C, and FIG. 6D. Table 2). Such complex behavior on product selectivity of a pathway under varying flux modulations clearly demonstrates the power of multivariate-modular pathway optimization. Under optimal balance a strain can show high selectivity in product profile (FIG. 6D, strain 47). In addition. the multivariate-modular search allowed selection of the best variant kaurene enzyme (PsCK) to further engineer hyper-producing strains. When this optimal strain (i.e., Strain 47) was grown in a bioreactor system, we were able to—with minimal media or process improvements—generate a strain capable of 1.6 g/L production of kaurene (FIG. 7). MMME also provides insight towards further optimization of the pathway and helps identify the best variant of GGPPS enzyme (Table 1) using a similar approach. Furthermore, as observed in pathway engineering on taxadiene-producing strains. kaurene production also inversely correlated to the production of the inhibitory molecule indole (FIG. 8).

(B) Engineering Steviol Biosynthesis in E. coli

The biosynthesis of steviol involves two key oxidation reactions mediated by cytochrome P450 enzymes (FIG. 3). P450s are important oxidizing enzymes involved in the metabolic pathways of thousands of natural products (25). Until recently. the scientific community believed that when compared to native eukaryotic hosts (e.g. plants or yeast). bacterial hosts, such as E. coli, were not an ideal system for performing this important natural product chemistry. However, while optimizing taxol biochemistry in E. coli, an understanding was developed of the mechanistic structure-function relationships responsible for the biochemistry of P450 enzymes, specifically related to their use in E. coli. Optimal engineering of N-terminal membrane region and construction of optimal combinations of CYP450) and the co-factor P450 reductase (CPR) enzymes is key for functional expression. Several enzyme/pathway optimization techniques were developed for the functional expression CYP450 enzymes and in vivo oxidation of complex natural terpenoid natural products such as taxadiene, valencene, limonene or kaurene.

Steviol biosynthesis is mediated by two different CYP450 enzymes, kaurene oxidase (KO) and kaurenoic acid hydroxylase (KAH) with a CYP450 reductase (CPR). Several candidate genes/enzymes were identified and annotated as P450 enzymes for oxidation and hydroxylation reactions in steviol biosynthesis (Table 1). The functional expression of the enzymes KO and KAH for carboxylation and hydroxylation requires protein redesign and engineering. We started with redesigning and cloning the SrKO enzyme for improved functional expression in E. coli. After a thorough bioinformatics analysis, several N-terminal truncated and modified KO enzymes were constructed (FIG. 9A). Constructs were created that incorporate SrKO and co-factor cytrochrome P450 reductase enzyme (SrCPR) as a fusion protein (“linker” constructs) or as a polycistronic modules (“operon” constructs) (FIG. 9B) in the pET45d expression vector. The production and relative solubility of the protein in these constructs in E. coli was assessed using SDS-PAGE analysis (FIG. 9C).

These constructs were then transferred into our production vector p5Trc to test the in vivo functional activity of the pathway. These constructs were transformed into kaurene producing strains 3, 9 and 11 (Table 2) to test the conversion of kaurene to kaurenoic acid (Table 3). The designed chimeric enzymes were functionally active, but the incomplete reactivity of the enzymes resulted in the production of kaurenol and kaurenal. Among all various N-terminal truncated KO constructs, the 39AA truncation of KO was more functionally active compared to 4 and 20 amino acid truncated constructs. Additionally, the SrKO and SrCPR expressed as operons showed similar activity as fusion enzyme constructed from SrKO and SrCPR. Subsequent to this initial work, we further optimized the SrKO enzyme as part of a three-gene KAH-KO-CPR module, see below, and in this construct the optimal SrKO construct has 20 amino acid residues truncated from the N-terminus, resulting in complete conversion of kaurene to kaurenoic acid (see below).

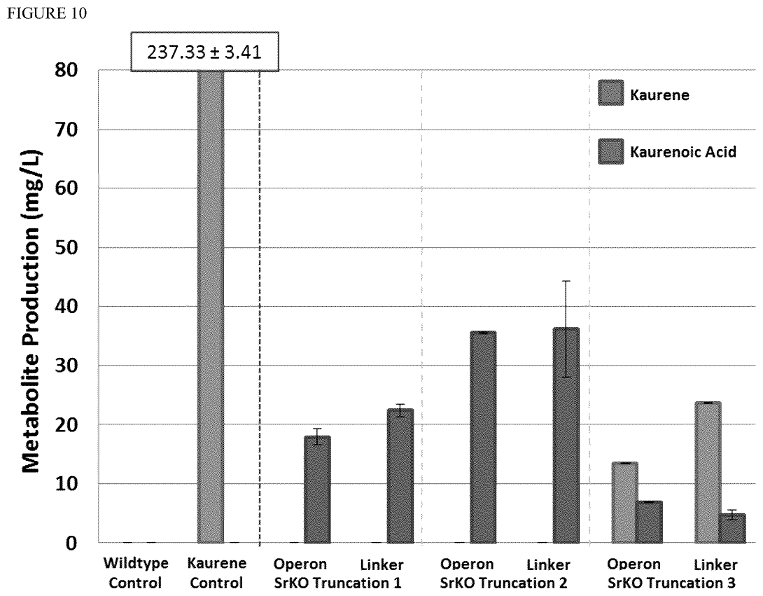

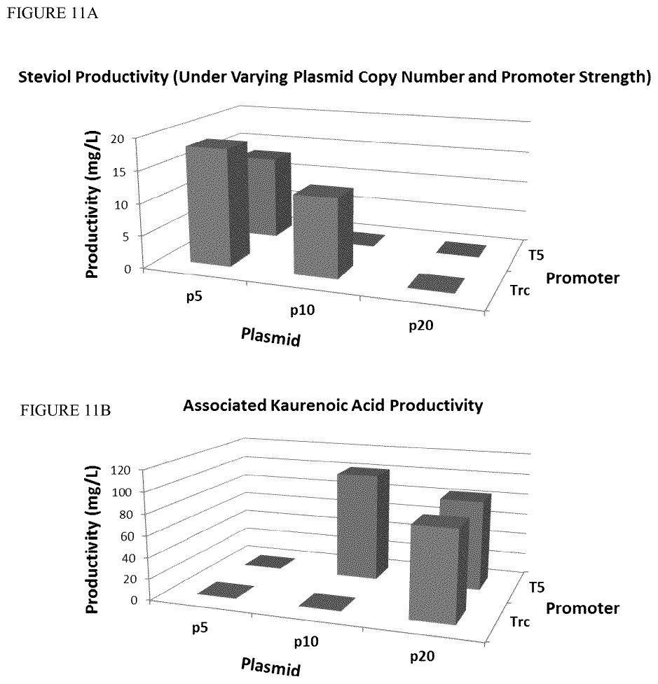

In the initial screening of the above SrKO strains, it was found that the availability of substrate pool for subsequent conversion to kaurenoic acid is important. When a high substrate (kaurene) pool was available (strain 9) the oxidation pathway converted kaurene to ˜60 mg/L of oxygenated kaurene compounds. Previous in vitro studies on the enzymatic activity of the Arabidopsis thaliana KO enzyme demonstrated that the enzyme produces the alcohol, diol. and aldehyde derivatives of kaurene (12). Similar product diversity is seen with taxol P450 enzymes. however, in that work rebalancing of the entire pathway changed the product profile and produced the hydroxylated taxanes exclusively. Therefore, strain engineering studies were initiated to rebalance the KO P450 module and upstream modules. In order to rebalance the modules. the KO pathway was transferred into the high kaurene-producing chromosomally-integrated strain (Strain 47) and tested for activity and productivity (FIG. 10). Orthologous KO enzymes from different organisms were also designed, synthesized and tested (Table 1) to identify the best variant enzymes. Multivariate pathway optimization was performed, as with the kaurene pathway. to identify the best variant enzyme and their non-linear product profiles and product distributions under varying flux balances (FIG. 11A and FIG. 11B). SrKO activity was subsequently improved by designing and testing a collection of point mutants in the wild-type background (Table 4).

Upon successful production of kaurenoic acid. the final enzymatic step in the biosynthetic pathway was incorporated and tested. hydroxylation at the C13 carbon of kaurenoic acid by the enzyme KAH to yield steviol (FIG. 1 and FIG. 2). Studies on the polycistronic expression of SrKO and SrCPR proved that this enzymes can be expressed as independent components and remain functionally active. It was determined whether both KO and KAH could be functionally active with a single SrCPR enzyme. In order to limit the number of plasmids and balance the expression of KO and KAH, a single copy SrCPR was chromosomally integrated into a kaurene engineered strain. The KO and KAH was constructed as polycistronic expression under p5Trc plasmid. Detectable levels of steviol were detected in GC-MS analysis of all strains (Table 5) after four days fermentation and extraction with ethyl acetate.

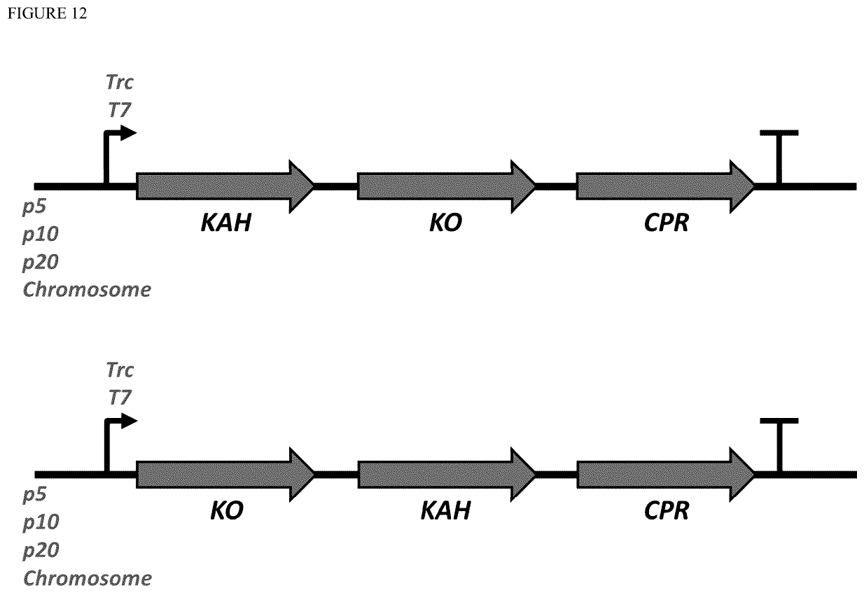

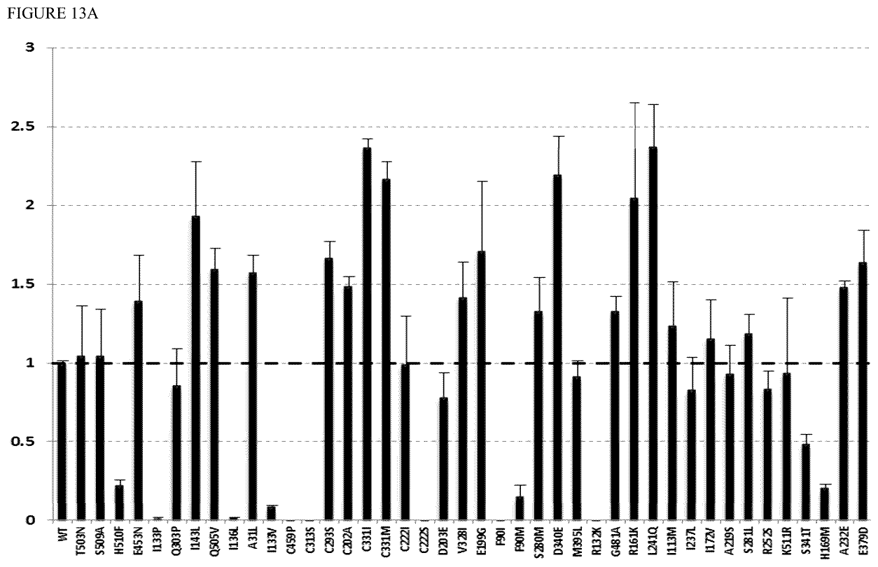

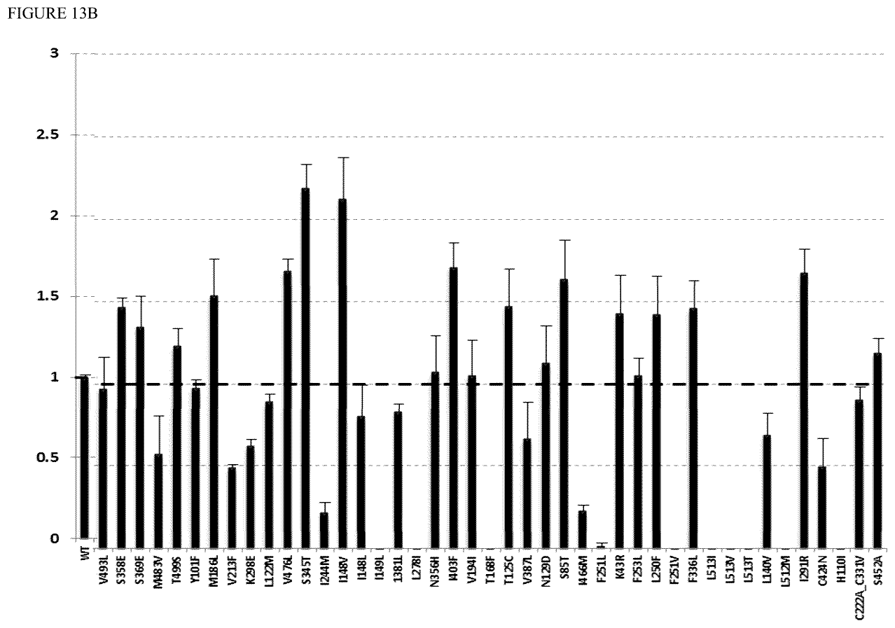

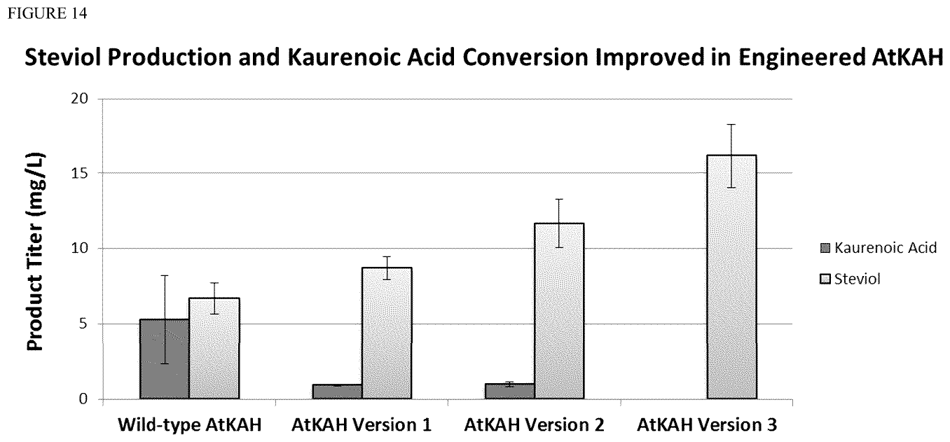

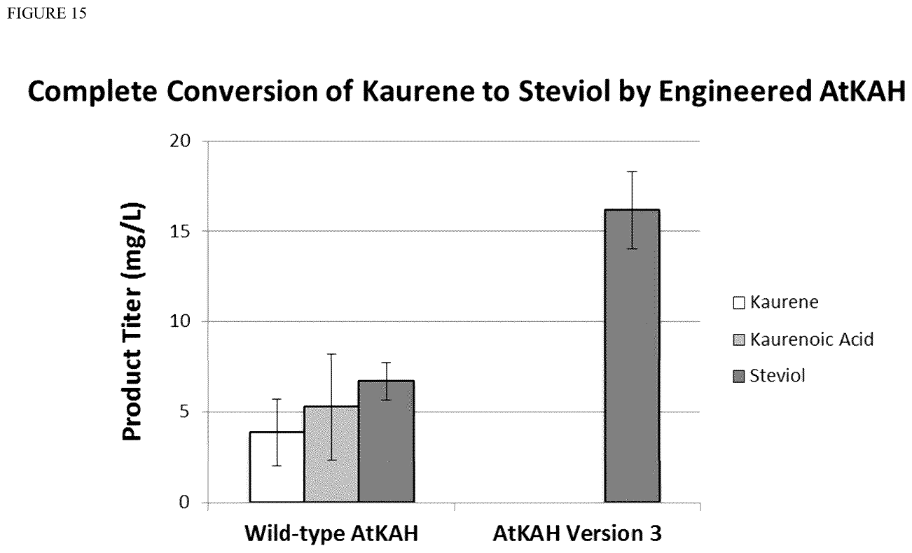

With this promising initial result in hand. the optimal enzymes for assembly into a biosynthetic pathway in E. coli were identified by in vivo expression of different engineered versions of both a KO and KAH candidate in a polycistronic operon with a CPR co-factor enzyme (FIG. 12). The AtKAH enzyme was further enhanced with a campaign of point mutations. A rational approach was used to design a collection of single point mutations in the AtKAH sequence, aimed at increasing stability, solubility, or activity of the wild-type enzyme for improved conversion of kaurenoic acid to steviol. The point mutations and corresponding fold-change improvements over wild-type AtKAH are summarized in Table 6, and are visualized in FIG. 13A and FIG. 13B. Some of these point mutations were then recombined in the AtKAH enzyme, and a recombinant enzyme was identified that was capable of complete conversion of kaurenoic acid to steviol (FIG. 14). When expressed in an operon with optimal SrKO and SrCPR, complete conversion of kaurene to steviol was demonstrated (FIG. 15). further highlighting the importance of careful balancing of pathway components.

(C) Engineering Small Molecule Glycosylation in E. coli

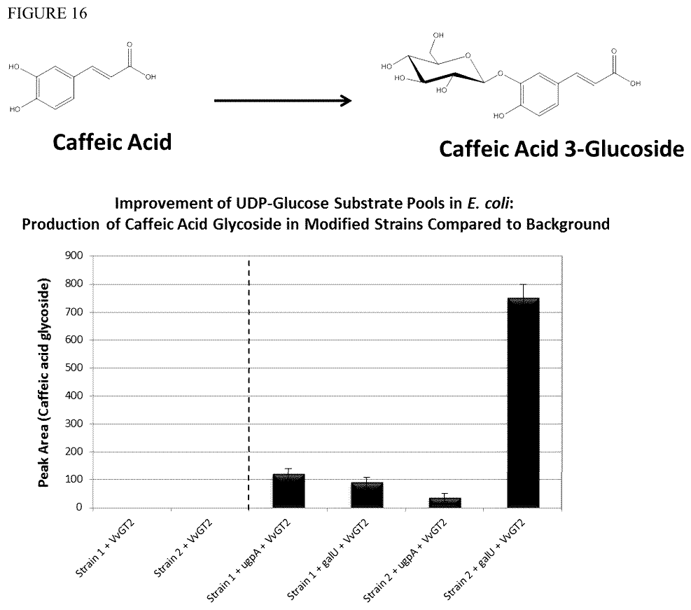

To support multiple glycosylation of steviol to rebaudioside M and all intermediate steviol glycosides, a series of modifications were introduced to the background E. coli strain intended to increase the amount of UDP-glucose available. A series of gene knock-outs and gene insertions were made aimed at increasing carbon flux to UDP-glucose and decreasing flux in pathways away from UDP-glucose not in keeping with small molecule glycosylation (i.e., glycogen synthesis and carbon storage). The design enables the import of sucrose into the cell and its splitting into fructose and glucose via the activity of sucrose phosphorylase. A subsequent series of knock-outs have altered primary metabolism so as to force biomass to be synthesized using only fructose as carbon source. leaving glucose to be funneled exclusively towards UDP-glucose biosynthesis when the cells are grown using sucrose as a carbon source. However, the cells are still capable of growth and improved UDP-glucose availability when grown on either glycerol or glucose as the carbon source. The specific modifications applied to the E. coli strain to enact this strategy are listed in Table 7. These modifications were tested to determine whether they enabled enhanced glycosylation of small molecules, and demonstrated that they indeed do by showing enhanced in vivo glycosylation of caffeic acid (supplemented in the media) by the engineered strains (FIG. 16).

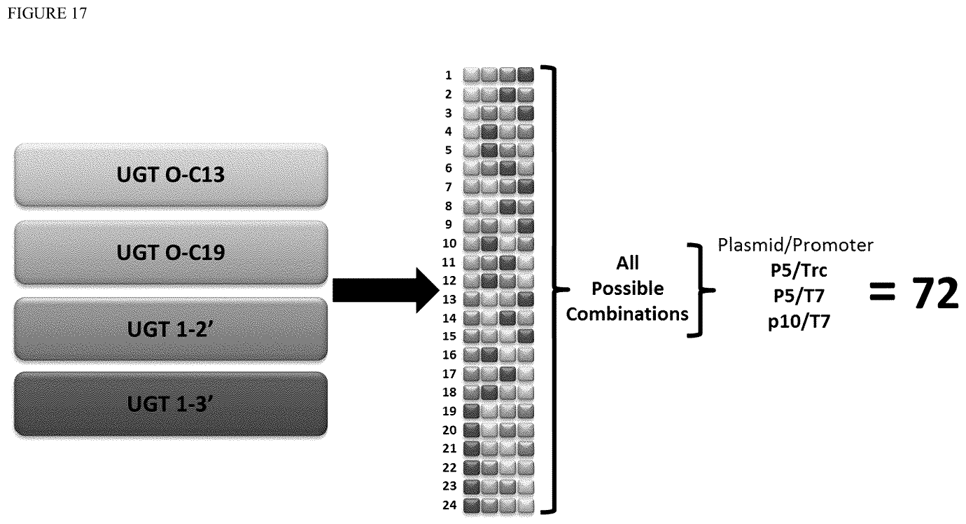

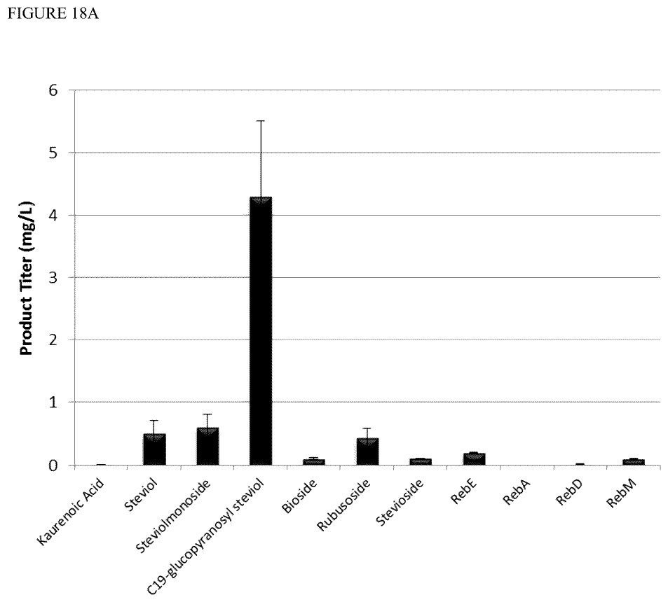

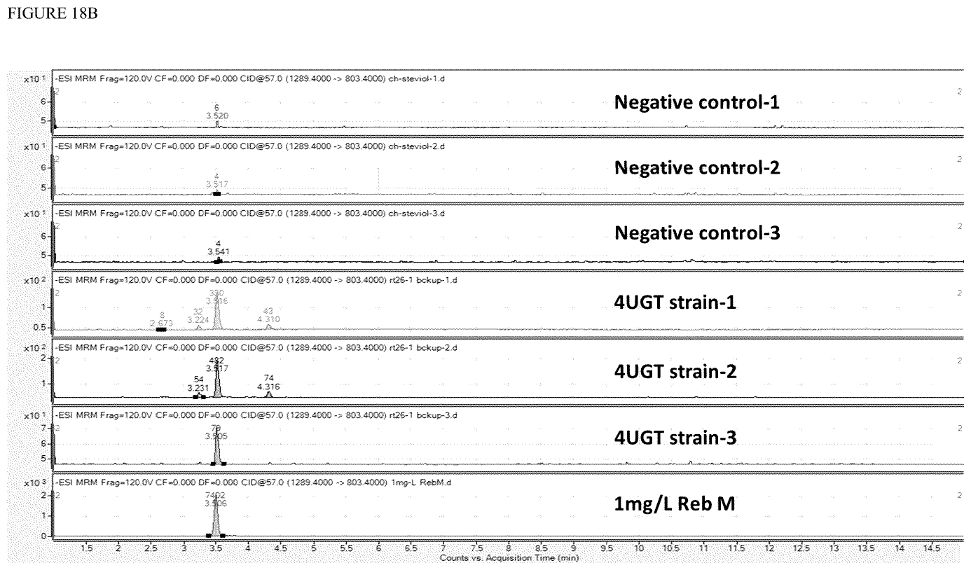

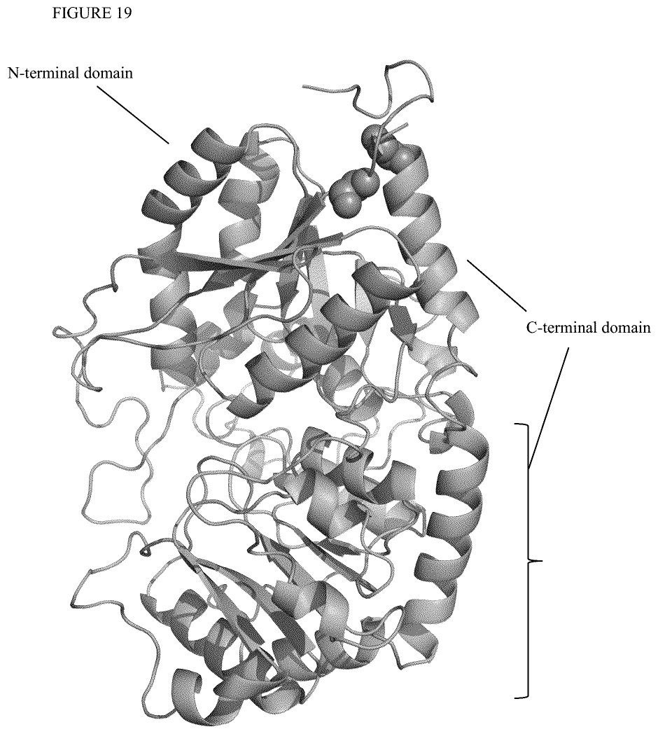

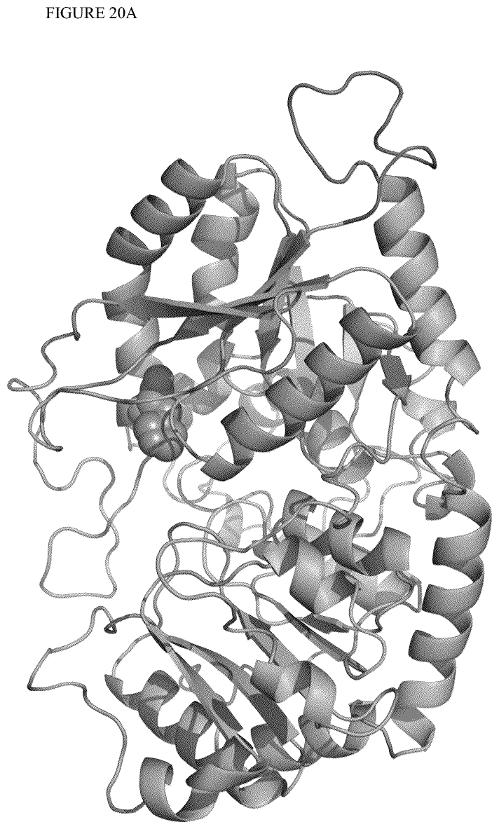

Having constructed the steviol core molecule, the core was glycosylated with UDP-glucose using an assembly of four UGTs, each capable of different glycosylation chemistries (Tables 8 and 9). These chemistries include (1)O-glycosylation at C13 of steviol. (2)O-glycosylation at C19 of steviol. (3) 1-2″-glycosylation at either the C13 or C19 O-glucose, and (4) 1-3-glycosylation at either the C13 or C19 O-glucose (see FIG. 1 for an example incorporating all the above chemistries). Once one or both of the C13 and/or C19 oxygens are glycosylated. further glycosylations can be added to the O-glucose via 1-2′ or 1-3′ additions. As described below. these UGTs have been fundamentally modified by the shuffling of their domains, and further enhanced by point mutations aimed and enhancing flux through to the desired end product of RebM. The MMME approach was applied to rapidly combine the four UGTs in all possible combinations and screen the resulting constructs in vivo in a steviol-producing strain background (FIG. 17). The improved UGTs assembled in the optimum polycistronic configuration were combined with the four other modules in a single E. coli strain. We cultured the strain and demonstrated in vivo production of steviol glycosides leading to rebaudioside M (FIG. 18A and FIG. 18B). The strain is capable of producing 5.7 mg/L of total steviol glycosides, which includes 100 μg/L of RebM. By generating all possible constructs and expressing them either in plasmids or integrated into the chromosome, under a variety of promoter strengths, steviol glycoside product profiles were obtained with RebD: RebM ratios ranging from 1:1 all the way to 0):1 (no RebD remaining).

To our knowledge, this is the first time two cytochrome P450 monooxygenases have been functionally expressed in E. coli for the production of a bifuncational oxygenated terpenoid molecule such as steviol. Additionally, a single CPR enzyme acted as co-factor for both P450 enzymes (KO and KAH) for converting kaurene to steviol. This is another significant leap in the engineering of P450 mediated oxidation chemistry in E. coli system. Moreover, to our knowledge, this is the first time four UGTs have been combined in a single E. coli strain and demonstrated to be capable of performing six sequential glycosylations of a terpenoid core molecule to produce rebaudioside M. let alone the intermediate steviol glycosides. This is a significant leap forward in the engineering of UGTs and the establishment of a platform for sustainable production of rare steviol glycosides.

### Example 2: Construction of Circular Permutants of Glycosyltransferase Enzymes

Natural selection acting on an enzyme tends to select for sufficient stability and activity for the biological function. This process sends an enzyme down a specific evolutionary path that may make it not readily compatible with the stability and activity gains needed for industrial applications. As an example, enzymes specialized for a specific substrate tend to be more challenging to engineer for new substrates than enzymes that have not been specialized. Thus, ‘shaking up’ an enzyme by swapping domain connections might create an enzyme with the same protein fold. yet with novel folding and folded interactions that would make it newly-amenable to selection and evolution. In other words, we might be able to ‘jump’ a protein fold to another point in evolutionary space simply by shuffling the sequence, moving the enzyme away from its original evolutionary path without introducing any amino acid mutations.

UGTs (UDP-glucose glycosyltransferases) have two domains, a more variable N-terminal substrate binding (sugar acceptor) domain and a more conserved C-terminal UDP-glucose binding (sugar donor) domain. The N-terminal domain is mostly determinant of substrate specificity for the enzyme, but some specificity is controlled by the C-terminal domain. Each of these domains makes up roughly half of the protein. Given this two-domain structure, we hypothesized that cutting the protein in half to create new N- and C-termini and attaching the originals together (e.g., circular ‘permutization’) would shuffle the enzyme and create new opportunities for engineering improved activity (since the resulting enzyme would not be the result of selective pressure).

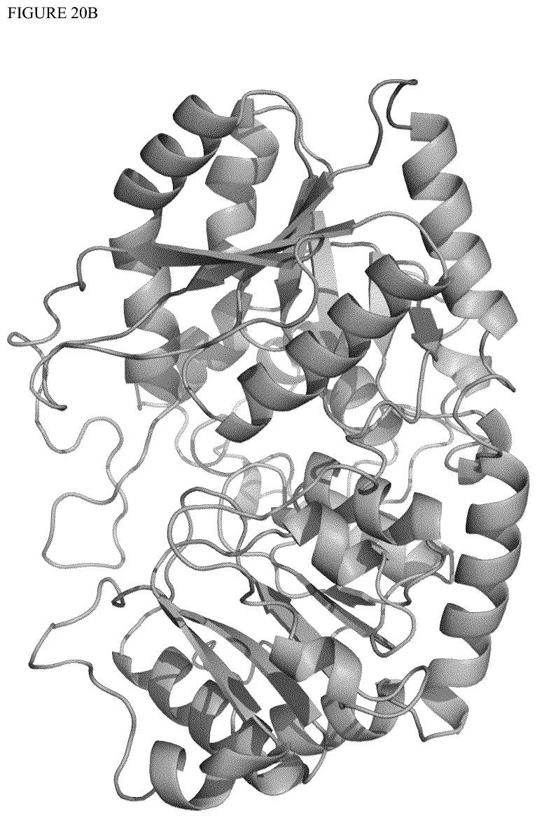

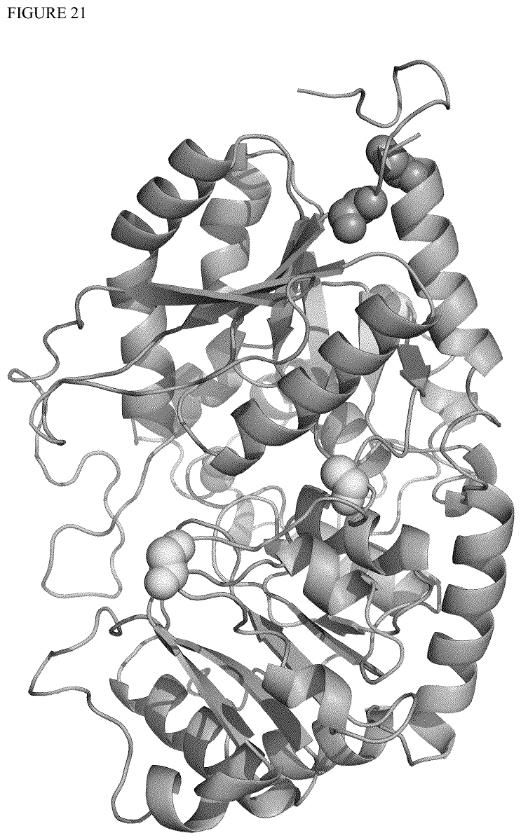

As a description of general procedure, designing a shuffled enzyme involves the following steps: (i) create a homology model to a known UGT with desired glycosylation activity (FIG. 19): (ii) using the homology model, estimate distance between N- and C-terminal residues: (iii) design linkers of various lengths to connect the existing N- and C-termini: (iv) select positions in the enzyme to become the new N- and C-termini: (v) synthesize the resulting sequences: (vi) express in vivo and in vitro and identify any shuffled enzymes that retain parent activity: (vii) modify designs via rational engineering, informed by a new homology model of the shuffled enzyme; and (viii) repeat step vii until desired activity improvement is achieved. When creating linkers (step iii), identify the N- and C-termini residues closest to ends, but predicted to be directly interacting with the rest of the protein based on the structure in the homology model (FIG. 20A, FIG. 20B, and FIG. 20C). When choosing cut sites for new N- and C-termini (step iv) (FIG. 21), choose a loop region between secondary structure elements, which maintains domain structure, is close to the middle of the sequence as possible, and which is solvent exposed and away from the active site.

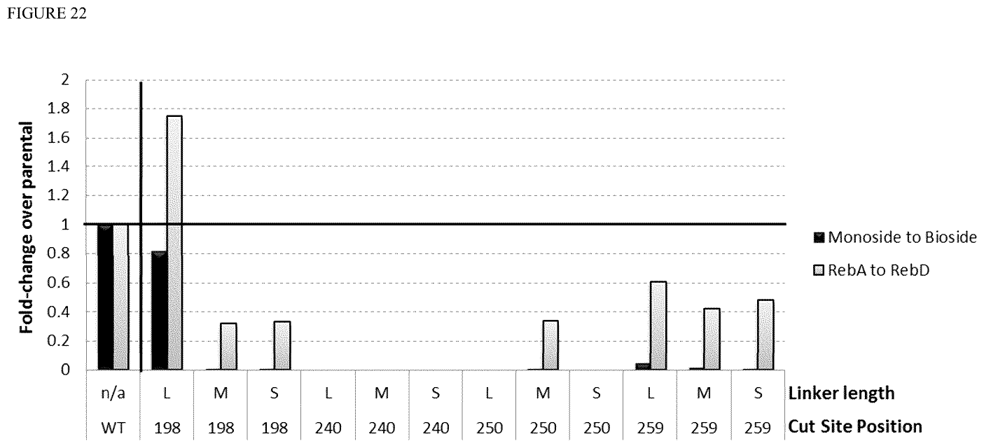

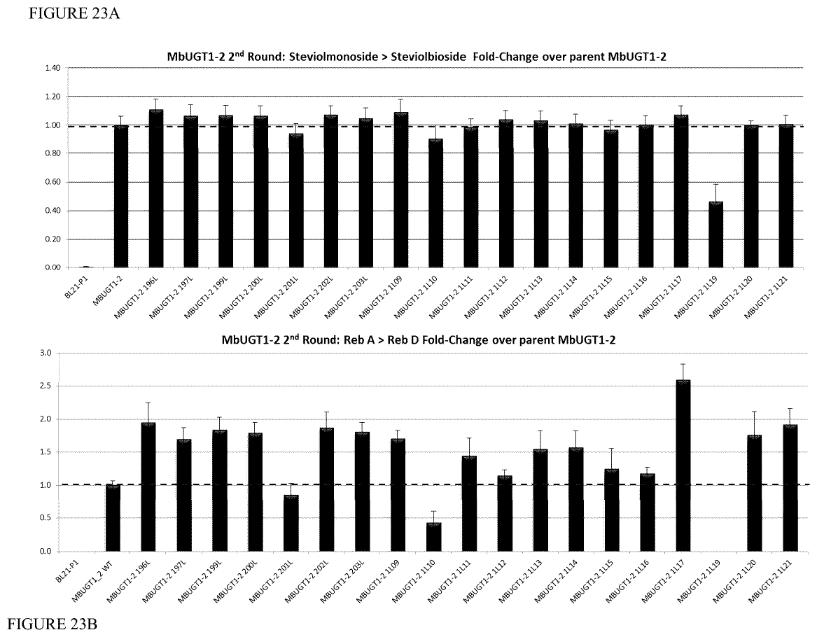

Circular permutants were designed, synthesized, and incorporated for each of the C13. C19, 1-2′ and 1-3′ glycosylating activities (parent enzymes are SrUGT85C2. SrUGT74G1. OsUGT1-2 (Q0DPB7_ORYSJ) (28), and SrUGT76G1. respectively). All four activities resulted in working circular permutants. As an example, data demonstrating the first round of circular permutant engineering resulting in the novel enzyme MbUGT1-2, showing equivalent activity with the parent enzyme OsUGT1-2 (FIG. 22). A subsequent round of refinement to the shuffled enzyme has generated enzymes with enhanced activities compared to the original parent sequence, demonstrating the potential for this shuffling approach to generate improved enzymes (FIG. 23A and FIG. 23B). These refinements focus on finer-scale modifications to the position and specific residues forming the novel N and C-termini of the shuffled enzyme, as well as refining the length and amino acid sequence of the linker connecting the parental N and C-termini.

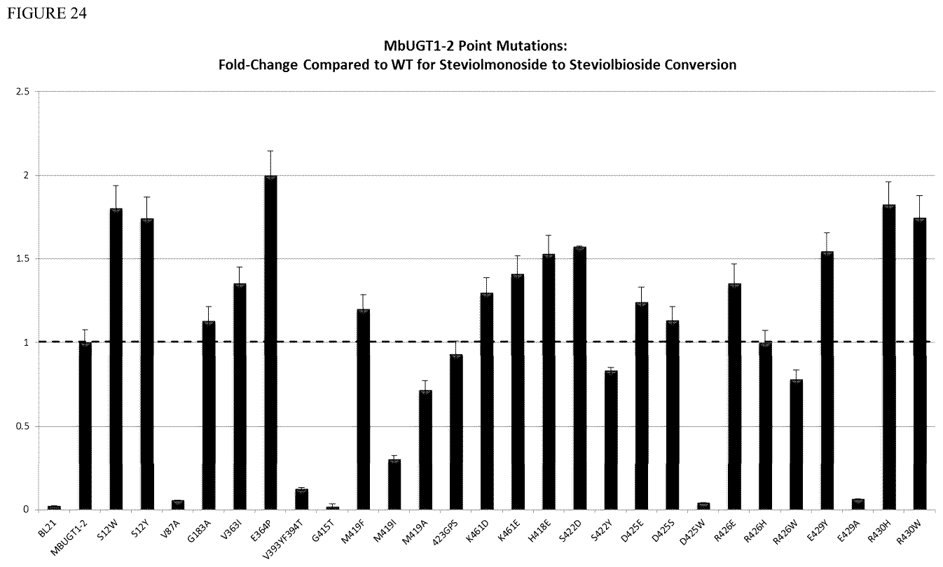

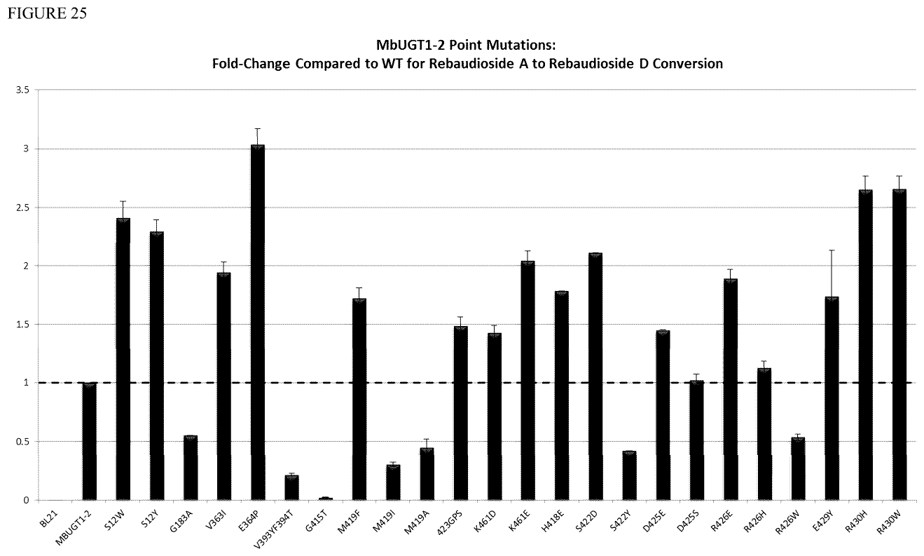

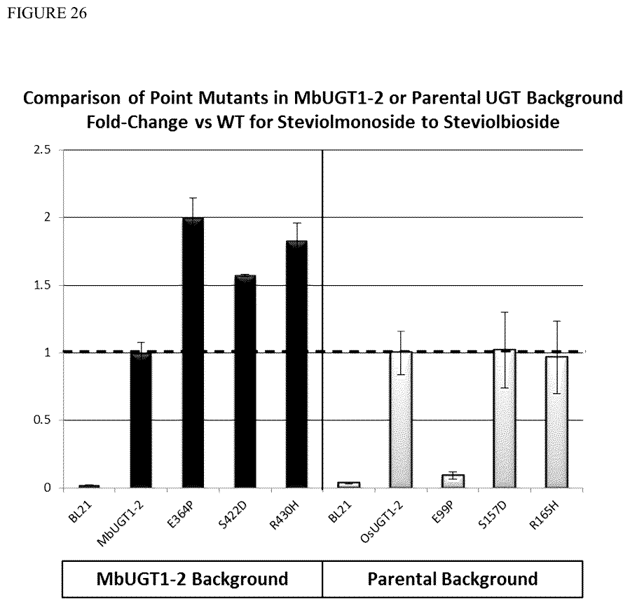

A series of point mutations to the MbUGT1-2 enzyme has resulted in further improvement to this novel sequence (FIG. 24 and FIG. 25), confirming that the process has created opportunities for significant improvement by shuffling the enzyme sequence. However, when these point mutations conferring improved activity in MbUGT1-2 were tested in the parent OsUGT1-2 background, they were found to be either neutral or deleterious to activity (FIG. 26). The lack of transferability of point mutations between the novel enzyme and the parent sequence confirms that circular permutization is a valuable and general approach for creating opportunities for UGT enzyme improvement.

### Example 3: Chimeric Fusions of Distinct Glycosyltransferases

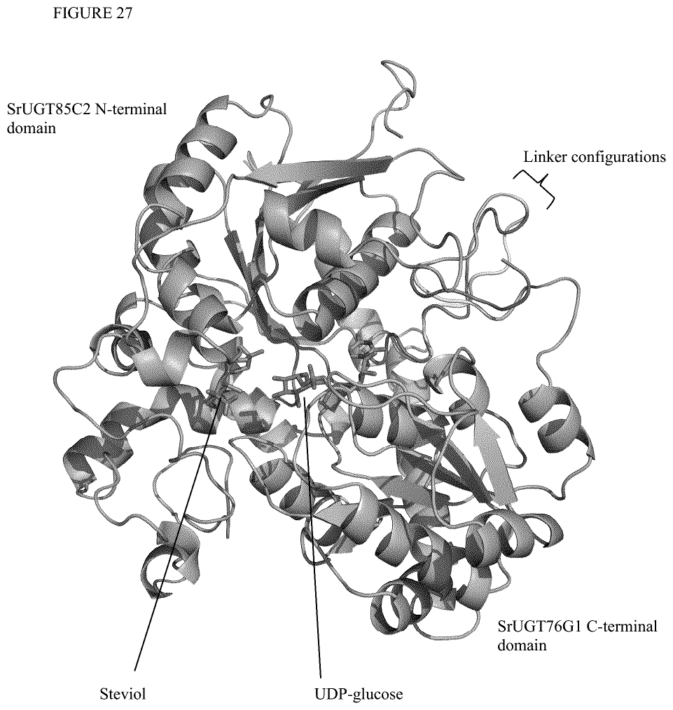

In a similar vein to the shuffling of enzymes engendered by circular permutization, an alternate method to shuffle glycosyltransferase enzymes was created by swapping the N-terminal small molecule sugar acceptor binding domain or the C-terminal sugar-donor binding domain between known UGTs. For example, a chimeric enzyme composed of the N-terminal domain of SrUGT85C2 and the C-terminal domain of SrUGT76G1 was created (FIG. 27).

The rationale behind this approach rests on the concept of shuffling the domains of a UGT enzyme, only this time we add the nuance of shuffling domains between UGTs. The intent is to generate a non-optimized enzyme with a novel sequence, capable of further evolution away from the point in the energy landscape occupied by the parent enzyme, and towards a new optimum enzyme configuration in the production strain. Again, given that this enzyme is not a result of natural selection per se, the shuffled enzyme resulting from this chimeric approach should have increased evolutionary potential/greater potential to benefit positively from point mutations (i.e., with increased activity). Moreover, this approach can be used to generate chimeric protein with enhanced folding and/or stability.

In brief, this approach employs four broad steps: (1) identify two candidate UGTs: (2) select crossover positions for making a chimera between the two UGTs (i.e., select the point at which to join the two sequences): (3) mutate the C-terminal domain (the nucleotide-carbohydrate binding domain, e.g. UDP-glucose binding domain) to improve interaction with the small molecule substrate or the N-terminal small-molecule binding domain, based on structural considerations: (4) create and test chimeric constructs for activity. This approach is generalizable and applicable for improving the functional performance of potentially any UGT. Given the conserved domain structure of UGTs, domains from any two UGTs could be recombined.

### Example 4: Modifying Glycosyltransferase Enzymes for Improved Activity and Biosynthesis of Rare Glycosides

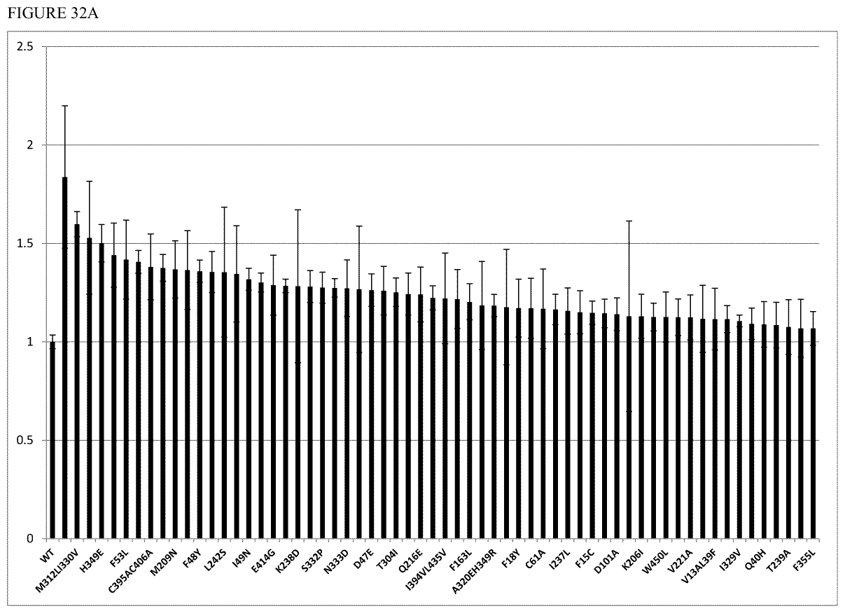

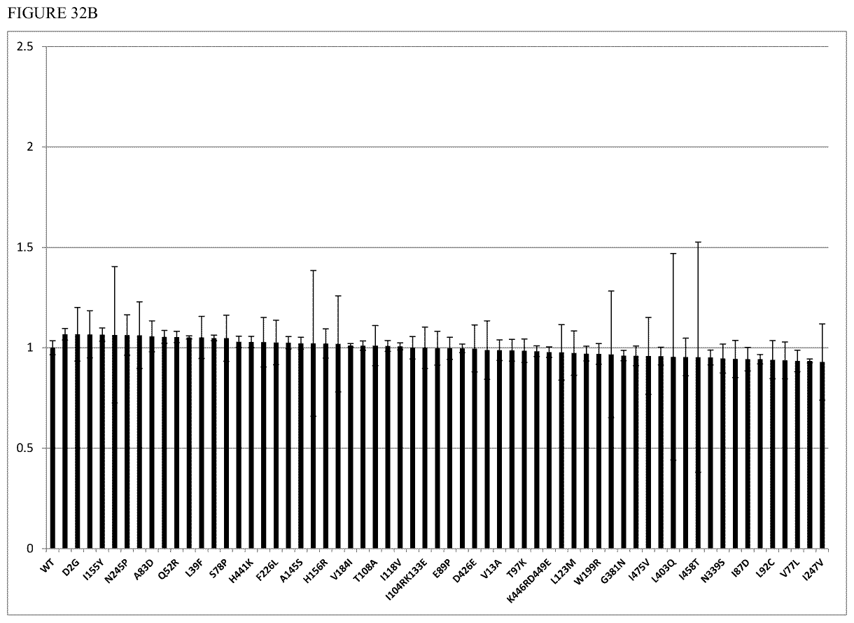

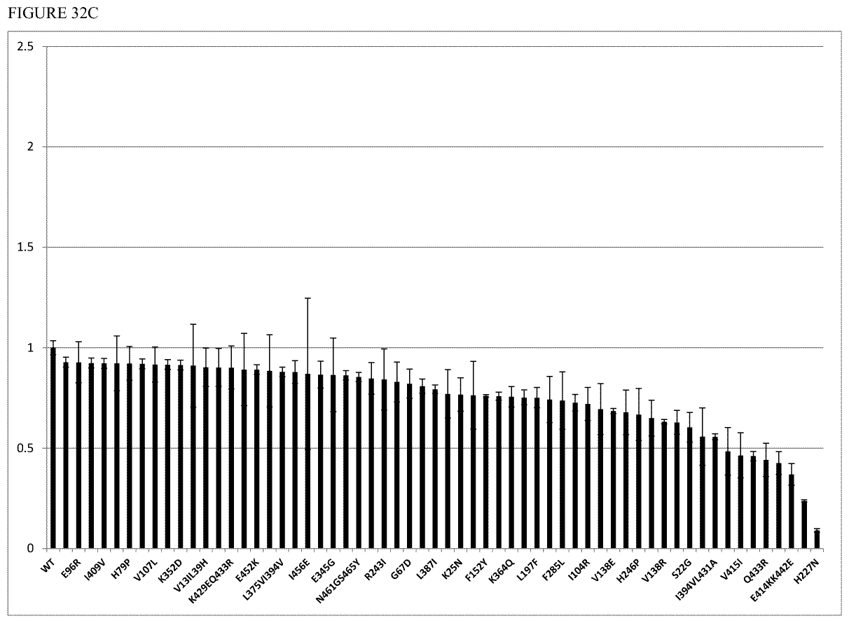

Although only around 20 steviol glycosides occur in sufficient quantities to have been characterized from stevia plants, there are several more possible steviol glycosides with different glycosylation patterns that can be created biosynthetically. Table 10 and FIG. 29, FIG. 30, FIG. 31 and FIG. 32A, FIG. 32B, and FIG. 32C summarize the known and potential steviol glycosides described in this section, which are abbreviated with the symbol SG #. Some of these glycosides exist in nature, and others are biosynthetically possible using UGTs that catalyze the four glycosylation chemistries described herein (i.e., C13-O-glycosylation, C19-O-glycosylation, 1,2′-glycosylation, and 1,3′-glycosylation).

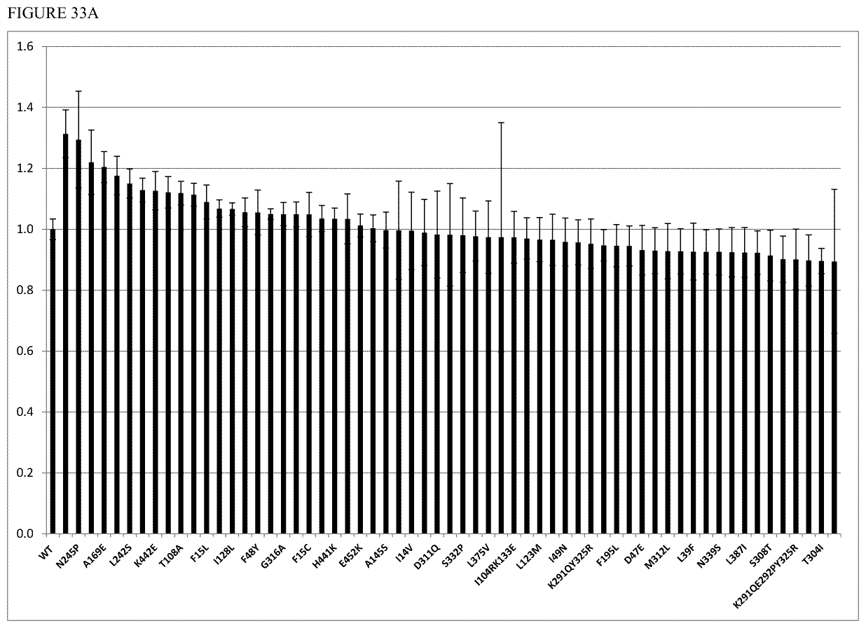

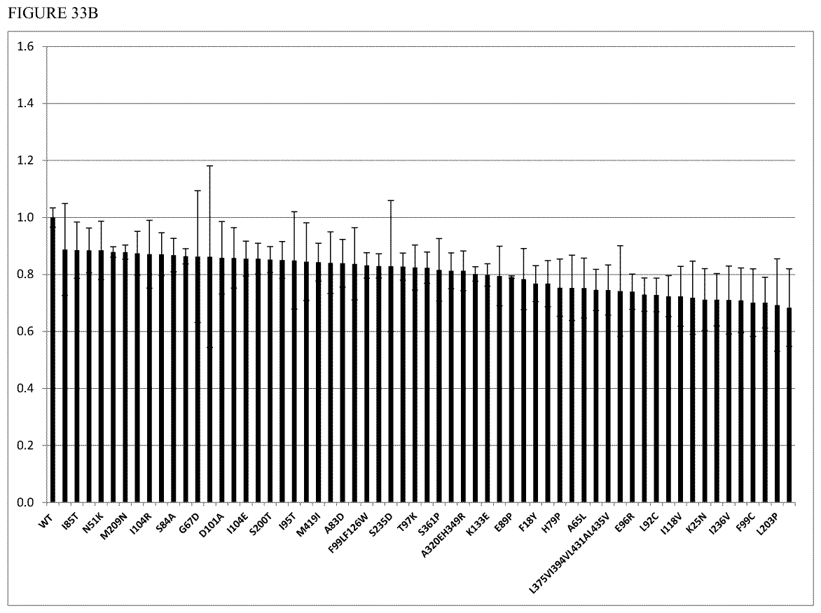

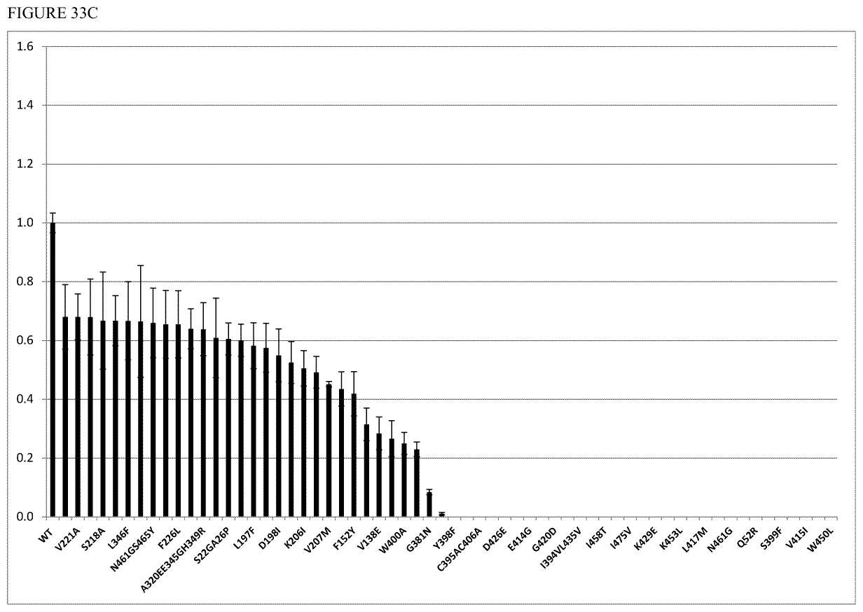

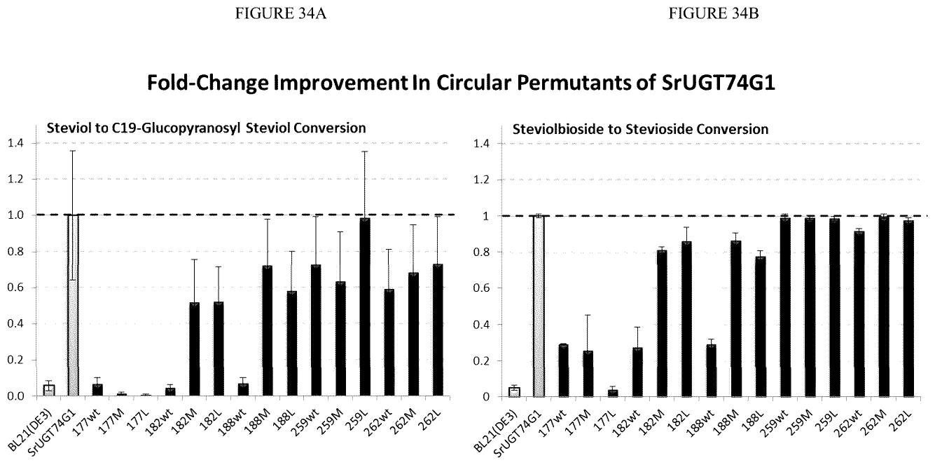

A rational design approach was used to design a collection of single, double, and triple point mutations in the SrUGT85C2 sequence (possessing C13-O-glycosylating activity), aimed at increasing stability, solubility, or activity of the wild-type enzyme for improved conversion of steviol to steviolmonoside (SG1), and/or C19-glucopyranosyl steviol (SG2) to rubusoside (SG5), and/or SG4 to SG10, and/or SG7 to SG11, and/or SG13 to SG17, and/or SG19 to SG29, and/or SG23 to SG31. The point mutations and corresponding fold-change improvements over wild-type SrUGT85C2 are summarized in Table 11, and are visualized in FIG. 32A, FIG. 32B, FIG. 32C, and FIG. 33A, FIG. 33B, and FIG. 33C.

A rational design approach was used to design a collection of single, double, and triple point mutations in the SrUGT74G1 sequence (possessing C19-O-glycosylating activity), aimed at increasing stability, solubility, or activity of the wild-type enzyme for improved conversion of steviol to C19-glucopyranosyl steviol (SG2), and/or steviolmonoside (SG1) to rubusoside (SG5), and/or steviolbioside (SG3) to stevioside (SG8), and/or SG6 to rebaudioside G (SG9), and/or rebaudioside B (SG12) to rebaudioside A (SG16), and/or SG18 to SG28, and/or SG22 to SG30.

A rational design approach was used to design a collection of single, double, and triple point mutations in the MbUGT1-2 sequence (possessing 1-2′ glycosylating activity), aimed at increasing stability, solubility, or activity of the wild-type enzyme for improved conversion of steviolmonoside (SG1) to steviolbioside (SG3), and/or C19-glucopyranosyl steviol (SG2) to SG4, and/or rubusoside (SG5) to stevioside (SG8), and/or rubusoside (SG5) to SG10, and/or rubusoside (SG5) to rebaudioside E (SG14), and/or SG6 to rebaudioside B (SG12), and/or SG7 to SG13, and/or stevioside (SG8) to rebaudioside E (SG14), and/or SG10 to rebaudioside E (SG14), and/or rebaudioside G (SG9) to rebaudioside A (SG16), and/or rebaudioside G (SG9) to SG21, and/or SG1l to SG17, and/or SG1l to SG20, and/or rebaudioside B (SG12) to SG22, and/or SG13 to SG23, and/or SG15 to rebaudioside I (SG26). and/or SG15 to SG27, and/or rebaudioside A (SG16) to rebaudioside D (SG24), and/or rebaudioside A (SG16) to SG30, and/or SG17 to SG25, and/or SG17 to SG31, and/or SG20 to SG25, and/or SG21 to rebaudioside D (SG24), and/or rebaudioside D (SG24) to SG37, and/or SG25 to SG39, and/or rebaudioside I (SG26) to rebaudioside M (SG32), and/or rebaudioside I (SG26) to SG38, and/or SG27 to rebaudioside M (SG32), and/or SG27 to SG40, and/or SG28 to SG33, and/or SG29 to SG35, and/or SG30 to SG37, and/or SG31 to SG39, and/or rebaudioside M (SG32) to SG43, and/or rebaudioside M (SG32) to SG44, and/or SG34 to SG41. and/or SG36 to SG42, and/or SG38 to SG43, and/or SG40 to SG44, and/or SG41 to SG47. and/or SG42 to SG48, and/or SG43 to SG46, and/or SG44 to SG46. The point mutations and corresponding fold-change improvements over wild-type MbUGT1-2 are summarized in Table 12, and representative reactions are shown in FIG. 24 and FIG. 25.

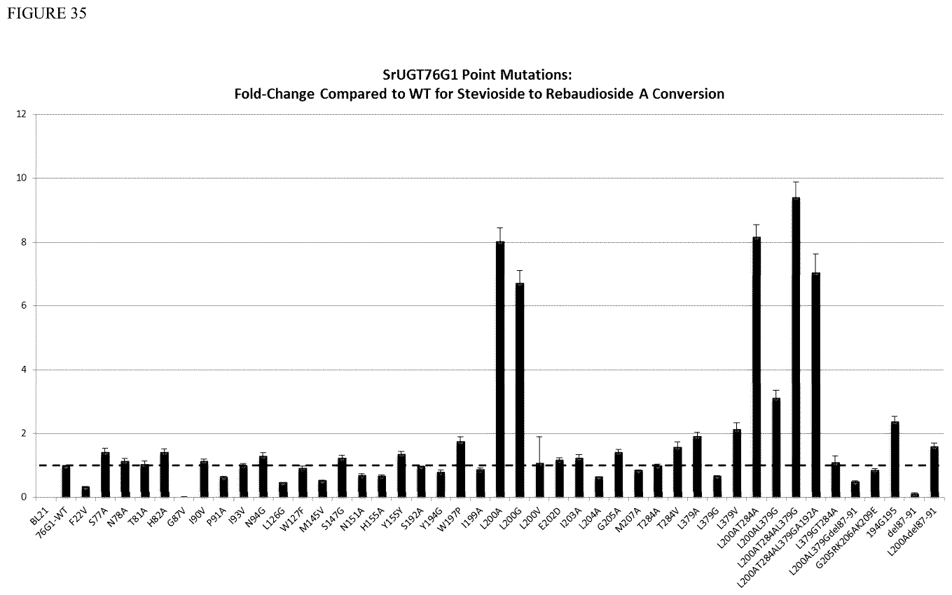

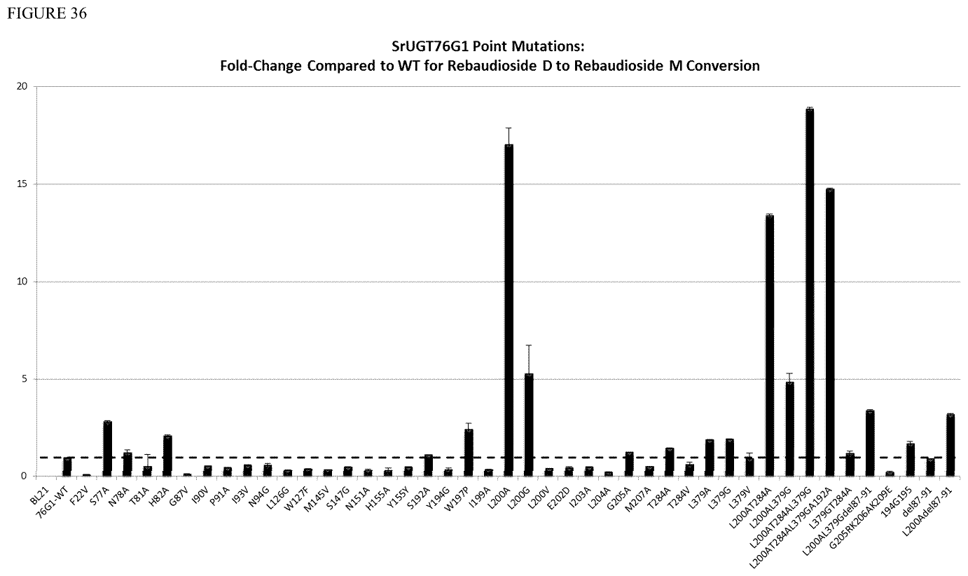

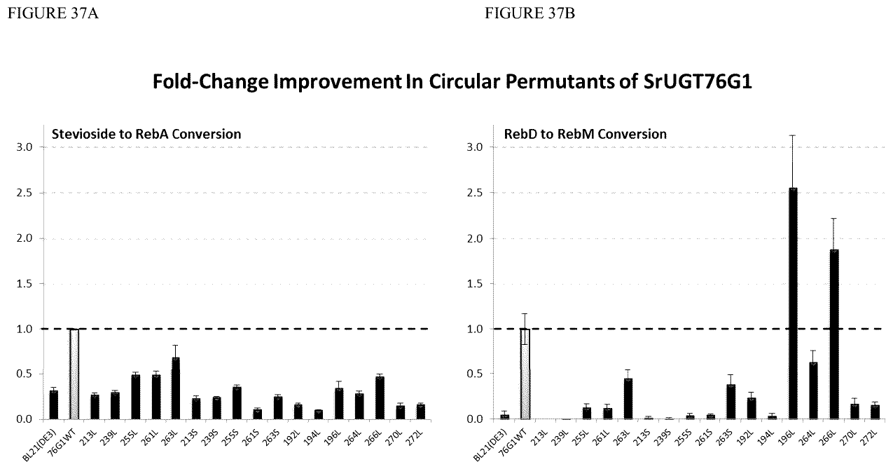

A rational design approach was used to design a collection of single, double. triple, or quadruple point mutations in the SrUGT76G1 sequence (possessing 1-3′ glycosylating activity), aimed at increasing stability, solubility, or activity of the wild-type enzyme for improved conversion of steviolmonoside (SG1) to SG6, and/or C19-glucopyranosyl steviol (SG2) to SG7, and/or steviolbioside (SG3) to rebaudioside B (SG12), and/or SG4 to SG13. and/or rubusoside (SG5) to rebaudioside G (SG9), and/or rubusoside (SG5) to SG11, and/or rubusoside (SG5) to SG15, and/or stevioside (SG8) to rebaudioside A (SG16), and/or stevioside (SG8) to SG20, and/or rebaudioside G (SG9) to SG15, and/or SG10 to SG17, and/or SG10 to SG21, and/or SG11 to SG15, and/or rebaudioside B (SG12) to SG18, and/or SG13 to SG19, and/or rebaudioside E (SG14) to rebaudioside D (SG24), and/or rebaudioside E (SG14) to SG25, and/or rebaudioside E (SG14) to rebaudioside M (SG32), and/or rebaudioside A (SG16) to rebaudioside I (SG26), and/or rebaudioside A (SG16) to SG28, and/or SG17 to SG27, and/or SG17 to SG29, and/or SG20 to rebaudioside I (SG26), and/or SG21 to SG27, and/or rebaudioside D (SG24) to rebaudioside M (SG32), and/or rebaudioside D (SG24) to SG33, and/or SG25 to rebaudioside M (SG32), and/or SG25 to SG35, and/or rebaudioside I (SG26) to SG34, and/or SG27 to SG36, and/or SG28 to SG34, and/or SG29 to SG36, and/or SG30 to SG38, and/or SG31 to SG40, and/or rebaudioside M (SG32) to SG41, and/or rebaudioside M (SG32) to SG42, and/or SG33 to SG41, and/or SG35 to SG42, and/or SG37 to SG43, and/or SG39 to SG44, and/or SG41 to SG45, and/or SG42 to SG45, and/or SG43 to SG48, and/or SG44 to SG47. The point mutations and corresponding fold-change improvements over wild-type SrUGT76G1 are summarized in Table 13, and representative reactions are shown in FIG. 35 and FIG. 36.

### Example 5: Improving Yield and Performance Above 22° C.

The performances of the enzymes in the kaurene module were determined to be suboptimal at temperatures above 22° C. A cluster of alternative enzymes were identified for the GGPPS (geranylgeranyl diphosphate) synthase enzyme and the bi-functional copalyl diphosphate (CPP)/kaurene synthase enzymes used in the previous examples. In particular, alternate enzymes from bacterial sources were considered, reasoning that these may function better in E. coli than plant and fungal enzymes. Enzymes from thermophilic bacteria were considered where possible. For the CPP synthase and kaurene synthase activities, genes from bacteria in the rhizosphere were identified, since they are often kaurene-producing due to their symbiotic lifestyle.

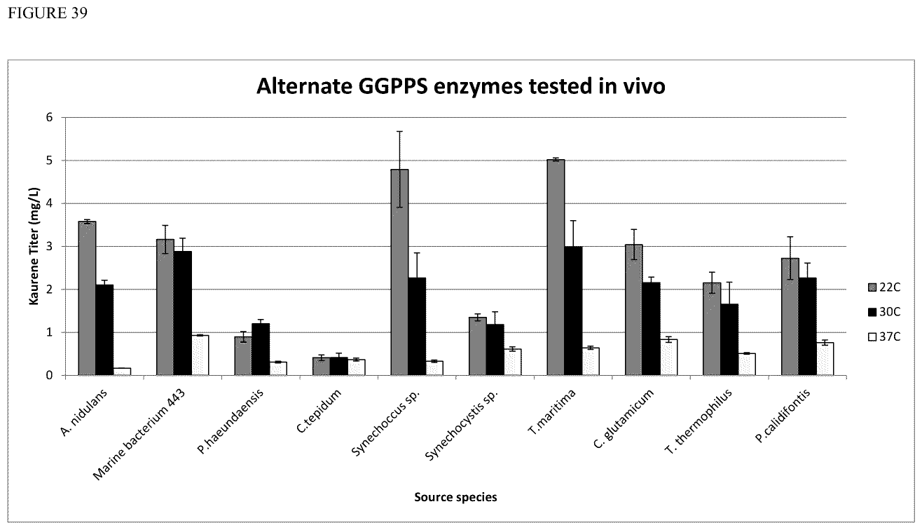

FIG. 39 shows the results for alternate GGPPS enzymes. Several enzymes show improved performance at higher temperatures, including Marine bacterium 443, Synechoccus sp., Thermotoga maritima. Cornybacterium glutamicum, and Pyrobaculum calidifontis.

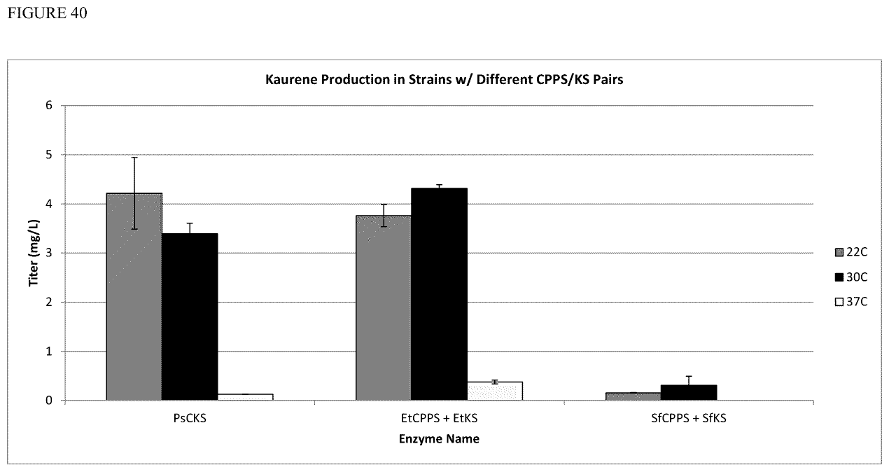

FIG. 40 shows the results for alternate CPPS and KS enzymes. Erwina tracheiphila (Et)CPPS and EtKS showed improved activity at higher temperatures.

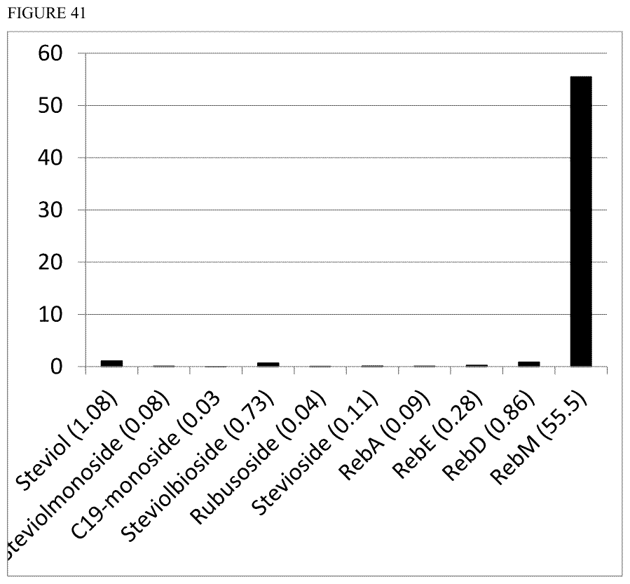

Production of various steviol glycosides (including Reb M) was tested at 22° C. in a select strain. The strain was E. coli K12 with a pBAC single-copy chromosome containing FAB46-MEP, T7-PsCKS-AnGGPPS, T7-AtKAH-SrKO-SrCPR, T7-MbUGT1,3-MbUGT1,2-MbUGTc13-MbUGTc19. As shown in FIG. 41, Reb M titer was 55.5 mg/L with a total steviol glycoside titer of 58.3 mg/L, which is equal to 94.4% Reb M. The Reb M:Reb D ratio was 64.5:1 (in grams).

| Statistic | Quantity |
|---|---|
| Titer, Total Steviol Glycosides (mg/L) | 58.3 mg/L |
| Titer, Rebaudioside M (mg/L) | 55.5 mg/L |
| % Reb M (of total glycosides) | 94.4% |
| Reb M:Reb D (g/g) | 64.5:1 |

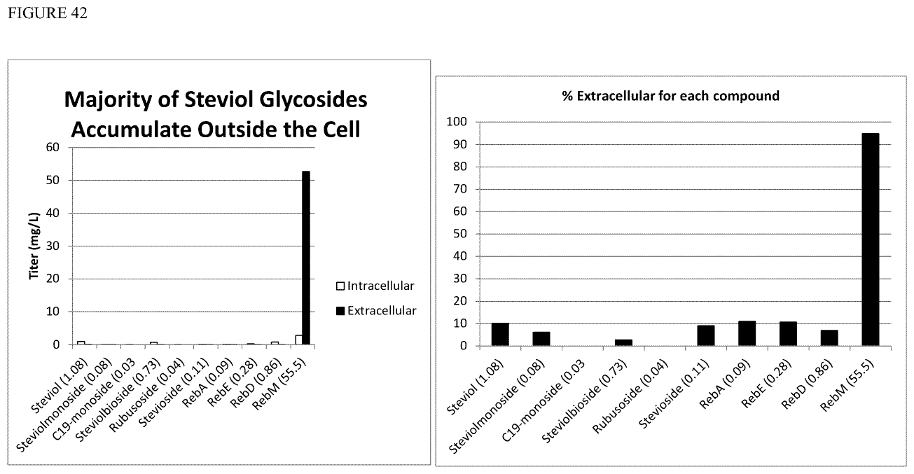

The intracellular accumulation of steviol glycosides was investigated. As shown in FIG. 42, the majority of the steviol glycosides are excreted from the cell. FIG. 42 shows the combined intracellular and extracellular material, as a percentage of product accumulating inside the cell versus outside. This was in contrast to initial studies having substantially less yield of steviol glycosides, which saw mostly intracellular accumulation. It's possible that the initial studies were of such low titer that accumulated product pools were insufficient for the active transport mechanisms required to pump the product out of the cells. Indeed, as the titer increased, a greater proportion of the product accumulated outside the cell, indicating that once above the threshold concentration for the putative pump activity, the rest of the products get moved out. These data are very promising from a strain engineering perspective and commercial production in E. coli, since if intermediate product pools are maintained below the Kb of the transporter, we can effectively push C-flux through to the end product without losing carbon to the outside (e.g., once a steviol glycoside intermediate is pumped out, it can no longer be further glycosylated to the desired product, such as RebM).

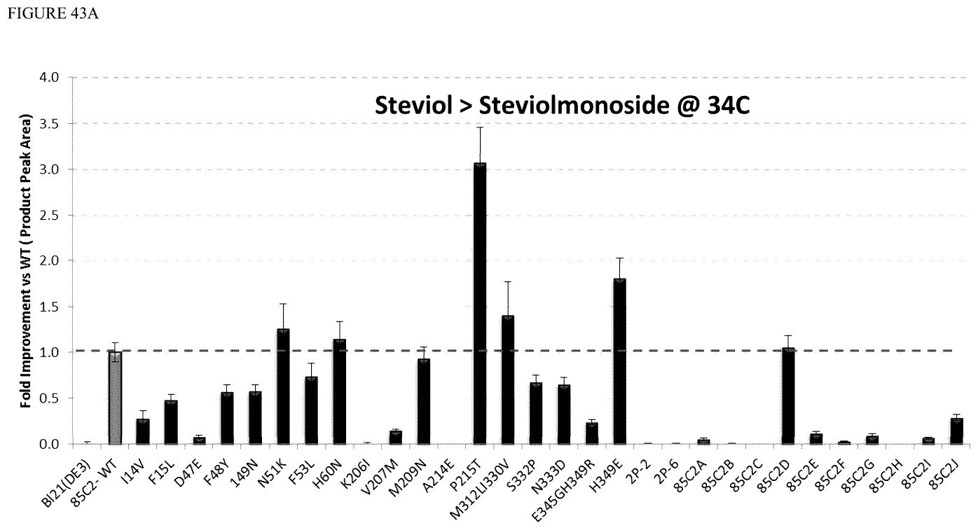

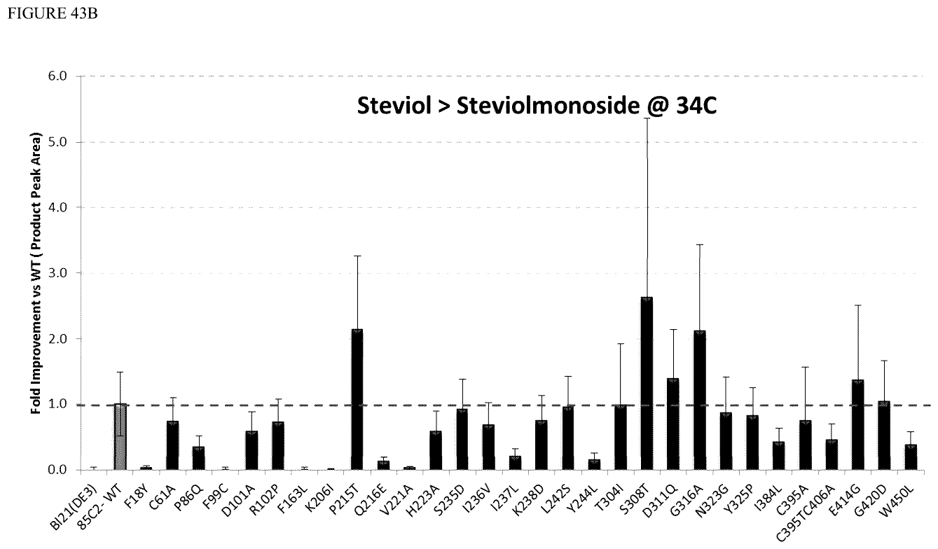

Point mutants of UGT85C2 were generated, and tested at 22, 30, and 34° ° C. FIG. 43A, and FIG. 43B show steviol monoside production at 34° C. FIG. 43C shows production of steviolmonoside with selected mutants at 22, 30, and 34° C. Several mutations showed higher production of steviolmonoside at 34° C., with P215T being the highest producing mutation.

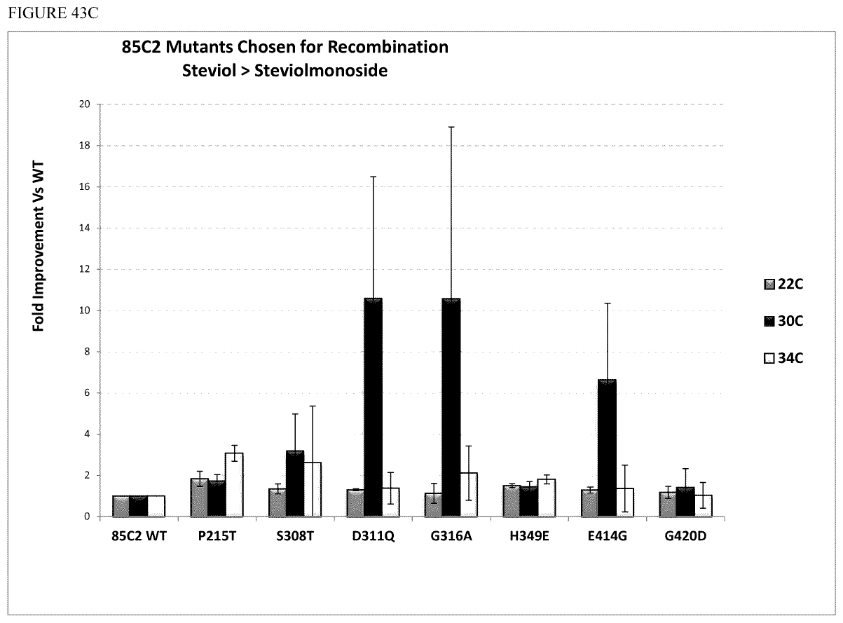

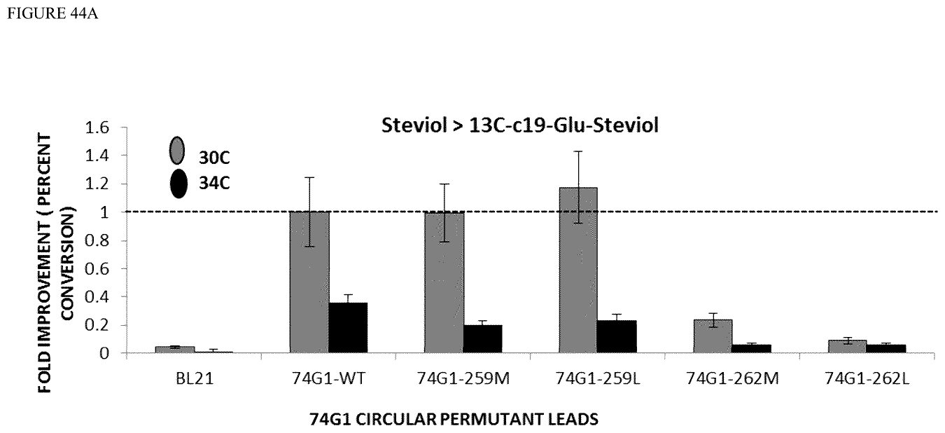

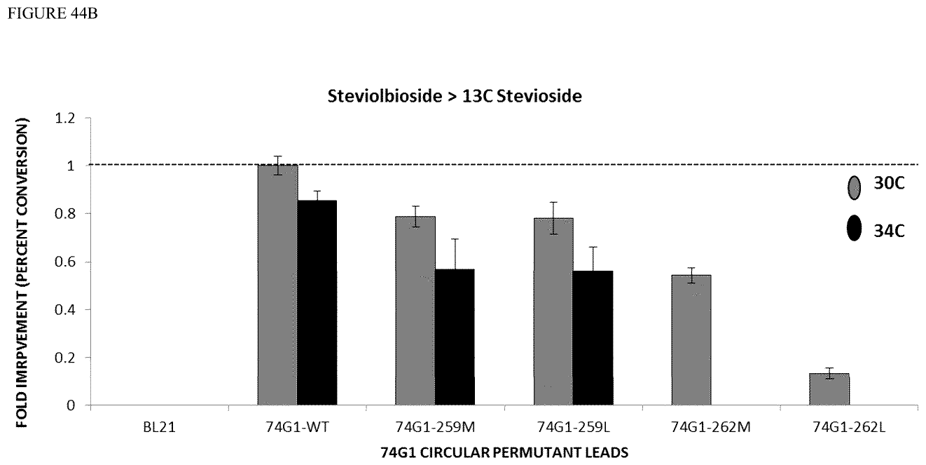

Circular permutants of 74G1 were also tested for activity at 30 and 34° C. FIG. 44A and FIG. 44B show conversion of steviol to 13C-c19-Glu-Steviol (FIG. 44A) and steviolbioside to 13C Stevioside (FIG. 44B).

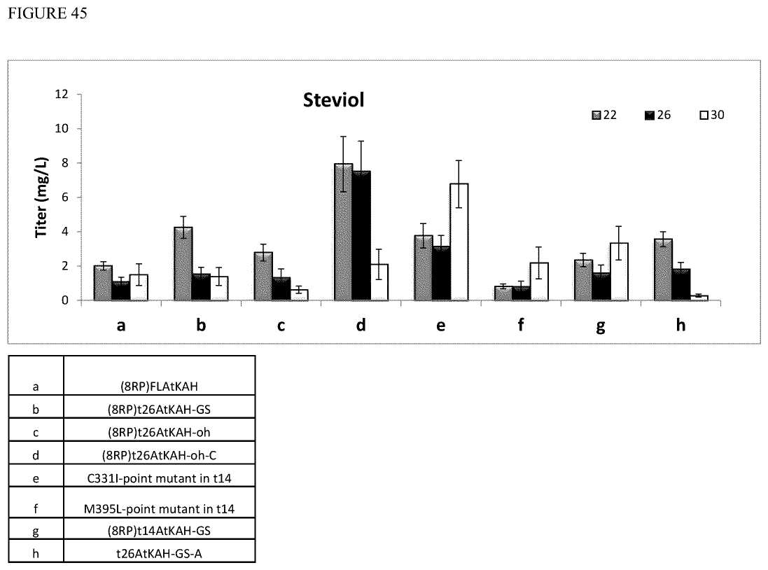

Mutations in AtKAH were screened for activity at 22, 26, and 30° C. C331I provided substantial thermostability, as shown in FIG. 45. C331I was made in the t14 background.

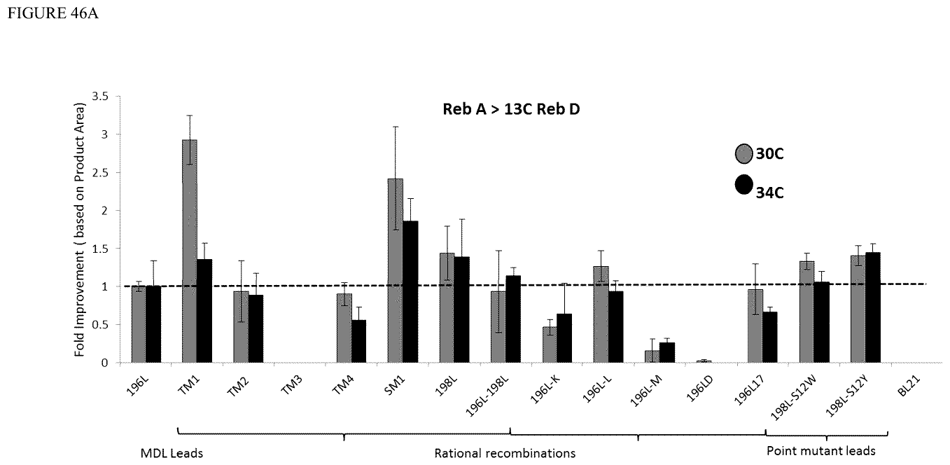

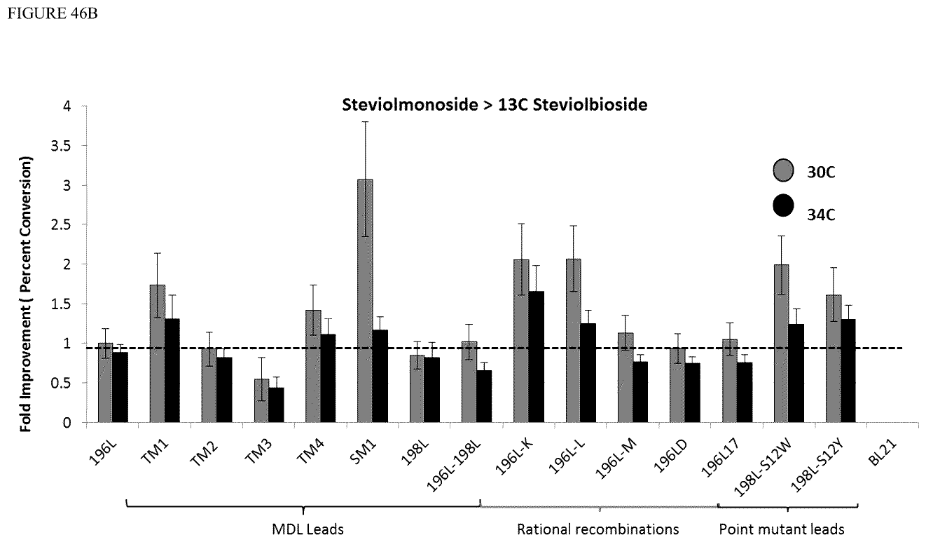

MbUGT1,2 rational recombinations were made, and screened at 30 and 34° C. for conversion of Reb A to Reb D (FIG. 46A), as well as for conversion of Steviolmonoside to 13c Steviolbioside (FIG. 46B). These studies resulted in a circular permutant truncated to create a new N-terminus at residue 196, with mutations introduced at S16W, H422E, R430E, R434H (MbUGT1,2-2). In these studies, cells producing these enzymes were induced for 4 hours of protein production at the listed temperature, extracted, and assayed in vitro overnight. Substrate concentration is 1 mM.

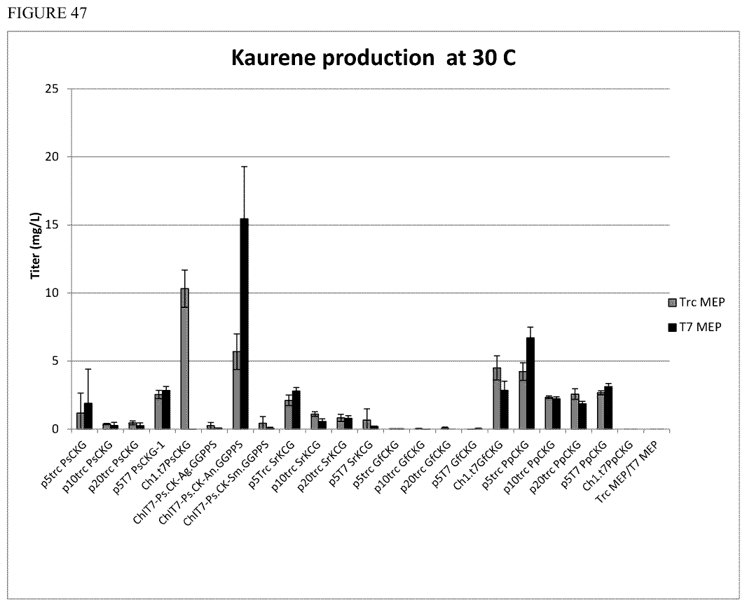

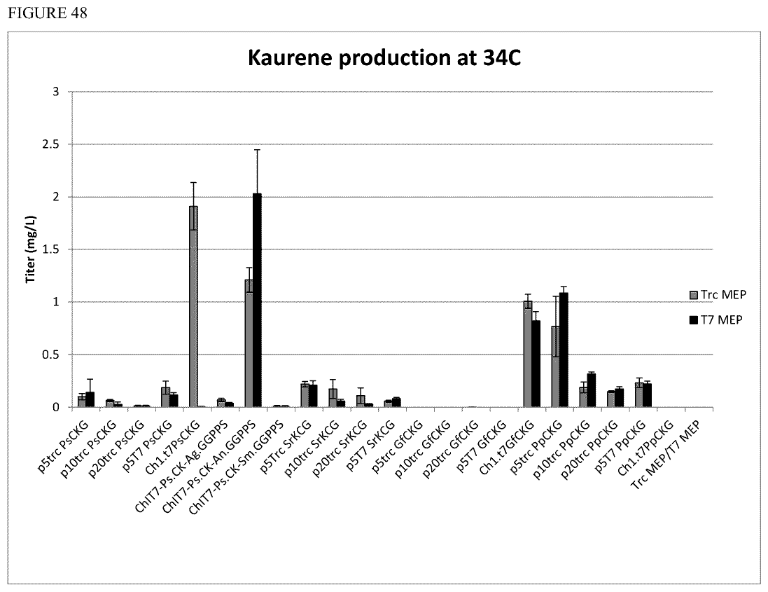

The effect of temperature on kaurene substrate production at 30 and 34° C. was tested. FIG. 47 shows kaurene production at 30° C. across various module constructs and FIG. 48 shows kaurene production at 34° C. across various module constructs. At 30° C., Ch1.T7-PsCK-AnGGPPS in T7MEP background gave highest kaurene titers (˜15 mg/L). At 34° C., Ch1.T7-PsCK-AnGGPPS in T7MEP background showed the highest kaurene titers (˜2 mg/L).

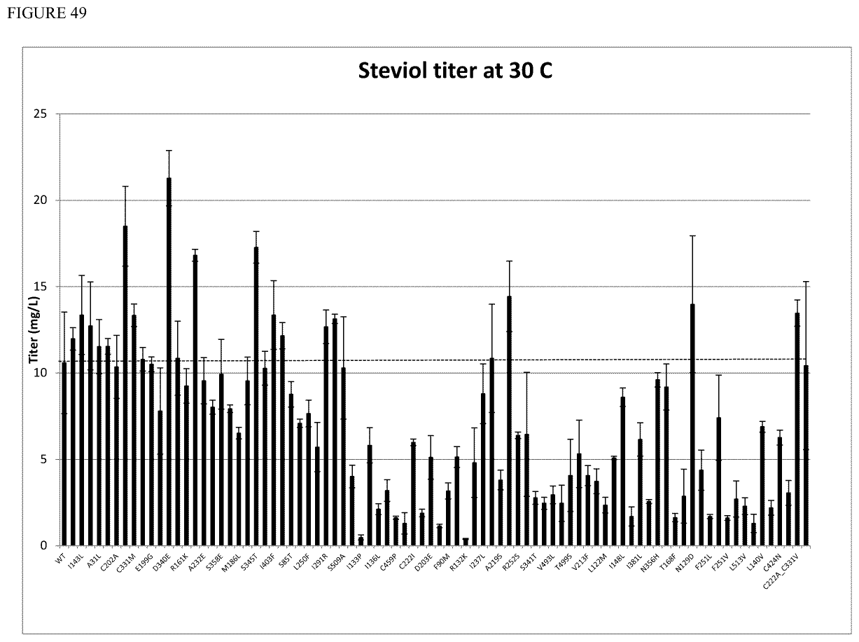

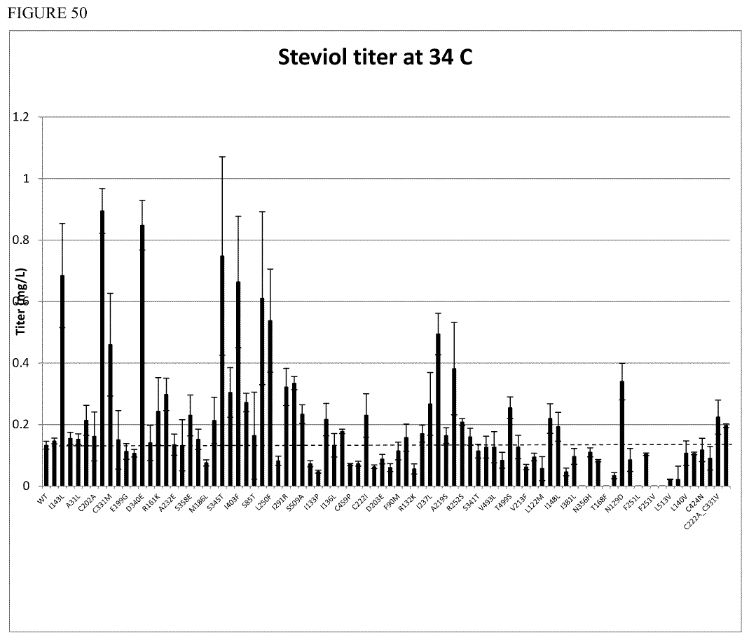

To investigate the thermotolerance of AtKAH, AtKAH point mutants were tested at 30° C., and 34° C. Conditions were: R media+glucose, 96 deep well plate, 3 days at 30° ° C. or 34° C. The strain background was p5Trc-(8RP)t14AtKAH-O-(8RP)t20SrKO-O-FLSrCPR. FIG. 49 shows production of Steviol at 30° C. across a library of AtKAH point mutations. FIG. 50 shows production of Steviol at 34° C. across a library of AtKAH point mutations. Various point mutations show improved thermotolerance that wild type, as shown by higher titers of steviol at 30 or 34° C.

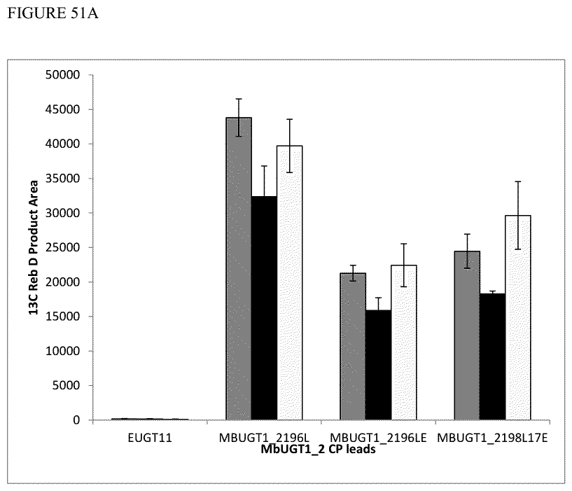

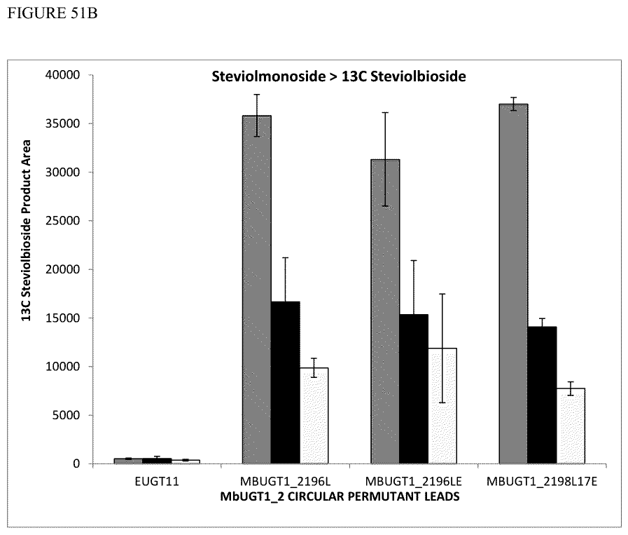

MbUGT1_2 curcular permutants were tested for activity at 30, 33, and 37° ° C. FIG. 51A and FIG. 51B shows activities of MbUGT1_2 circular permutants at 30° C., 34° C., and 37° C. Panel (A) shows conversion of Reb A to Reb D, while Panel (B) shows conversion of Steviolmonoside to 13C Steviolbioside. For both, expression of circular permutants was induced, followed by a four hour incubation period. As shown, EUGT11 lost its activity when induced at and above 30° C. In contrast, lead circular permutants seem to be most active at 30° C. MbUGT1_2 196 L retains highest activity on both substrates.

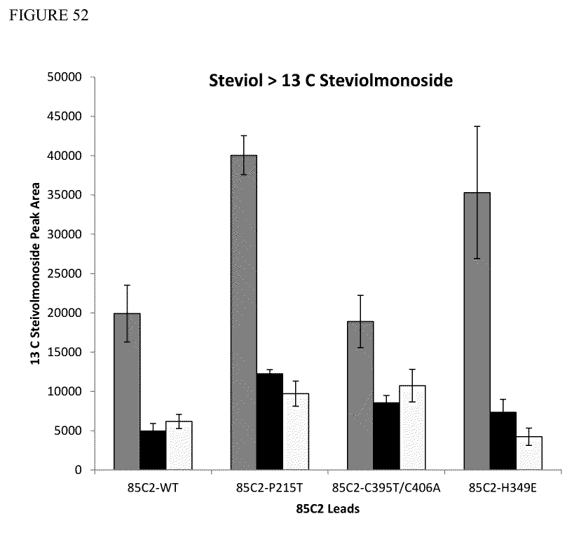

FIG. 52 shows activities of UGT85C2 mutants for conversion of Steviol to 13C Steviolmonoside at 30° C., 34° C., and 37° C. Expression was induced, followed by a four hour incubation period. As shown, 85C2-WT and the leads retain comparable activity at 34° C. and 37° C., maintaining highest activity at 30° C.

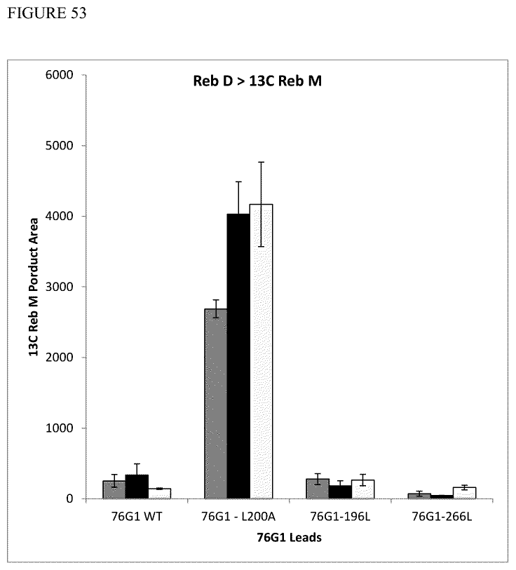

FIG. 53 shows activities of UGT76G1 mutants for conversion of Reb D to 13C Reb M at 30° C., 34° C., and 37° C. Expression was induced, followed by a four hour incubation period. 76G1-L200A is particularly active when induced and assayed at the higher temperatures, possibly due to a greater amount of protein.

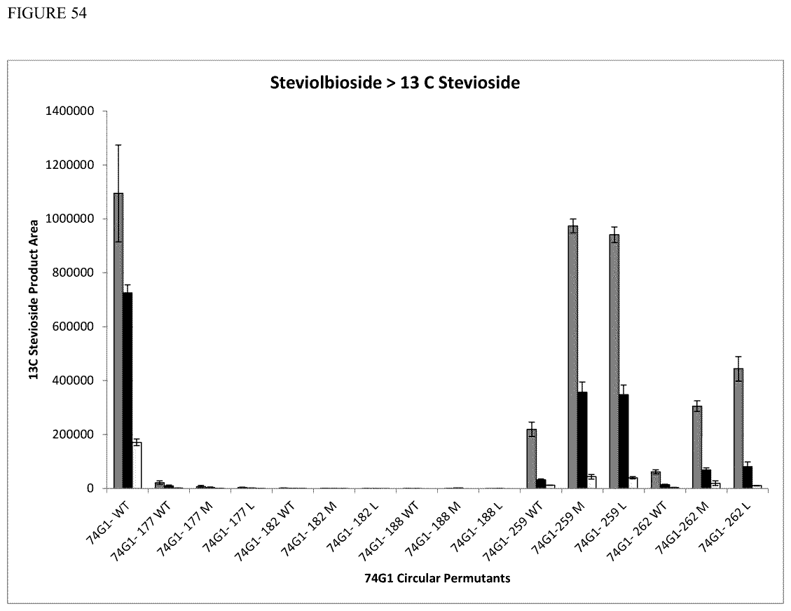

FIG. 54 shows activities of UGT74G1 circular permutants for conversion of Steviolbioside to 13C Stevioside at 30° C., 34° C., and 37° C. 74G1-WT retains activity on Steviolbioside even when induced and assayed at 37 C. The circular permutants 74G1-259M and 74G1-259L show a significant drop in activity at higher temperatures.

### REFERENCES

1. Sharma M, Thakral N K, & Thakral S (2009) Chemistry and in vivo profile of ent-kaurene glycosides of Stevia rebaudiana Bertoni: An overview. Natural Product Radiance 8(2): 181-189.
2 Chang M C Y & Keasling J D (2006) Production of isoprenoid pharmaceuticals by engineered microbes. Nature chemical biology 2(12):674-681.
3. Ajikumar P K, et al. (2008) Terpenoids: opportunities for biosynthesis of natural product drugs using engineered microorganisms. Molecular pharmaceutics 5(2): 167-190.
4. Ajikumar P K, et al. (2010) Isoprenoid pathway optimization for Taxol precursor overproduction in Escherichia coli. Science 330(6000):70-74.
5. Dugar D & Stephanopoulos G (2011) Relative potential of biosynthetic pathways for biofuels and bio-based products. Nature Biotechnology 29(12): 1074-1078.
6. Leonard E, et al. (2010) Combining metabolic and protein engineering of a terpenoid biosynthetic pathway for overproduction and selectivity control. Proceedings of the National Academy of Sciences 107(31): 13654-13659.
7. Yadav V G, De Mey M, Giaw Lim C, Kumaran Ajikumar P, & Stephanopoulos G (2012) The future of metabolic engineering and synthetic biology: Towards a systematic practice. Metabolic Engineering.
8. Yadav S K & Guleria P (2012) Steviol glycosides from Stevia: Biosynthesis pathway review and their application in foods and medicine. Critical Reviews in Food Science and Nutrition 52(11):988-998.
9. Richman A S, Gijzen M, Starratt A N, Yang Z, & Brandle J E (1999) Diterpene synthesis in Stevia rebaudiana: recruitment and up-regulation of key enzymes from the gibberellin biosynthetic pathway. The Plant Journal 19(4): 411-421.
10. Hayashi K, et al. (2006) Identification and functional analysis of bifunctional ent-kaurene synthase from the moss Physcomitrella patens. FEBS letters 580(26):6175-6181.
11. Toyomasu T, et al. (2000) Cloning of a full-length cDNA encoding ent-kaurene synthase from Gibberella fujikuroi: functional analysis of a bifunctional diterpene cyclase. Bioscience, biotechnology, and biochemistry 64(3):660-664.
12. Helliwell C A, Poole A, Peacock W J, & Dennis E S (1999) Arabidopsis ent-kaurene oxidase catalyzes three steps of gibberellin biosynthesis. Plant Physiology 119(2):507-510.
13. Brandle J & Telmer P (2007) Steviol glycoside biosynthesis. Phytochemistry 68(14):1855-1863.
14. Richman A, Swanson A, Humphrey T, Chapman R, McGarvey B, Pocs R & Brandle J. (2005) Functional genomics uncovers three glucosyltransferases involved in the synthesis of the major sweet glucosides of Stevia rebaudiana. The Plant Journal 41(1):56-67.
15. Hefner J, Ketchum R E B, & Croteau R (1998) Cloning and Functional Expression of a cDNA Encoding Geranylgeranyl Diphosphate Synthase from Taxus canadensis and Assessment of the Role of this Prenyltransferase in Cells Induced for Taxol Production. Archives of biochemistry and biophysics 360(1): 62-74.
16. Burke C & Croteau R (2002) Interaction with the small subunit of geranyl diphosphate synthase modifies the chain length specificity of geranylgeranyl diphosphate synthase to produce geranyl diphosphate. Journal of Biological Chemistry 277(5): 3141-3149.
17. Galagan J E, et al. (2005) Sequencing of Aspergillus nidulans and comparative analysis with A. fumigatus and A, oryzae. Nature 438(7071): 1105-1115.
18. Kim S Y, et al. (2009) Cloning and heterologous expression of the cyclooctatin biosynthetic gene cluster afford a diterpene cyclase and two P450 hydroxylases. Chemistry & biology 16(7):736-743.
19. Kawaide H, Imai R, Sassa T, & Kamiya Y (1997) ent-Kaurene Synthase from the Fungus Phaeosphaeria sp. L487. Journal of Biological Chemistry 272(35):21706-21712.
20. Humphrey T V, Richman A S, Menassa R, & Brandle J E (2006) Spatial organisation of four enzymes from Stevia rebaudiana that are involved in steviol glycoside synthesis. Plant molecular biology 61(1):47-62.
21. Miyazaki S, Katsumata T, Natsume M, & Kawaide H (2011) The CYP701B1 of Physcomitrella patens is an ent-kaurene oxidase that resists inhibition by uniconazole-P. FEBS letters 585(12): 1879-1883.
22. Mizutani M & Ohta D (1998) Two Isoforms of NADPH: Cytochrome P450 Reductase in Arabidopsis thaliana: Gene Structure, Heterologous Expression in Insect Cells, and Differential Regulation. Plant Physiology 116(1):357-367.
23. Yamaguchi S, Nomura T, Magome H, Kamiya Y (2008) Method for producing steviol synthetase gene and steviol. US Patent Application Publication No. 2008/0271205.
24. Kim K K, Sawa Y, & Shibata H (1996) Hydroxylation of ent-Kaurenoic Acid to Steviol in Stevia rebaudiana Bertoni-Purification and Partial Characterization of the Enzyme. Archives of biochemistry and biophysics 332(2):223-230.
25. Schuler M A (1996) Plant cytochrome P450 monooxygenases. Critical reviews in plant sciences 15(3):235-284.
26. Heinemann U & Hahn M. (1995) Circular permutation of polypeptide chains: implications for protein folding and stability. Prog. Biophys. Molec. Biol. 64(2-3): 121-143.
27. Yu Y, Lutz S (2011) Circular permutation: a different way to engineer enzyme structure and function. Trends Biotechnol 29(1): 18-25.
28. Houghton-Larsen J, et al. Recombinant production of steviol glycosides. PCT Publication No. WO 2013/022989.

TABLE 1
Summary of enzyme/gene sequences enabling biosynthesis of steviol.

| No. | Enzyme | Species | Gene ID | Protein ID |
|---|---|---|---|---|
| 1 | TcGGPPS | Taxus canadensis | AF081514.1 | AAD16018.1 |
| 2 | AgGGPPS | Abies grandis | AF425235.2 | AAL17614.2 |
| 3 | AnGGPPS | Aspergillus nidulans | XM_654104.1 | XP_659196.1 |
| 4 | SmGGPPS | Streptomyces melanosporofaciens | AB448947.1 | BAI44337.1 |
| 5 | MbGGPPS | Marine bacterium 443 | n/a | AAR37858.1 |
| 6 | PhGGPPS | Paracoccus haeundaensis | n/a | AAY28422.1 |
| 7 | CtGGPPS | Chlorobium tepidum TLS | NC_002932.3 | NP_661160.1 |
| 8 | SsGGPPS | Synechococcus sp. JA-3-3Ab | n/a | ABC98596.1 |
| 9 | Ss2GGPPS | Synechocystis sp. PCC 6803 | n/a | BAA16690.1 |
| 10 | TmGGPPS | Thermotoga maritima HB8 | n/a | NP_227976.1 |
| 11 | CgGGPPS | Corynebacterium glutamicum | n/a | NP_601376.2 |
| 12 | TtGGPPS | Thermus thermophillus HB27 | n/a | YP_143279.1 |
| 13 | PcGGPPS | Pyrobaculum calidifontis JCM 11548 | n/a | WP_011848845.1 |
| 14 | SrCPPS | Stevia rebaudiana | AF034545.1 | AAB87091.1 |
| 15 | EtCPPS | Erwina tracheiphila | n/a | WP_020322919.1 |
| 16 | SfCPPS | Sinorhizobium fredii | n/a | WP_010875301.1 |
| 17 | SrKS | Stevia rebaudiana | AF097311.1 | AAD34295.1 |
| 18 | EtKS | Erwina tracheiphila | n/a | WP_020322918.1 |
| 19 | SfKS | Sinorhizobium fredii | n/a | WP_010875302.1 |
| 20 | GfCPPS/KS | Gibberella fujikuroi | AB013295.1 | Q9UVY5.1 |
| 21 | PpCPPS/KS | Physcomitrella patens | AB302933.1 | BAF61135.1 |
| 22 | PsCPPS/KS | Phaeosphaeria sp. L487 | AB003395.1 | O13284.1 |
| 23 | AtKO | Arabidopsis thaliana | NM_122491.2 | NP_197962.1 |
| 24 | SrKO | Stevia rebaudiana | AY364317.1 | AAQ63464.1 |
| 25 | PpKO | Physcomitrella patens | AB618673.1 | BAK19917.1 |
| 26 | AtCPR | Arabidopsis thaliana | X66016.1 | CAA46814.1 |
| 27 | SrCPR | Stevia rebaudiana | DQ269454.4 | ABB88839.2 |
| 28 | AtKAH | Arabidopsis thaliana | NM_122399.2 | NP_197872.1 |
| 29 | SrKAH1 | Stevia rebaudiana | DQ398871.3 | ABD60225.1 |
| 30 | SrKAH2 | Stevia rebaudiana | n/a | n/a |

TABLE 2
Strains constructed to evaluate pathways for kaurene biosynthesis.

| Strain # | Upstream | Downstream |
|---|---|---|
| 1 | WT | Ch1.T7-KCG |
| 2 | Ch1.Trc-MEP | Ch1.T7-KCG |
| 3 | Ch1.T7-MEP | Ch1.T7-KCG |
| 4 | WT | p5-Trc-KCG |
| 5 | Ch1.Trc-MEP | p5-Trc-KCG |
| 6 | Ch1.T7-MEP | p5-Trc-KCG |
| 7 | WT | p10-Trc-KCG |
| 8 | Ch1.Trc-MEP | p10-Trc-KCG |
| 9 | Ch1.T7-MEP | p10-Trc-KCG |
| 10 | WT | p20-Trc-KCG |
| 11 | Ch1.Trc-MEP | p20-Trc-KCG |
| 12 | Ch1.T7-MEP | p20-Trc-KCG |
| 13 | WT | p5-T7-KCG |
| 14 | Ch1.Trc-MEP | p5-T7-KCG |
| 15 | Ch1.T7-MEP | p5-T7-KCG |
| 16 | WT | Ch1.T7-PpCKG |
| 17 | Ch1.Trc-MEP | Ch1.T7-PpCKG |
| 18 | Ch1.T7-MEP | Ch1.T7-PpCKG |
| 19 | WT | p5-Trc-PpCKG |
| 20 | Ch1.Trc-MEP | p5-Trc-PpCKG |
| 21 | Ch1.T7-MEP | p5-Trc-PpCKG |
| 22 | WT | p10-Trc-PpCKG |
| 23 | Ch1.Trc-MEP | p10-Trc-PpCKG |
| 24 | Ch1.T7-MEP | p10-Trc-PpCKG |
| 25 | WT | p20-Trc-PpCKG |
| 26 | Ch1.Trc-MEP | p20-Trc-PpCKG |
| 27 | Ch1.T7-MEP | p20-Trc-PpCKG |
| 28 | WT | p5-T7-PpCKG |
| 29 | Ch1.Trc-MEP | p5-T7-PpCKG |
| 30 | Ch1.T7-MEP | p5-T7-PpCKG |
| 31 | WT | Ch1.T7-GfCKG |
| 32 | Ch1.Trc-MEP | Ch1.T7-GfCKG |
| 33 | Ch1.T7-MEP | Ch1.T7-GfCKG |
| 34 | WT | p5-Trc-GfCKG |
| 35 | Ch1.Trc-MEP | p5-Trc-GfCKG |
| 36 | Ch1.T7-MEP | p5-Trc-GfCKG |
| 37 | WT | p10-Trc-GfCKG |
| 38 | Ch1.Trc-MEP | p10-Trc-GfCKG |
| 39 | Ch1.T7-MEP | p10-Trc-GfCKG |
| 40 | WT | p20-Trc-GfCKG |
| 41 | Ch1.Trc-MEP | p20-Trc-GfCKG |
| 42 | Ch1.T7-MEP | p20-Trc-GfCKG |
| 43 | WT | p5-T7-GfCKG |
| 44 | Ch1.Trc-MEP | p5-T7-GfCKG |
| 45 | Ch1.T7-MEP | p5-T7-GfCKG |
| 46 | WT | Ch1.T7-PsCKG |
| 47 | Ch1.Trc-MEP | Ch1.T7-PsCKG |
| 48 | Ch1.T7-MEP | Ch1.T7-PsCKG |
| 49 | WT | p5-Trc-PsCKG |
| 50 | Ch1.Trc-MEP | p5-Trc-PsCKG |
| 51 | Ch1.T7-MEP | p5-Trc-PsCKG |
| 52 | WT | p10-Trc-PsCKG |
| 53 | Ch1.Trc-MEP | p10-Trc-PsCKG |
| 54 | Ch1.T7-MEP | p10-Trc-PsCKG |
| 55 | WT | p20-Trc-PsCKG |
| 56 | Ch1.Trc-MEP | p20-Trc-PsCKG |
| 57 | Ch1.T7-MEP | p20-Trc-PsCKG |
| 58 | WT | p5-T7-PsCKG |
| 59 | Ch1.Trc-MEP | p5-T7-PsCKG |
| 60 | Ch1.T7-MEP | p5-T7-PsCKG |

TABLE 3
Combinations of upstream and downstream pathway configurations tested
for KO activity and kaurenoic acid biosynthesis. Ch1 = 1 copy
chromosomally integrated, p5/p10/p20 = plasmids of increasing
copy number, Trc/T7 = promoters of increasing transcriptional strength.

| | Upstream/Downstream | | |
|---|---|---|---|
| | Ch1.T7-MEP | Ch1.T7-MEP | Ch1.T7-MEP |
| | Ch1.T7-SrKCG | p10Trc-SrKCG | p20Trc-SrKCG |
| p5Trc-(8RP)t4SrKO-L-t69SrCPR | ✓ | ✓ | ✓ |
| p5Trc-(8RP)t20SrKO-L-t69SrCPR | ✓ | ✓ | ✓ |
| p5Trc-(8RP)t39SrKO-L-t69SrCPR | ✓ | ✓ | ✓ |
| p5Trc-(8RP)t39SrKO-(8RP)t69SrCPR | ✓ | ✓ | ✓ |
| p5Trc-(MA)t39SrKO-(8RP)t69SrCPR | ✓ | ✓ | ✓ |

TABLE 4
Fold-change in in vivo activity over parental enzyme for
point mutants of SrKO. The fold increases describe the
change in kaurene remaining in this strain, or the change
in kaurenoic acid produced, both relative to the wild-
type (non-mutated) enzyme-bearing parental strain.

| Wild-type | | | Fold increase | Fold increase |
|---|---|---|---|---|
| residue | Position | Mutation | in kaurene | in kaurenoic acid |
| A | 116 | R | 1.0 | 1.8 |
| T | 119 | S | 0.9 | 1.9 |
| I | 183 | V | 1.0 | 1.7 |
| H | 382 | Y | 1.0 | 1.8 |

TABLE 5
Combinations of upstream and downstream pathway configurations
tested for KO/KAH activity and steviol biosynthesis. Ch1
= 1 copy chromosomally integrated, p5 and p10 =
plasmids of increasing copy number, Trc/T7 = promoters
of increasing transcriptional strength.

| | | Steviol |
|---|---|---|
| Expression module | | Detected |
| Ch1.T7-MEP | p5Trc-(8RP)t39SrKO-(8RP)t7SrKAH | ++ |
| Ch1.T7- | p5Trc-(8RP)t39SrKO-(8RP)t21SrKAH | ++ |
| (8RP)t69SrCPR | | |
| p10Trc-SrKCG | p5Trc-(8RP)t39SrKO-(8RP)t29SrKAH | + |

TABLE 6
Fold-change in activity over parental enzyme for point mutants of AtKAH.
The fold increases describe the change in kaurenoic acid remaining
in this strain, or the change in steviol produced, both relative to
the wild-type (non-mutated) enzyme-bearing parental strain.

| Wild- | | | Wild- | | | | |
|---|---|---|---|---|---|---|---|
| type | | | type | | | Fold increase | Fold increase |
| residue | Position | Mutation | residue | Position | Mutation | kaurenoic acid | steviol |
| A | 25 | L | | | | 0.7 | 1.6 |
| K | 37 | R | | | | 0.8 | 1.4 |
| S | 79 | T | | | | 0.7 | 1.6 |
| F | 84 | I | | | | 1.3 | 0.0 |
| F | 84 | M | | | | 1.2 | 0.2 |
| Y | 95 | F | | | | 1.1 | 1.0 |
| H | 104 | I | | | | 1.4 | 0.0 |
| I | 107 | M | | | | 1.0 | 1.2 |
| L | 116 | M | | | | 1.4 | 0.9 |
| T | 119 | C | | | | 0.7 | 1.5 |
| N | 123 | D | | | | 0.9 | 1.1 |
| R | 126 | K | | | | 1.4 | 0.0 |
| I | 127 | P | | | | 1.1 | 0.0 |
| I | 127 | V | | | | 1.1 | 0.1 |
| I | 130 | L | | | | 1.0 | 0.0 |
| L | 134 | V | | | | 1.3 | 0.7 |
| I | 137 | L | | | | 0.3 | 1.9 |
| I | 142 | L | | | | 1.2 | 0.8 |
| I | 142 | V | | | | 0.5 | 2.1 |
| I | 143 | L | | | | 1.7 | 0.0 |
| R | 155 | K | | | | 0.9 | 2.0 |
| T | 162 | F | | | | 1.4 | 0.0 |
| H | 163 | M | | | | 1.2 | 0.2 |
| I | 166 | V | | | | 0.3 | 1.2 |
| M | 180 | L | | | | 1.3 | 1.5 |
| V | 188 | I | | | | 1.0 | 1.1 |
| E | 193 | G | | | | 1.1 | 1.7 |
| C | 196 | A | | | | 0.9 | 1.5 |
| D | 197 | E | | | | 1.7 | 0.8 |
| V | 207 | F | | | | 1.4 | 0.5 |
| A | 213 | S | | | | 0.7 | 0.9 |
| C | 216 | A | C | 325 | V | 0.8 | 0.9 |
| C | 216 | I | | | | 1.1 | 1.0 |
| C | 216 | S | | | | 1.3 | 0.0 |
| A | 226 | E | | | | 0.9 | 1.5 |
| I | 231 | L | | | | 0.3 | 0.8 |
| L | 235 | Q | | | | 0.3 | 2.4 |
| I | 238 | M | | | | 1.5 | 0.2 |
| L | 244 | F | | | | 0.9 | 1.4 |
| F | 245 | L | | | | 1.6 | 0.0 |
| F | 245 | V | | | | 1.5 | 0.0 |
| R | 246 | S | | | | 1.1 | 0.8 |
| F | 247 | L | | | | 1.0 | 1.1 |
| L | 272 | I | | | | 1.7 | 0.0 |
| S | 274 | D | | | | 1.0 | 1.3 |
| S | 275 | L | | | | 0.6 | 1.2 |
| I | 285 | R | | | | 0.6 | 1.7 |
| C | 287 | S | | | | 0.7 | 1.7 |
| K | 292 | E | | | | 1.2 | 0.6 |
| Q | 297 | E | | | | 1.0 | 0.9 |
| C | 307 | S | | | | 0.6 | 0.0 |
| V | 322 | I | | | | 1.1 | 1.4 |
| C | 325 | I | | | | 0.5 | 2.4 |
| C | 325 | M | | | | 0.6 | 2.2 |
| F | 330 | L | | | | 1.0 | 1.5 |
| D | 334 | E | | | | 0.3 | 2.2 |
| S | 335 | T | | | | 0.7 | 0.5 |
| S | 339 | T | | | | 0.2 | 2.2 |
| N | 350 | H | | | | 0.9 | 1.1 |
| S | 352 | E | | | | 0.7 | 1.5 |
| S | 363 | E | | | | 0.8 | 1.3 |
| E | 373 | D | | | | 1.1 | 1.6 |
| I | 375 | L | | | | 1.2 | 0.8 |
| V | 381 | L | | | | 1.3 | 0.7 |
| M | 389 | L | | | | 1.2 | 0.9 |
| I | 397 | F | | | | 0.8 | 1.7 |
| C | 418 | N | | | | 1.4 | 0.5 |
| S | 446 | A | | | | 0.6 | 1.2 |
| E | 447 | N | | | | 0.8 | 1.4 |
| C | 453 | P | | | | 0.7 | 0.0 |
| I | 460 | M | | | | 1.3 | 0.2 |
| V | 470 | L | | | | 0.7 | 1.7 |
| G | 475 | A | | | | 1.1 | 1.3 |
| M | 477 | V | | | | 0.8 | 0.6 |
| V | 487 | L | | | | 1.3 | 1.0 |
| T | 493 | S | | | | 1.1 | 1.2 |
| T | 497 | N | | | | 0.5 | 1.0 |
| Q | 499 | V | | | | 0.6 | 1.6 |
| S | 503 | A | | | | 0.3 | 1.0 |
| H | 504 | F | | | | 1.1 | 0.2 |
| K | 505 | R | | | | 0.5 | 0.9 |
| L | 506 | M | | | | 1.6 | 0.0 |
| L | 507 | I | | | | 1.5 | 0.0 |
| L | 507 | T | | | | 1.6 | 0.0 |
| L | 507 | V | | | | 1.5 | 0.0 |

TABLE 7
Modifications to E. coli strain to improve UDP-glucose substrate
pools and support high-titer production of steviol glycosides.

| | | Gene ID |
|---|---|---|
| Modification | Type | (BioCyc) |
| ΔgalE | Deletion | EG10362 |
| ΔgalT | Deletion | EG10366 |
| ΔgalK | Deletion | EG10363 |
| ΔgalM | Deletion | EG11698 |
| ΔushA | Deletion | EG11060 |
| Δagp | Deletion | EG10033 |
| Δpgm | Deletion | EG12144 |
| galU (Escherichia coli K-12 substr. MG1655) | Insertion | EG11319 |
| ugpA (Bifidobacterium bifidum PRL2010) | Insertion | BBPR_0976 |
| spl (Bifidobacterium adolescentis ATCC | Insertion | BAD_0078 |
| 15703) | | |

TABLE 8
Enzymes known to catalyze reactions required for steviol glycoside biosynthesis [to RebM].

| | | Type of | | | | |
|---|---|---|---|---|---|---|
| Substrate | Product | glycosylation | Enzyme 1 | Enzyme 2 | Enzyme 3 | Enzyme 4 |
| Steviol | Steviolmonoside | C13 | SrUGT85C2 | | | |
| Steviol | C19-Glu-Steviol | C19 | SrUGT74G1 | MbUGTc19 | | |
| Steviolmonoside | Steviolbioside | 1-2′ | SrUGT91D1 | SrUGT91D2 | OsUGT1-2 | MbUGT1-2 |
| Steviolmonoside | Rubusoside | C19 | SrUGT74G1 | MbUGTc19 | | |
| C19-Glu-Steviol | Rubusoside | C13 | SrUGT85C2 | | | |
| Steviolbioside | Stevioside | C19 | SrUGT74G1 | MbUGTc19 | | |
| Steviolbioside | RebB | 1-3′ | SrUGT76G1 | | | |
| Stevioside | RebE | 1-2′ | SrUGT91D1 | SrUGT91D2 | OsUGT1-2 | MbUGT1-2 |
| Stevioside | RebA | 1-3′ | SrUGT76G1 | | | |
| RebB | RebA | C19 | SrUGT74G1 | MbUGTc19 | | |
| RebE | RebD | 1-3′ | SrUGT76G1 | | | |
| RebA | RebD | 1-2′ | SrUGT91D1 | SrUGT91D2 | OsUGT1-2 | MbUGT1-2 |
| RebD | RebM | 1-3′ | SrUGT76G1 | | | |

TABLE 9
Summary of enzyme/gene sequences for biosynthesis
of steviol glycosides, including RebM.

| Type of | | | | |
|---|---|---|---|---|
| glycosylation | Enzyme | Gene ID | Protein ID | Description |
| C13 | SrUGT85C2 | AY345978.1 | AAR06916.1 | |
| C19 | SrUGT74G1 | AY345982.1 | AAR06920.1 | |
| | MbUGTc19 | — | — | circular permutant of |
| | | | | SrUGT74G1 |
| 1-2′ | SrUGT91D1 | AY345980.1 | AAR06918.1 | |
| | SrUGT91D2 | ACE87855.1 | ACE87855.1 | |
| | SrUGT91D2e | | | US2011/038967 |
| | OsUGT1-2 | NM_001057542.1 | NP_001051007.2 | WO 2013/022989 |
| | MbUGT1-2 | — | — | circular permutant of |
| | | | | OsUGT1-2 |
| 1-3′ | SrUGT76G1 | FB917645.1 | CAX02464.1 | |

TABLE 10
Summary of steviol glycoside structures.

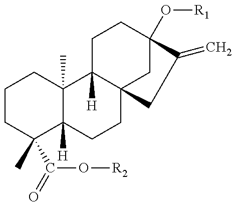

| Symbol | Common Name | R1 | R2 | Glycosylations |
|---|---|---|---|---|
| SG1 | Steviolmonoside | Glcβ1- | H— | 1 |
| SG2 | C19-glucopyranosyl | H— | Glcβ1- | 1 |
| | steviol | | | |
| SG3 | Steviolbioside | Glcβ1-2Glcβ1- | H— | 2 |
| SG4 | — | H— | Glcβ1-2Glcβ1- | 2 |
| SG5 | Rubusoside | Glcβ1- | Glcβ1- | 2 |
| SG6 | — | Glcβ1-3Glcβ1- | H— | 2 |
| SG7 | — | H— | Glcβ1-3Glcβ1- | 2 |
| SG8 | Stevioside | Glcβ1-2Glcβ1- | Glcβ1- | 3 |
| SG9 | Rebaudioside G | Glcβ1-3Glcβ1- | Glcβ1- | 3 |
| SG10 | — | Glcβ1- | Glcβ1-2Glcβ1- | 3 |
| SG11 | — | Glcβ1- | Glcβ1-3Glcβ1- | 3 |
| SG12 | Rebaudioside B | Glcβ1-2(Glcβ1-3)Glcβ1- | H— | 3 |
| SG13 | — | H— | Glcβ1-2(Glcβ1-3)Glcβ1- | 3 |
| SG14 | Rebaudioside E | Glcβ1-2Glcβ1- | Glcβ1-2Glcβ1- | 4 |
| SG15 | — | Glcβ1-3Glcβ1- | Glcβ1-3Glcβ1- | 4 |
| SG16 | Rebaudioside A | Glcβ1-2(Glcβ1-3)Glcβ1- | Glcβ1- | 4 |
| SG17 | — | Glcβ1- | Glcβ1-2(Glcβ1-3)Glcβ1- | 4 |
| SG18 | — | Glcβ1-3Glcβ1-2(Glcβ1- | H— | 4 |
| | | 3)Glcß1- | | |
| SG19 | — | H— | Glcβ1-3Glcβ1-2(Glcβ1- | 4 |
| | | | 3)Glcβ1- | |
| SG20 | — | Glcβ1-2Glcβ1- | Glcβ1-3Glcβ1- | 4 |
| SG21 | — | Glcβ1-3Glcβ1- | Glcβ1-2Glcβ1- | 4 |
| SG22 | — | Glcβ1-2(Glcb1-2Glcβ1- | H— | 4 |
| | | 3)Glcβ1- | | |
| SG23 | — | H— | Glcβ1-2(Glcb1-2Glcβ1- | 4 |
| | | | 3)Glcβ1- | |
| SG24 | Rebaudioside D | Glcβ1-2(Glcβ1-3)Glcβ1- | Glcβ1-2Glcβ1- | 5 |
| SG25 | — | Glcβ1-2Glcβ1- | Glcβ1-2(Glcβ1-3)Glcβ1- | 5 |
| SG26 | Rebaudioside I | Glcβ1-2(Glcβ1-3)Glcβ1- | Glcβ1-3Glcβ1- | 5 |
| SG27 | — | Glcβ1-3Glcβ1- | Glcβ1-2(Glcβ1-3)Glcβ1- | 5 |
| SG28 | — | Glcβ1-3Glcβ1-2(Glcβ1- | Glcβ1- | 5 |
| | | 3)Glcβ1- | | |
| SG29 | — | Glcβ1- | Glcβ1-3Glcβ1-2(Glcβ1- | 5 |
| | | | 3)Glcβ1- | |
| SG30 | — | Glcβ1-2(Glcb1-2Glcβ1- | Glcβ1- | 5 |
| | | 3)Glcβ1- | | |
| SG31 | — | Glcβ1- | Glcβ1-2(Glcb1-2Glcβ1- | 5 |
| | | | 3)Glcβ1- | |
| SG32 | Rebaudioside M | Glcβ1-2(Glcβ1-3)Glcβ1- | Glcβ1-2(Glcβ1-3)Glcβ1- | 6 |
| SG33 | — | Glcβ1-3Glcβ1-2(Glcβ1- | Glcβ1-2Glcβ1- | 6 |
| | | 3)Glcβ1- | | |
| SG34 | — | Glcβ1-3Glcβ1-2(Glcβ1- | Glcβ1-3Glcβ1- | 6 |
| | | 3)Glcβ1- | | |
| SG35 | — | Glcβ1-2Glcβ1- | Glcβ1-3Glcβ1-2(Glcβ1- | 6 |
| | | | 3)Glcβ1- | |
| SG36 | — | Glcβ1-3Glcβ1- | Glcβ1-3Glcβ1-2(Glcβ1- | 6 |
| | | | 3)Glcβ1- | |
| SG37 | — | Glcβ1-2(Glcb1-2Glcβ1- | Glcβ1-2Glcβ1- | 6 |
| | | 3)Glcβ1- | | |
| SG38 | — | Glcβ1-2(Glcb1-2Glcβ1- | Glcβ1-3Glcβ1- | 6 |
| | | 3)Glcβ1- | | |
| SG39 | — | Glcβ1-2Glcβ1- | Glcβ1-2(Glcb1-2Glcβ1- | 6 |
| | | | 3)Glcβ1- | |
| SG40 | — | Glcβ1-3Glcβ1- | Glcβ1-2(Glcb1-2Glcβ1- | 6 |
| | | | 3)Glcβ1- | |
| SG41 | — | Glcβ1-3Glcβ1-2(Glcβ1- | Glcβ1-2(Glcβ1-3)Glcβ1- | 7 |
| | | 3)Glcβ1- | | |
| SG42 | — | Glcβ1-2(Glcβ1-3)Glcβ1- | Glcβ1-3Glcβ1-2(Glcβ1- | 7 |
| | | | 3)Glcβ1- | |
| SG43 | — | Glcβ1-2(Glcb1-2Glcβ1- | Glcβ1-2(Glcβ1-3)Glcβ1- | 7 |
| | | 3)Glcβ1- | | |
| SG44 | — | Glcβ1-2(Glcβ1-3)Glcβ1- | Glcβ1-2(Glcb1-2Glcβ1- | 7 |
| | | | 3)Glcβ1- | |
| SG45 | — | Glcβ1-3Glcβ1-2(Glcβ1- | Glcß1-3Glcß1-2(Glcß1- | 8 |
| | | 3)Glcβ1- | 3)Glcβ1- | |
| SG46 | — | Glcβ1-2(Glcb1-2Glcβ1- | Glcβ1-2(Glcb1-2Glcβ1- | 8 |
| | | 3)Glcβ1- | 3)Glcβ1- | |
| SG47 | — | Glcβ1-3Glcβ1-2(Glcβ1- | Glcβ1-3Glcβ1-2(Glcβ1- | 8 |
| | | 3)Glcβ1- | 3)Glcβ1- | |
| SG48 | — | Glcβ1-2(Glcb1-2Glcβ1- | Glcβ1-2(Glcb1-2Glcβ1- | 8 |
| | | 3)Glcβ1- | 3)Glcβ1- | |

TABLE 11
Fold-change in activity over parental enzyme for point
mutants of SrUGT85C2 (C13-O-glycosylating activity).

| WT | Pos. | Mutation | WT | Pos. | Mutation | WT | Pos. | Mutation | WT | Pos. | Mutation | [symbol] |
|---|---|---|---|---|---|---|---|---|---|---|---|---|
| D | 2 | G | | | | | | | | | | [symbol] |
| A | 3 | S | | | | | | | | | | [symbol] |
| V | 13 | A | | | | | | | | | | [symbol] |
| V | 13 | A | L | 40 | F | | | | | | | [symbol] |
| V | 13 | I | L | 40 | H | | | | | | | [symbol] |
| I | 14 | V | | | | | | | | | | [symbol] |
| F | 15 | C | | | | | | | | | | [symbol] |
| F | 18 | Y | | | | | | | | | | [symbol] |
| S | 22 | G | | | | | | | | | | [symbol] |
| S | 22 | G | A | 27 | P | | | | | | | [symbol] |
| K | 25 | N | | | | | | | | | | [symbol] |
| A | 26 | P | | | | | | | | | | [symbol] |
| Q | 32 | K | | | | | | | | | | [symbol] |
| K | 37 | R | | | | | | | | | | [symbol] |
| L | 39 | F | | | | | | | | | | [symbol] |
| Q | 40 | H | | | | | | | | | | [symbol] |
| Q | 40 | R | | | | | | | | | | [symbol] |
| D | 47 | E | | | | | | | | | | [symbol] |
| F | 48 | Y | | | | | | | | | | [symbol] |
| I | 49 | N | | | | | | | | | | [symbol] |
| N | 51 | K | | | | | | | | | | [symbol] |
| Q | 52 | R | | | | | | | | | | [symbol] |
| F | 53 | L | | | | | | | | | | [symbol] |
| E | 55 | K | | | | | | | | | | [symbol] |
| S | 57 | R | | | | | | | | | | [symbol] |
| H | 60 | N | | | | | | | | | | [symbol] |
| C | 61 | A | | | | | | | | | | [symbol] |
| A | 65 | L | | | | | | | | | | [symbol] |
| G | 67 | D | | | | | | | | | | [symbol] |
| V | 77 | L | | | | | | | | | | [symbol] |
| S | 78 | P | | | | | | | | | | [symbol] |
| H | 79 | P | | | | | | | | | | [symbol] |
| P | 81 | D | | | | | | | | | | [symbol] |
| A | 83 | D | | | | | | | | | | [symbol] |
| S | 84 | A | | | | | | | | | | [symbol] |
| I | 85 | T | | | | | | | | | | [symbol] |
| P | 86 | Q | | | | | | | | | | [symbol] |
| I | 87 | D | | | | | | | | | | [symbol] |
| R | 88 | I | | | | | | | | | | [symbol] |
| E | 89 | P | | | | | | | | | | [symbol] |
| L | 92 | C | | | | | | | | | | [symbol] |
| R | 93 | E | | | | | | | | | | [symbol] |
| I | 95 | T | | | | | | | | | | [symbol] |
| E | 96 | R | | | | | | | | | | [symbol] |
| T | 97 | K | | | | | | | | | | [symbol] |
| F | 99 | C | | | | | | | | | | [symbol] |
| F | 99 | L | F | 127 | W | | | | | | | [symbol] |
| D | 101 | A | | | | | | | | | | [symbol] |
| R | 102 | P | | | | | | | | | | [symbol] |
| I | 104 | E | | | | | | | | | | [symbol] |
| I | 104 | R | | | | | | | | | | [symbol] |
| I | 104 | R | K | 134 | E | | | | | | | [symbol] |
| V | 107 | L | | | | | | | | | | [symbol] |
| T | 108 | A | | | | | | | | | | [symbol] |
| P | 111 | N | | | | | | | | | | [symbol] |
| P | 114 | V | | | | | | | | | | [symbol] |
| I | 118 | V | | | | | | | | | | [symbol] |
| L | 123 | M | | | | | | | | | | [symbol] |
| I | 128 | L | | | | | | | | | | [symbol] |
| K | 132 | E | | | | | | | | | | [symbol] |
| K | 133 | E | | | | | | | | | | [symbol] |
| V | 138 | E | | | | | | | | | | [symbol] |
| V | 138 | R | | | | | | | | | | [symbol] |
| M | 139 | V | | | | | | | | | | [symbol] |
| M | 140 | L | | | | | | | | | | [symbol] |
| Y | 141 | F | | | | | | | | | | [symbol] |
| A | 145 | S | | | | | | | | | | [symbol] |
| F | 152 | Y | | | | | | | | | | [symbol] |
| Y | 153 | L | | | | | | | | | | [symbol] |
| I | 155 | Y | | | | | | | | | | [symbol] |
| H | 156 | R | | | | | | | | | | [symbol] |
| F | 163 | L | | | | | | | | | | [symbol] |
| A | 169 | E | | | | | | | | | | [symbol] |
| V | 184 | I | | | | | | | | | | [symbol] |
| E | 188 | K | | | | | | | | | | [symbol] |
| G | 189 | N | | | | | | | | | | [symbol] |
| F | 195 | L | | | | | | | | | | [symbol] |
| L | 197 | F | | | | | | | | | | [symbol] |
| D | 198 | I | | | | | | | | | | [symbol] |
| W | 199 | R | | | | | | | | | | [symbol] |
| S | 200 | T | | | | | | | | | | [symbol] |
| L | 203 | P | | | | | | | | | | [symbol] |
| K | 206 | I | | | | | | | | | | [symbol] |
| V | 207 | M | | | | | | | | | | [symbol] |
| M | 209 | N | | | | | | | | | | [symbol] |
| A | 214 | E | | | | | | | | | | [symbol] |
| P | 215 | T | | | | | | | | | | [symbol] |
| Q | 216 | E | | | | | | | | | | [symbol] |
| S | 218 | A | | | | | | | | | | [symbol] |
| V | 221 | A | | | | | | | | | | [symbol] |
| H | 223 | A | | | | | | | | | | [symbol] |
| H | 224 | I | | | | | | | | | | [symbol] |
| I | 225 | L | M | 472 | F | | | | | | | [symbol] |
| F | 226 | L | | | | | | | | | | [symbol] |
| H | 227 | N | | | | | | | | | | [symbol] |
| S | 235 | D | | | | | | | | | | [symbol] |
| I | 236 | V | | | | | | | | | | [symbol] |
| I | 237 | L | | | | | | | | | | [symbol] |
| K | 238 | D | | | | | | | | | | [symbol] |
| T | 239 | A | | | | | | | | | | [symbol] |
| L | 242 | S | | | | | | | | | | [symbol] |
| R | 243 | I | | | | | | | | | | [symbol] |
| Y | 244 | L | | | | | | | | | | [symbol] |
| N | 245 | P | | | | | | | | | | [symbol] |
| H | 246 | P | | | | | | | | | | [symbol] |
| I | 247 | V | | | | | | | | | | [symbol] |
| D | 258 | N | | | | | | | | | | [symbol] |
| F | 285 | L | | | | | | | | | | [symbol] |
| Q | 289 | D | | | | | | | | | | [symbol] |
| K | 291 | Q | Y | 326 | R | | | | | | | [symbol] |
| K | 291 | Q | E | 293 | P | Y | 326 | R | | | | [symbol] |
| E | 292 | P | Y | 326 | R | | | | | | | [symbol] |
| T | 304 | I | | | | | | | | | | [symbol] |
| S | 308 | T | | | | | | | | | | [symbol] |
| D | 311 | Q | | | | | | | | | | [symbol] |
| M | 312 | L | | | | | | | | | | [symbol] |
| M | 312 | L | I | 331 | V | | | | | | | [symbol] |
| G | 316 | A | | | | | | | | | | [symbol] |
| A | 320 | E | H | 350 | R | | | | | | | [symbol] |
| A | 320 | E | E | 346 | G | H | 350 | R | | | | [symbol] |
| N | 323 | G | | | | | | | | | | [symbol] |
| Y | 325 | P | | | | | | | | | | [symbol] |
| I | 329 | V | | | | | | | | | | [symbol] |
| S | 332 | P | | | | | | | | | | [symbol] |
| N | 333 | D | | | | | | | | | | [symbol] |
| N | 339 | S | | | | | | | | | | [symbol] |
| E | 345 | G | | | | | | | | | | [symbol] |
| E | 345 | G | H | 350 | R | | | | | | | [symbol] |
| L | 346 | F | | | | | | | | | | [symbol] |
| L | 346 | F | I | 351 | T | | | | | | | [symbol] |
| H | 349 | E | | | | | | | | | | [symbol] |
| K | 352 | D | | | | | | | | | | [symbol] |
| F | 355 | L | | | | | | | | | | [symbol] |
| S | 361 | P | | | | | | | | | | [symbol] |
| K | 364 | Q | | | | | | | | | | [symbol] |
| L | 375 | V | | | | | | | | | | [symbol] |
| L | 375 | V | I | 395 | V | | | | | | | [symbol] |
| L | 375 | V | I | 395 | V | L | 432 | A | L | 436 | V | [symbol] |
| G | 381 | N | | | | | | | | | | [symbol] |
| I | 384 | L | | | | | | | | | | [symbol] |
| L | 387 | I | | | | | | | | | | [symbol] |
| L | 387 | I | V | 416 | I | | | | | | | [symbol] |
| S | 388 | C | | | | | | | | | | [symbol] |
| I | 394 | V | L | 432 | A | | | | | | | [symbol] |
| I | 394 | V | L | 436 | V | | | | | | | [symbol] |
| C | 395 | A | | | | | | | | | | [symbol] |
| C | 395 | A | C | 407 | A | | | | | | | [symbol] |
| C | 395 | T | C | 407 | A | | | | | | | [symbol] |
| Y | 398 | F | | | | | | | | | | [symbol] |
| S | 399 | F | | | | | | | | | | [symbol] |
| W | 400 | A | | | | | | | | | | [symbol] |
| L | 403 | Q | | | | | | | | | | [symbol] |
| I | 409 | V | | | | | | | | | | [symbol] |
| E | 414 | G | | | | | | | | | | [symbol] |
| E | 414 | K | K | 443 | E | | | | | | | [symbol] |
| V | 415 | I | | | | | | | | | | [symbol] |
| L | 417 | M | | | | | | | | | | [symbol] |
| M | 419 | I | | | | | | | | | | [symbol] |
| G | 420 | D | | | | | | | | | | [symbol] |
| K | 422 | D | | | | | | | | | | [symbol] |
| D | 426 | E | | | | | | | | | | [symbol] |
| K | 429 | E | | | | | | | | | | [symbol] |
| K | 429 | E | Q | 434 | R | | | | | | | [symbol] |
| Q | 433 | R | | | | | | | | | | [symbol] |
| G | 439 | K | | | | | | | | | | [symbol] |
| H | 441 | K | | | | | | | | | | [symbol] |
| K | 442 | E | | | | | | | | | | [symbol] |
| K | 446 | R | D | 450 | E | | | | | | | [symbol] |
| D | 449 | E | | | | | | | | | | [symbol] |
| W | 450 | L | | | | | | | | | | [symbol] |
| E | 452 | K | | | | | | | | | | [symbol] |
| K | 453 | L | | | | | | | | | | [symbol] |
| R | 455 | E | | | | | | | | | | [symbol] |
| I | 456 | E | | | | | | | | | | [symbol] |
| I | 458 | T | | | | | | | | | | [symbol] |
| N | 461 | G | | | | | | | | | | [symbol] |
| N | 461 | G | S | 466 | Y | | | | | | | [symbol] |
| I | 468 | L | | | | | | | | | | [symbol] |
| M | 471 | L | | | | | | | | | | [symbol] |
| I | 475 | V | | | | | | | | | | [symbol] |
| T | 476 | L | | | | | | | | | | [symbol] |
| V | 477 | L | | | | | | | | | | [symbol] |

[symbol]

indicates data missing or illegible when filed

TABLE 12
Fold-change in activity over parental enzyme for point
mutants of MbUGT1-2 (1-2′ glycosylating activity).

| Wild- | | | | Wild- | | | | |
|---|---|---|---|---|---|---|---|---|
| type | | | | type | | | Fold increase | Fold increase |
| residue | Position | Mutation | Insertion | residue | Position | Mutation | steviolbioside | RebD |
| S | 14 | W | | | | | 1.8 | 2.4 |
| S | 14 | Y | | | | | 1.7 | 2.3 |
| V | 89 | A | | | | | | |
| G | 185 | A | | | | | 1.1 | 0.6 |
| V | 365 | I | | | | | 1.4 | 1.9 |
| E | 366 | P | | | | | 2.0 | 3.0 |
| V | 395 | Y | | F | 396 | T | 0.1 | 0.2 |
| G | 417 | T | | | | | 0.0 | 0.0 |
| H | 420 | E | | | | | 1.5 | 1.8 |
| M | 421 | F | | | | | 1.2 | 1.7 |
| M | 421 | I | | | | | 0.3 | 0.3 |
| M | 421 | A | | | | | 0.7 | 0.4 |
| S | 424 | D | | | | | 1.6 | 2.1 |
| S | 424 | Y | | | | | 0.8 | 0.4 |
| | | | GPS | | | | 0.9 | 1.5 |
| | | | between | | | | | |
| | | | 425 and 426 | | | | | |
| D | 427 | E | | | | | 1.2 | 1.5 |
| D | 427 | S | | | | | 1.1 | 1.0 |
| D | 427 | W | | | | | | |
| R | 428 | E | | | | | 1.4 | 1.9 |
| R | 428 | H | | | | | 1.0 | 1.1 |
| R | 428 | W | | | | | 0.8 | 0.5 |
| E | 431 | A | | | | | | |
| E | 431 | Y | | | | | 1.5 | 1.7 |
| R | 432 | H | | | | | 1.8 | 2.7 |
| R | 432 | W | | | | | 1.7 | 2.7 |
| K | 463 | D | | | | | 1.3 | 1.4 |
| K | 463 | E | | | | | 1.4 | 2.0 |

TABLE 13
Fold-change in activity over parental enzyme for point mutants of SrUGT76G1 (1-3′ glycosylating activity).

| | | | | | | | | | | | | | | | | Fold | Fold |
|---|---|---|---|---|---|---|---|---|---|---|---|---|---|---|---|---|---|
| | | | | | | | | | | | | | | | | increase | increase |
| WT | Pos. | Mut. | Insert. | Delet. | WT | Pos. | Mut. | Delet. | WT | Pos. | Mut. | Delet. | WT | Pos. | Mut. | RebA | RebM |
| F | 22 | V | | | | | | | | | | | | | | 0.3 | 0.1 |
| S | 77 | A | | | | | | | | | | | | | | 1.4 | 1.5 |
| N | 78 | A | | | | | | | | | | | | | | 1.1 | 1.2 |
| T | 81 | A | | | | | | | | | | | | | | 1.0 | 1.3 |
| H | 82 | A | | | | | | | | | | | | | | 1.4 | 0.7 |
| G | 87 | V | | | | | | | | | | | | | | 0.0 | 0.1 |
| | | | | G87- | | | | | | | | | | | | 0.1 | 1.0 |
| | | | | P91 | | | | | | | | | | | | | |
| I | 90 | V | | | | | | | | | | | | | | 1.1 | 0.4 |
| P | 91 | A | | | | | | | | | | | | | | 0.6 | 0.5 |
| I | 93 | V | | | | | | | | | | | | | | 1.0 | 0.3 |
| N | 94 | G | | | | | | | | | | | | | | 1.3 | 0.5 |
| L | 126 | G | | | | | | | | | | | | | | 0.5 | 0.4 |
| W | 127 | F | | | | | | | | | | | | | | 0.9 | 0.2 |
| M | 145 | V | | | | | | | | | | | | | | 0.5 | 0.4 |
| S | 147 | G | | | | | | | | | | | | | | 1.2 | 0.4 |
| N | 151 | A | | | | | | | | | | | | | | 0.7 | 0.4 |
| H | 155 | A | | | | | | | | | | | | | | 0.7 | 0.3 |
| Y | 155 | Y | | | | | | | | | | | | | | 1.4 | 0.2 |
| S | 192 | A | | | | | | | | | | | | | | 1.0 | 1.1 |
| Y | 194 | G | | | | | | | | | | | | | | 0.8 | 0.1 |
| | | | G | | | | | | | | | | | | | 2.4 | 1.1 |
| | | | between | | | | | | | | | | | | | | |
| | | | 194 and | | | | | | | | | | | | | | |
| | | | 195 | | | | | | | | | | | | | | |
| W | 197 | P | | | | | | | | | | | | | | 1.7 | 0.7 |
| I | 199 | A | | | | | | | | | | | | | | 0.9 | 0.1 |
| L | 200 | A | | | T | 284 | A | | | | | | | | | 8.2 | 8.6 |
| L | 200 | A | | | L | 379 | G | | T | 284 | A | | S | 192 | A | 7.0 | 9.0 |
| L | 200 | A | | | L | 379 | G | | T | 284 | A | | | | | 9.4 | 9.2 |
| L | 200 | A | | | L | 379 | G | | | | | G87- | | | | 0.5 | |
| | | | | | | | | | | | | P91 | | | | | |
| L | 200 | A | | | L | 379 | G | | | | | | | | | 3.1 | 1.8 |
| L | 200 | A | | | | | | | | | | | | | | 8.0 | 17.0 |
| L | 200 | G | | | | | | | | | | | | | | 6.7 | 3.9 |
| L | 200 | V | | | | | | | | | | | | | | 1.1 | 0.1 |
| L | 200 | A | | | | | | G87- | | | | | | | | 1.6 | 1.6 |
| | | | | | | | | P91 | | | | | | | | | |
| E | 202 | D | | | | | | | | | | | | | | 1.2 | 0.1 |
| I | 203 | A | | | | | | | | | | | | | | 1.2 | 0.2 |
| L | 204 | A | | | | | | | | | | | | | | 0.6 | 0.2 |
| G | 205 | R | | | K | 206 | A | | K | 209 | E | | | | | 0.8 | 0.1 |
| G | 205 | A | | | | | | | | | | | | | | 1.4 | 0.4 |
| M | 207 | A | | | | | | | | | | | | | | 0.8 | 0.6 |
| T | 284 | A | | | | | | | | | | | | | | 1.0 | 1.5 |
| T | 284 | V | | | | | | | | | | | | | | 1.6 | 0.3 |
| L | 379 | G | | | T | 284 | A | | | | | | | | | 1.1 | 1.5 |
| L | 379 | G | | | | | | | | | | | | | | 0.6 | 2.0 |
| L | 379 | A | | | | | | | | | | | | | | 1.9 | 1.4 |
| L | 397 | V | | | | | | | | | | | | | | 2.1 | 0.9 |

1-80. (canceled)

81. A method for making rebaudioside M comprising: culturing a host cell in the presence of rebaudioside D, the host cell expressing a uridine diphosphate-dependent glycosyltransferase (UGT) enzyme comprising an amino acid sequence that has at least 90% sequence identity to SEQ ID NO: 3 and has one or more amino acid modifications with respect to SEQ ID NO: 3 selected from: S77A, N78A, T81A, S192A, insertion of G after amino acid 194, L200A, L200G, T284A, L379G, and L379A.

82. The method of claim 81, wherein the UGT enzyme comprises an amino acid sequence having at least 95% sequence identity to SEQ ID NO: 3.

83. The method of claim 81, wherein the UGT enzyme comprises an amino acid having at least 97% sequence identity to SEQ ID NO: 3.

84. The method of claim 81, wherein the UGT enzyme comprises an amino acid sequence having at least 98% sequence identity to SEQ ID NO: 3.

85. The method of claim 81, wherein the UGT enzyme comprises an amino acid sequence having at least 99% sequence identity to SEQ ID NO: 3.

86. The method of claim 81, wherein the UGT enzyme comprises an amino acid sequence having an amino acid modification corresponding to L200A or L200G, and optionally T284A, with respect to SEQ ID NO: 3.

87. The method of claim 81, wherein the UGT enzyme comprises an Ala inserted or substituted at position 2, with respect to SEQ ID NO: 3.

88. The method of claim 81, wherein the host cell further expresses a UGT enzyme comprising an amino acid sequence having at least 90% sequence identity to an amino acid sequence selected from: SEQ ID NO: 7, SEQ ID NO: 5, SEQ ID NO: 4, SEQ ID NO: 6, and SEQ ID NO: 45.

89. The method of claim 88, wherein the host cell further expresses a UGT enzyme comprising an amino acid sequence having at least 90% sequence identity to SEQ ID NO: 1, and a UGT enzyme comprising an amino acid sequence having at least 90% sequence identity to SEQ ID NO: 2.

90. The method of claim 81, wherein the host cell is prokaryotic.

91. The method of claim 90, wherein the host cell is a bacterial cell selected from E. coli, Bacillus subtillus, or Pseudomonas putida.

92. The method of claim 81, wherein the host cell is Saccharomyces cerevisiae, Pichia pastoris, or Yarrowia lipolytica.

93. The method of claim 81, wherein the host cell further comprises one or more genetic modifications increasing the availability of UDP-glucose.

94. The method of claim 93, wherein the host cell is E. coli comprising one or more genetic modifications selected from ΔgalE, ΔgalT, ΔgalK, ΔgalM, ΔushA, Δagp, Δpgm, duplication of E coli GALU, and expression of Bacillus substillus UGPA, BaSP.


SEQUENCE LISTING (65 sequences)

SEQ ID NO: 1 - protein, 481 aa, Stevia rebaudiana
MDAMATTEKKPHVIFIPFPAQSHIKAMLKLAQLLHHKGLQITFVNTDFIHNQFLESSGPHCLDGAPGFRFETIPDGVSHSPEASIPIRESLLRSIETNFLDRFIDLVTKLPDPPTCIISDGFLSVFTIDAAKKLGIPVMMYWTLAACGFMGFYHIHSLIEKGFAPLKDASYLTNGYLDTVIDWVPGMEGIRLKDFPLDWSTDLNDKVLMFTTEAPQRSHKVSHHIFHTFDELEPSIIKTLSLRYNHIYTIGPLQLLLDQIPEEKKQTGITSLHGYSLVKEEPECFQWLQSKEPNSVVYVNFGSTTVMSLEDMTEFGWGLANSNHYFLWIIRSNLVIGENAVLPPELEEHIKKRGFIASWCSQEKVLKHPSVGGFLTHCGWGSTIESLSAGVPMICWPYSWDQLTNCRYICKEWEVGLEMGTKVKRDEVKRLVQELMGEGGHKMRNKAKDWKEKARIAIAPNGSSSLNIDKMVKEITVLARN

SEQ ID NO: 2 - protein, 460 aa, Stevia rebaudiana
MAEQQKIKKSPHVLLIPFPLQGHINPFIQFGKRLISKGVKTTLVTTIHTLNSTLNHSNTTTTSIEIQAISDGCDEGGFMSAGESYLETFKQVGSKSLADLIKKLQSEGTTIDAIIYDSMTEWVLDVAIEFGIDGGSFFTQACVVNSLYYHVHKGLISLPLGETVSVPGFPVLQRWETPLILQNHEQIQSPWSQMLFGQFANIDQARWVFTNSFYKLEEEVIEWTRKIWNLKVIGPTLPSMYLDKRLDDDKDNGFNLYKANHHECMNWLDDKPKESVVYVAFGSLVKHGPEQVEEITRALIDSDVNFLWVIKHKEEGKLPENLSEVIKTGKGLIVAWCKQLDVLAHESVGCFVTHCGFNSTLEAISLGVPVVAMPQFSDQTTNAKLLDEILGVGVRVKADENGIVRRGNLASCIKMIMEEERGVIIRKNAVKWKDLAKVAVHEGGSSDNDIVEFVSELIKA

SEQ ID NO: 3 - protein, 458 aa, Stevia rebaudiana
MENKTETTVRRRRRIILFPVPFQGHINPILQLANVLYSKGFSITIFHTNFNKPKTSNYPHFTFRFILDNDPQDERISNLPTHGPLAGMRIPIINEHGADELRRELELLMLASEEDEEVSCLITDALWYFAQSVADSLNLRRLVLMTSSLFNFHAHVSLPQFDELGYLDPDDKTRLEEQASGFPMLKVKDIKSAYSNWQILKEILGKMIKQTKASSGVIWNSFKELEESELETVIREIPAPSFLIPLPKHLTASSSSLLDHDRTVFQWLDQQPPSSVLYVSFGSTSEVDEKDFLEIARGLVDSKQSFLWVVRPGFVKGSTWVEPLPDGFLGERGRIVKWVPQQEVLAHGAIGAFWTHSGWNSTLESVCEGVPMIFSDFGLDQPLNARYMSDVLKVGVYLENGWERGEIANAIRRVMVDEEGEYIRQNARVLKQKADVSLMKGGSSYESLESLVSYISSL

SEQ ID NO: 4 - protein, 485 aa, Stevia rebaudiana
MYNVTYHQNSKAMATSDSIVDDRKQLHVATFPWLAFGHILPFLQLSKLIAEKGHKVSFLSTTRNIQRLSSHISPLINVVQLTLPRVQELPEDAEATTDVHPEDIQYLKKAVDGLQPEVTRFLEQHSPDWIIYDFTHYWLPSIAASLGISRAYFCVITPWTIAYLAPSSDAMINDSDGRTTVEDLTTPPKWFPFPTKVCWRKHDLARMEPYEAPGISDGYRMGMVFKGSDCLLFKCYHEFGTQWLPLLETLHQVPVVPVGLLPPEIPGDEKDETWVSIKKWLDGKQKGSVVYVALGSEALVSQTEVVELALGLELSGLPFVWAYRKPKGPAKSDSVELPDGFVERTRDRGLVWTSWAPQLRILSHESVCGFLTHCGSGSIVEGLMFGHPLIMLPIFCDQPLNARLLEDKQVGIEIPRNEEDGCLTKESVARSLRSVVVENEGEIYKANARALSKIYNDTKVEKEYVSQFVDYLEKNARAVAIDHES

SEQ ID NO: 5 - protein, 473 aa, Stevia rebaudiana
MATSDSIVDDRKQLHVATFPWLAFGHILPYLQLSKLIAEKGHKVSFLSTTRNIQRLSSHISPLINVVQLTLPRVQELPEDAEATTDVHPEDIPYLKKASDGLQPEVTRFLEQHSPDWIIYDYTHYWLPSIAASLGISRAHFSVTTPWAIAYMGPSADAMINGSDGRTTVEDLTTPPKWFPFPTKVCWRKHDLARLVPYKAPGISDGYRMGLVLKGSDCLLSKCYHEFGTQWLPLLETLHQVPVVPVGLLPPEVPGDEKDETWVSIKKWLDGKQKGSVVYVALGSEVLVSQTEVVELALGLELSGLPFVWAYRKPKGPAKSDSVELPDGFVERTRDRGLVWTSWAPQLRILSHESVCGFLTHCGSGSIVEGLMFGHPLIMLPIFGDQPLNARLLEDKQVGIEIPRNEEDGCLTKESVARSLRSVVVEKEGEIYKANARELSKIYNDTKVEKEYVSQFVDYLEKNTRAVAIDHES

SEQ ID NO: 6 - protein, 473 aa, Stevia rebaudiana
MATSDSIVDDRKQLHVATFPWLAFGHILPYLQLSKLIAEKGHKVSFLSTTRNIQRLSSHISPLINVVQLTLPRVQELPEDAEATTDVHPEDIPYLKKASDGLQPEVTRFLEQHSPDWIIYDYTHYWLPSIAASLGISRAHFSVTTPWAIAYMGPSADAMINGSDGRTTVEDLTTPPKWFPFPTKVCWRKHDLARLVPYKAPGISDGYRMGLVLKGSDCLLSKCYHEFGTQWLPLLETLHQVPVVPVGLLPPEIPGDEKDETWVSIKKWLDGKQKGSVVYVALGSEVLVSQTEVVELALGLELSGLPFVWAYRKPKGPAKSDSVELPDGFVERTRDRGLVWTSWAPQLRILSHESVCGFLTHCGSGSIVEGLMFGHPLIMLPIFGDQPLNARLLEDKQVGIEIPRNEEDGCLTKESVARSLRSVVVEKEGEIYKANARELSKIYNDTKVEKEYVSQFVDYLEKNARAVAIDHES

SEQ ID NO: 7 - protein, 462 aa, Oryza sativa
MDSGYSSSYAAAAGMHVVICPWLAFGHLLPCLDLAQRLASRGHRVSFVSTPRNISRLPPVRPALAPLVAFVALPLPRVEGLPDGAESTNDVPHDRPDMVELHRRAFDGLAAPFSEFLGTACADWVIVDVFHHWAAAAALEHKVPCAMMLLGSAHMIASIADRRLERAETESPAAAGQGRPAAAPTFEVARMKLIRTKGSSGMSLAERFSLTLSRSSLVVGRSCVEFEPETVPLLSTLRGKPITFLGLMPPLHEGRREDGEDATVRWLDAQPAKSVVYVALGSEVPLGVEKVHELALGLELAGTRFLWALRKPTGVSDADLLPAGFEERTRGRGVVATRWVPQMSILAHAAVGAFLTHCGWNSTIEGLMFGHPLIMLPIFGDQGPNARLIEAKNAGLQVARNDGDGSFDREGVAAAIRAVAVEEESSKVFQAKAKKLQEIVADMACHERYIDGFIQQLRSYKD

SEQ ID NO: 8 - protein, 463 aa, synthetic construct
MAECMNWLDDKPKESVVYVAFGSLVKHGPEQVEEITRALIDSDVNFLWVIKHKEEGKLPENLSEVIKTGKGLIVAWCKQLDVLAHESVGCFVTHCGFNSTLEAISLGVPVVAMPQFSDQTTNAKLLDEILGVGVRVKADENGIVRRGNLASCIKMIMEEERGVIIRKNAVKWKDLAKVAVHEGGSSDNDIVEFVSELIKAGSGEQQKIKKSPHVLLIPFPLQGHINPFIQFGKRLISKGVKTTLVTTIHTLNSTLNHSNTTTTSIEIQAISDGCDEGGFMSAGESYLETFKQVGSKSLADLIKKLQSEGTTIDAIIYDSMTEWVLDVAIEFGIDGGSFFTQACVVNSLYYHVHKGLISLPLGETVSVPGFPVLQRWETPLILQNHEQIQSPWSQMLFGQFANIDQARWVFTNSFYKLEEEVIEWTRKIWNLKVIGPTLPSMYLDKRLDDDKDNGFNLYKANHH

SEQ ID NO: 9 - protein, 463 aa, synthetic construct
MAGSSGMSLAERFSLTLSRSSLVVGRSCVEFEPETVPLLSTLRGKPITFLGLMPPLHEGRREDGEDATVRWLDAQPAKSVVYVALGSEVPLGVEKVHELALGLELAGTRFLWALRKPTGVSDADLLPAGFEERTRGRGVVATRWVPQMSILAHAAVGAFLTHCGWNSTIEGLMFGHPLIMLPIFGDQGPNARLIEAKNAGLQVARNDGDGSFDREGVAAAIRAVAVEEESSKVFQAKAKKLQEIVADMACHERYIDGFIQQLRSYKDDSGYSSSYAAAAGMHVVICPWLAFGHLLPCLDLAQRLASRGHRVSFVSTPRNISRLPPVRPALAPLVAFVALPLPRVEGLPDGAESTNDVPHDRPDMVELHRRAFDGLAAPFSEFLGTACADWVIVDVFHHWAAAAALEHKVPCAMMLLGSAHMIASIADRRLERAETESPAAAGQGRPAAAPTFEVARMKLIRTK

SEQ ID NO: 10 - protein, 459 aa, synthetic construct
MANWQILKEILGKMIKQTKASSGVIWNSFKELEESELETVIREIPAPSFLIPLPKHLTASSSSLLDHDRTVFQWLDQQPPSSVLYVSFGSTSEVDEKDFLEIARGLVDSKQSFLWVVRPGFVKGSTWVEPLPDGFLGERGRIVKWVPQQEVLAHGAIGAFWTHSGWNSTLESVCEGVPMIFSDFGLDQPLNARYMSDVLKVGVYLENGWERGEIANAIRRVMVDEEGEYIRQNARVLKQKADVSLMKGGSSYESLESLVSYISSLENKTETTVRRRRRIILFPVPFQGHINPILQLANVLYSKGFSITIFHTNFNKPKTSNYPHFTFRFILDNDPQDERISNLPTHGPLAGMRIPIINEHGADELRRELELLMLASEEDEEVSCLITDALWYFAQSVADSLNLRRLVLMTSSLFNFHAHVSLPQFDELGYLDPDDKTRLEEQASGFPMLKVKDIKSAYS

SEQ ID NO: 11 - protein, 392 aa, Taxus canadensis
MYTAMAAGTQSLQLRTVASYQECNSMRSCFKLTPFKSFHGVNFNVPSLGAANCEIMGHLKLGSLPYKQCSVSSKSTKTMAQLVDLAETEKAEGKDIEFDFNEYMKSKAVAVDAALDKAIPLEYPEKIHESMRYSLLAGGKRVRPALCIAACELVGGSQDLAMPTACAMEMIHTMSLIHDDLPCMDNDDFRRGKPTNHKVFGEDTAVLAGDALLSFAFEHIAVATSKTVPSDRTLRVISELGKTIGSQGLVGGQVVDITSEGDANVDLKTLEWIHIHKTAVLLECSVVSGGILGGATEDEIARIRRYARCVGLLFQVVDDILDVTKSSEELGKTAGKDLLTDKATYPKLMGLEKAKEFAAELATRAKEELSSFDQIKAAPLLGLADYIAFRQN

SEQ ID NO: 12 - protein, 296 aa, Taxus canadensis
MADFNEYMKSKAVAVDAALDKAIPLEYPEKIHESMRYSLLAGGKRVRPALCIAACELVGGSQDLAMPTACAMEMIHTMSLIHDDLPCMDNDDFRRGKPTNHKVFGEDTAVLAGDALLSFAFEHIAVATSKTVPSDRTLRVICELGKTIGSQGLVGGQVVDITSEGDANVDLKTLEWIHIHKTAVLLECSVVSGGILGDATEDEIARIRRYARCVGLLFQVVDDILDVTKSSEELGKTAGKDLLTDKATYPKLMGLEKAKEFAAELATRAKEELSSFDQIKVAPLLGLADYIAFRQN

SEQ ID NO: 13 - protein, 383 aa, Abies grandis
MAYSGMATSYHGLHFMNIATQECNLKRLSIPSRRFHGVSPSLWASNGFQGHLKRELSANSFLVSSSRYSNTIAKFTNLPEKVKEKVIEFDFKEYLRSKAMAVNEALDRAVPLRYPERIHEAMRYSLLAGGKRVRPVLCISACELVGGTEEVAMPTACAMEMIHTMSLIHDDLPCMDNDDFRRGKPTNHKVFGEGTAILAGDALLSFAFEHIAVSTSKSVGTDRILRVVSELGRTIGSQGLVGGQVADITSEGDASVDLDTLEWIHIHKTAVLLECSVMCGAIISGASDNEIERIQRYARSVGLLFQVVDDILDVTKSSKELGKTAGKDLISDKATYPKLMGLEKAKQFASDLLIRAKEDLSCFDPMKAAPLLGLADYIAFRQN

SEQ ID NO: 14 - protein, 397 aa, synthetic construct
MSPPLDSALEPLSEYKETAFPRTEKDPSQYKEHDLVTPEKEIQTGYFSPRGSHSSHGSHDSSASSNISLDDARMSDVNNSPNVFHDDPDTIDEKLSMYWKAANETVIREPYDYIAGIPGKEIRRKLLEAFNHWYKVDEQSCQAIATTVGMAHNASLLIDDIQDSSKLRRGVPCAHEVFGIAQTINSANYVYFLAQNQLFRLRSWPQAISVFNEEMVNLHRGQGMELFWRDNLLPPSMDDYLQMIANKTGGLFRMIVRLLQTSSRQVIDVEQLVDVLGLYFQILDDYKNIREEKMAAQKGFFEDLTEGKFSFPICHAIGEGAKNRTALLHMLRLKTDDMKIKQEAVCILDNAGSLDYTREVLYGLDRKARSLLREFKTPNPFMEALLDAMLSSLQACH

SEQ ID NO: 15 - protein, 359 aa, Streptomyces melanosporofaciens
MTTPTLSPGRLDADTVRKSVDVVLEDFLTAKAHTTPQHHLPYLSGLLKDFLSGGKRIRPLLCVTGWQAVGGGEDTEPVFRVAACLEMFHAFALIHDDVMDDSDTRRGRPTIHRTLAALCATDRRPEQIERFGVSGAVLLGDLALTWSDELLHSAGLTPVQFDAVLPLLSEMRTEVMLGQYLDLQATGELTDDVEATLTVNRYKTAKYTIERPLHVGAAIAGAGPEAMEAFTAYALPLGEAFQLRDDLLGVYGDPESTGKSQLDDLRAGKNTTLIALALRGSDSTQAARLRSLIGNPLLDERDAATIQEIFAATTARDAVEQMIDDRRTQALRALDDAPFTADAVNALKQIARLATVRNS

SEQ ID NO: 16 - protein, 787 aa, Stevia rebaudiana
MKTGFISPATVFHHRISPATTFRHHLSPATTNSTGIVALRDINFRCKAVSKEYSDLLQKDEASFTKWDDDKVKDHLDTNKNLYPNDEIKEFVESVKAMFGSMNDGEINVSAYDTAWVALVQDVDGSGSPQFPSSLEWIANNQLSDGSWGDHLLFSAHDRIINTLACVIALTSWNVHPSKCEKGLNFLRENICKLEDENAEHMPIGFEVTFPSLIDIAKKLNIEVPEDTPALKEIYARRDIKLTKIPMEVLHKVPTTLLHSLEGMPDLEWEKLLKLQCKDGSFLFSPSSTAFALMQTKDEKCLQYLTNIVTKFNGGVPNVYPVDLFEHIWVVDRLQRLGIARYFKSEIKDCVEYINKYWTKNGICWARNTHVQDIDDTAMGFRVLRAHGYDVTPDVFRQFEKDGKFVCFAGQSTQAVTGMFNVYRASQMLFPGERILEDAKKFSYNYLKEKQSTNELLDKWIIAKDLPGEVGYALDIPWYASLPRLETRYYLEQYGGEDDVWIGKTLYRMGYVSNNTYLEMAKLDYNNYVAVLQLEWYTIQQWYVDIGIEKFESDNIKSVLVSYYLAAASIFEPERSKERIAWAKTTILVDKITSIFDSSQSSKEDITAFIDKFRNKSSSKKHSINGEPWHEVMVALKKTLHGFALDALMTHSQDIHPQLHQAWEMWLTKLQDGVDVTAELMVQMINMTAGRWVSKELLTHPQYQRLSTVTNSVCHDITKLHNFKENSTTVDSKVQELVQLVFSDTPDDLDQDMKQTFLTVMKTFYYKAWCDPNTINDHISKVFEIVI

SEQ ID NO: 17 - protein, 784 aa, Stevia rebaudiana
MNLSLCIASPLLTKSNRPAALSAIHTASTSHGGQTNPTNLIIDTTKERIQKQFKNVEISVSSYDTAWVAMVPSPNSPKSPCFPECLNWLINNQLNDGSWGLVNHTHNHNHPLLKDSLSSTLACIVALKRWNVGEDQINKGLSFIESNLASATEKSQPSPIGFDIIFPGLLEYAKNLDINLLSKQTDFSLMLHKRELEQKRCHSNEMDGYLAYISEGLGNLYDWNMVKKYQMKNGSVFNSPSATAAAFINHQNPGCLNYLNSLLDKFGNAVPTVYPHDLFIRLSMVDTIERLGISHHFRVEIKNVLDETYRCWVERDEQIFMDVVTCALAFRLLRINGYEVSPDPLAEITNELALKDEYAALETYHASHILYQEDLSSGKQILKSADFLKEIISTDSNRLSKLIHKEVENALKFPINTGLERINTRRNIQLYNVDNTRILKTTYHSSNISNTDYLRLAVEDFYTCQSIYREELKGLERWVVENKLDQLKFARQKTAYCYFSVAATLSSPELSDARISWAKNGILTTVVDDFFDIGGTIDELTNLIQCVEKWNVDVDKDCCSEHVRILFLALKDAICWIGDEAFKWQARDVTSHVIQTWLELMNSMLREAIWTRDAYVPTLNEYMENAYVSFALGPIVKPAIYFVGPKLSEEIVESSEYHNLFKLMSTQGRLLNDIHSFKREFKEGKLNAVALHLSNGESGKVEEEVVEEMMMMIKNKRKELMKLIFEENGSIVPRACKDAFWNMCHVLNFFYANDDGFTGNTILDTVKDIIYNPLVLVNENEEQR

SEQ ID NO: 18 - protein, 952 aa, synthetic construct
MPGKIENGTPKDLKTGNDFVSAAKSLLDRAFKSHHSYYGLCSTSCQVYDTAWVAMIPKTRDNVKQWLFPECFHYLLKTQAADGSWGSLPTTQTAGILDTASAVLALLCHAQEPLQILDVSPDEMGLRIEHGVTSLKRQLAVWNDVEDTNHIGVEFIIPALLSMLEKELDVPSFEFPCRSILERMHGEKLGHFDLEQVYGKPSSLLHSLEAFLGKLDFDRLSHHLYHGSMMASPSSTAAYLIGATKWDDEAEDYLRHVMRNGAGHGNGGISGTFPTTHFECSWIIATLLKVGFTLKQIDGDGLRGLSTILLEALRDENGVIGFAPRTADVDDTAKALLALSLVNQPVSPDIMIKVFEGKDHFTTFGSERDPSLTSNLHVLLSLLKQSNLSQYHPQILKTTLFTCRWWWGSDHCVKDKWNLSHLYPTMLLVEAFTEVLHLIDGGELSSLFDESFKCKIGLSIFQAVLRIILTQDNDGSWRGYREQTCYAILALVQARHVCFFTHMVDRLQSCVDRGFSWLKSCSFHSQDLTWTSKTAYEVGFVAEAYKLAALQSASLEVPAATIGHSVTSAVPSSDLEKYMRLVRKTALFSPLDEWGLMASIIESSFFVPLLQAQRVEIYPRDNIKVDEDKYLSIIPFTWVGCNNRSRTFASNRWLYDMMYLSLLGYQTDEYMEAVAGPVFGDVSLLHQTIDKVIDNTMGNLARANGTVHSGNGHQHESPNIGQVEDTLTRFTNSVLNHKDVLNSSSSDQDTLRREFRTFMHAHITQIEDNSRFSKQASSDAFSSPEQSYFQWVNSTGGSHVACAYSFAFSNCLMSANLLQGKDAFPSGTQKYLISSVMRHATNMCRMYNDFGSIARDNAERNVNSIHFPEFTLCNGTSQNLDERKERLLKIATYEQGYLDRALEALERQSRDDAGDRAGSKDMRKLKIVKLFCDVTDLYDQLYVIKDLSSSMK

SEQ ID NO: 19 - protein, 881 aa, Physcomitrella patens
MASSTLIQNRSCGVTSSMSSFQIFRGQPLRFPGTRTPAAVQCLKKRRCLRPTESVLESSPGSGSYRIVTGPSGINPSSNGHLQEGSLTHRLPIPMEKSIDNFQSTLYVSDIWSETLQRTECLLQVTENVQMNEWIEEIRMYFRNMTLGEISMSPYDTAWVARVPALDGSHGPQFHRSLQWIIDNQLPDGDWGEPSLFLGYDRVCNTLACVIALKTWGVGAQNVERGIQFLQSNIYKMEEDDANHMPIGFEIVFPAMMEDAKALGLDLPYDATILQQISAEREKKMKKIPMAMVYKYPTTLLHSLEGLHREVDWNKLLQLQSENGSFLYSPASTACALMYTKDVKCFDYLNQLLIKFDHACPNVYPVDLFERLWMVDRLQRLGISRYFEREIRDCLQYVYRYWKDCGIGWASNSSVQDVDDTAMAFRLLRTHGFDVKEDCFRQFFKDGEFFCFAGQSSQAVTGMFNLSRASQTLFPGESLLKKARTFSRNFLRTKHENNECFDKWIITKDLAGEVEYNLTFPWYASLPRLEHRTYLDQYGIDDIWIGKSLYKMPAVTNEVFLKLAKADFNMCQALHKKELEQVIKWNASCQFRDLEFARQKSVECYFAGAATMFEPEMVQARLVWARCCVLTTVLDDYFDHGTPVEELRVFVQAVRTWNPELINGLPEQAKILFMGLYKTVNTIAEEAFMAQKRDVHHHLKHYWDKLITSALKEAEWAESGYVPTFDEYMEVAEISVALEPIVCSTLFFAGHRLDEDVLDSYDYHLVMHLVNRVGRILNDIQGMKREASQGKISSVQIYMEEHPSVPSEAMAIAHLQELVDNSMQQLTYEVLRFTAVPKSCKRIHLNMAKIMHAFYKDTDGFSSLTAMTGFVKKVLFEPVPE

SEQ ID NO: 20 - protein, 946 aa, Phaeosphaeria sp.
MFAKFDMLEEEARALVRKVGNAVDPIYGFSTTSCQIYDTAWAAMISKEEHGDKVWLFPESFKYLLEKQGEDGSWERHPRSKTVGVLNTAAACLALLRHVKNPLQLQDIAAQDIELRIQRGLRSLEEQLIAWDDVLDTNHIGVEMIVPALLDYLQAEDENVDFEFESHSLLMQMYKEKMARFSPESLYRARPSSALHNLEALIGKLDFDKVGHHLYNGSMMASPSSTAAFLMHASPWSHEAEAYLRHVFEAGTGKGSGGFPGTYPTTYFELNWVLSTLMKSGFTLSDLECDELSSIANTIAEGFECDHGVIGFAPRAVDVDDTAKGLLTLTLLGMDEGVSPAPMIAMFEAKDHFLTFLGERDPSFTSNCHVLLSLLHRTDLLQYLPQIRKTTTFLCEAWWACDGQIKDKWHLSHLYPTMLMVQAFAEILLKSAEGEPLHDAFDAATLSRVSICVFQACLRTLLAQSQDGSWHGQPEASCYAVLTLAESGRLVLLQALQPQIAAAMEKAADVMQAGRWSCSDHDCDWTSKTAYRVDLVAAAYRLAAMKASSNLTFTVDDNVSKRSNGFQQLVGRTDLFSGVPAWELQASFLESALFVPLLRNHRLDVFDRDDIKVSKDHYLDMIPFTWVGCNNRSRTYVSTSFLFDMMIISMLGYQIDEFFEAEAAPAFAQCIGQLHQVVDKVVDEVIDEVVDKVVGKVVGKVVGKVVDERVDSPTHEAIAICNIEASLRRFVDHVLHHQHVLHASQQEQDILWRELRAFLHAHVVQMADNSTLAPPGRTFFDWVRTTAADHVACAYSFAFACCITSATIGQGQSMFATVNELYLVQAAARHMTTMCRMCNDIGSVDRDFIEANINSVHFPEFSTLSLVADKKKALARLAAYEKSCLTHTLDQFENEVLQSPRVSSAASGDFRTRKVAVVRFFADVTDFYDQLYILRDLSSSLKHVGT

SEQ ID NO: 21 - protein, 513 aa, Stevia rebaudiana
MDAVTGLLTVPATAITIGGTAVALAVALIFWYLKSYTSARRSQSNHLPRVPEVPGVPLLGNLLQLKEKKPYMTFTRWAATYGPIYSIKTGATSMVVVSSNEIAKEALVTRFQSISTRNLSKALKVLTADKTMVAMSDYDDYHKTVKRHILTAVLGPNAQKKHRIHRDIMMDNISTQLHEFVKNNPEQEEVDLRKIFQSELFGLAMRQALGKDVESLYVEDLKITMNRDEIFQVLVVDPMMGAIDVDWRDFFPYLKWVPNKKFENTIQQMYIRREAVMKSLIKEHKKRIASGEKLNSYIDYLLSEAQTLTDQQLLMSLWEPIIESSDTTMVTTEWAMYELAKNPKLQDRLYRDIKSVCGSEKITEEHLSQLPYITAIFHETLRRHSPVPIIPLRHVHEDTVLGGYHVPAGTELAVNIYGCNMDKNVWENPEEWNPERFMKENETIDFQKTMAFGGGKRVCAGSLQALLTASIGIGRMVQEFEWKLKDMTQEEVNTIGLTTQMLRPLRAIIKPRI

SEQ ID NO: 22 - protein, 501 aa, synthetic construct [sequence omitted]

SEQ ID NO: 23 - protein, 509 aa, Arabidopsis thaliana
MAFFSMISILLGFVISSFIFIFFFKKLLSFSRKNMSEVSTLPSVPVVPGFPVIGNLLQLKEKKPHKTFTRWSEIYGPIYSIKMGSSSLIVLNSTETAKEAMVTRFSSISTRKLSNALTVLTCDKSMVATSDYDDFHKLVKRCLLNGLLGANAQKRKRHYRDALIENVSSKLHAHARDHPQEPVNFRAIFEHELFGVALKQAFGKDVESIYVKELGVTLSKDEIFKVLVHDMMEGAIDVDWRDFFPYLKWIPNKSFEARIQQKHKRRLAVMNALIQDRLKQNGSESDDDCYLNFLMSEAKTLTKEQIAILVWETIIETADTTLVTTEWAIYELAKHPSVQDRLCKEIQNVCGGEKFKEEQLSQVPYLNGVFHETLRKYSPAPLVPIRYAHEDTQIGGYHVPAGSEIAINIYGCNMDKKRWERPEDWWPERFLDDGKYETSDLHKTMAFGAGKRVCAGALQASLMAGIAIGRLVQEFEWKLRDGEEENVDTYGLTSQKLYPLMAIINPRRS

SEQ ID NO: 24 - protein, 546 aa, Physcomitrella patens
MAKHLATQLLQQWNEALKTMPPGFRTAGKILVWEELASNKVLITIALAWVLLFVARTCLRNKKRLPPAIPGGLPVLGNLLQLTEKKPHRTFTAWSKEHGPIFTIKVGSVPQAVVNNSEIAKEVLVTKFASISKRQMPMALRVLTRDKTMVAMSDYGEEHRMLKKLVMTNLLGPTTQNKNRSLRDDALIGMIEGVLAELKASPTSPKVVNVRDYVQRSLFPFALQQVFGYIPDQVEVLELGTCVSTWDMFDALVVAPLSAVINVDWRDFFPALRWIPNRSVEDLVRTVDFKRNSIMKALIRAQRMRLANLKEPPRCYADIALTEATHLTEKQLEMSLWEPIIESADTTLVTSEWAMYEIAKNPDCQDRLYREIVSVAGTERMVTEDDLPNMPYLGAIIKETLRKYTPVPLIPSRFVEEDITLGGYDIPKGYQILVNLFAIANDPAVWSNPEKWDPERMLANKKVDMGFRDFSLMPFGAGKRMCAGITQAMFIIPMNVAALVQHCEWRLSPQEISNINNKIEDVVYLTTHKLSPLSCEATPRISHRLP

SEQ ID NO: 25 - protein, 476 aa, Stevia rebaudiana
MIQVLTPILLFLIFFVFWKVYKHQKTKINLPPGSFGWPFLGETLALLRAGWDSEPERFVRERIKKHGSPLVFKTSLFGDRFAVLCGPAGNKFLFCNENKLVASWWPVPVRKLFGKSLLTIRGDEAKWMRKMLLSYLGPDAFATHYAVTMDVVTRRHIDVHWRGKEEVNVFQTVKLYAFELACRLFMNLDDPNHIAKLGSLFNIFLKGIIELPIDVPGTRFYSSKKAAAAIRIELKKLIKARKLELKEGKASSSQDLLSHLLTSPDENGMFLTEEEIVDNILLLLFAGHDTSALSITLLMKTLGEHSDVYDKVLKEQLEISKTKEAWESLKWEDIQKMKYSWSVICEVMRLNPPVIGTYREALVDIDYAGYTIPKGWKLHWSAVSTQRDEANFEDVTRFDPSRFEGAGPTPFTFVPFGGGPRMCLGKEFARLEVLAFLHNIVTNFKWDLLIPDEKIEYDPMATPAKGLPIRLHPHQV

SEQ ID NO: 26 - protein, 522 aa, Stevia rebaudiana
MGLFPLEDSYALVFEGLAITLALYYLLSFIYKTSKKTCTPPKASGEHPITGHLNLLSGSSGLPHLALASLADRCGPIFTIRLGIRRVLVVSNWEIAKEIFTTHDLIVSNRPKYLAAKILGFNYVSFSFAPYGPYWVGIRKIIATKLMSSSRLQKLQFVRVFELENSMKSIRESWKEKKDEEGKVLVEMKKWFWELNMNIVLRTVAGKQYTGTVDDADAKRISELFREWFHYTGRFVVGDAFPFLGWLDLGGYKKTMELVASRLDSMVSKWLDEHRKKQANDDKKEDMDFMDIMISMTEANSPLEGYGTDTIIKTTCMTLIVSGVDTTSIVLTWALSLLLNNRDTLKKAQEELDMCVGKGRQVNESDLVNLIYLEAVLKEALRLYPAAFLGGPRAFLEDCTVAGYRIPKGTCLLINMWKLHRDPNIWSDPCEFKPERFLTPNQKDVDVIGMDFELIPFGAGRRYCPGTRLALQMLHIVLATLLQNFEMSTPNDAPVDMTASVGMTNAKASPLEVLLSPRVKWS

SEQ ID NO: 27 - protein, 525 aa, Arabidopsis thaliana
MESLVVHTVNAIWCIVIVGIFSVGYHVYGRAVVEQWRMRRSLKLQGVKGPPPSIFNGNVSEMQRIQSEAKHCSGDNIISHDYSSSLFPHFDHWRKQYGRIYTYSTGLKQHLYINHPEMVKELSQTNTLNLGRITHITKRLNPILGNGIITSNGPHWAHQRRIIAYEFTHDKIKGMVGLMVESAMPMLNKWEEMVKRGGEMGCDIRVDEDLKDVSADVIAKACFGSSFSKGKAIFSMIRDLLTAITKRSVLFRFNGFTDMVFGSKKHGDVDIDALEMELESSIWETVKEREIECKDTHKKDLMQLILEGAMRSCDGNLWDKSAYRRFVVDNCKSIYFAGHDSTAVSVSWCLMLLALNPSWQVKIRDEILSSCKNGIPDAESIPNLKTVTMVIQETMRLYPPAPIVGREASKDIRLGDLVVPKGVCIWTLIPALHRDPEIWGPDANDFKPERFSEGISKACKYPQSYIPFGLGPRTCVGKNFGMMEVKVLVSLIVSKFSFTLSPTYQHSPSHKLLVEPQHGVVIRVV

SEQ ID NO: 28 - protein, 507 aa, synthetic construct
MALLLAVFVYGRAVVEQWRMRRSLKLQGVKGPPPSIFNGNVSEMQRIQSEAKHCSGDNIISHDYSSSLFPHFDHWRKQYGRIYTYSTGLKQHLYINHPEMVKELSQTNTLNLGRITHITKRLNPILGNGIITSNGPHWAHQRRIIAYEFTHDKIKGMVGLMVESAMPMLNKWEEMVKRGGEMGCDIRVDEDLKDVSADVIAKACFGSSFSKGKAIFSMIRDLLTAITKRSVLFRFNGFTDMVFGSKKHGDVDIDALEMELESSIWETVKEREIECKDTHKKDLMQLILEGAMRSCDGNLWDKSAYRRFVVDNCKSIYFAGHDSTAVSVSWCLMLLALNPSWQVKIRDEILSSCKNGIPDAESIPNLKTVTMVIQETMRLYPPAPIVGREASKDIRLGDLVVPKGVCIWTLIPALHRDPEIWGPDANDFKPERFSEGISKACKYPQSYIPFGLGPRTCVGKNFGMMEVKVLVSLIVSKFSFTLSPTYQHSPSHKLLVEPQHGVVIRVV

SEQ ID NO: 29 - protein, 519 aa, synthetic construct
MALLLAVFIVIVGIFSVGYHVYGRAVVEQWRMRRSLKLQGVKGPPPSIFNGNVSEMQRIQSEAKHCSGDNIISHDYSSSLFPHFDHWRKQYGRIYTYSTGLKQHLYINHPEMVKELSQTNTLNLGRITHITKRLNPILGNGIITSNGPHWAHQRRIIAYEFTHDKIKGMVGLMVESAMPMLNKWEEMVKRGGEMGCDIRVDEDLKDVSADVIAKACFGSSFSKGKAIFSMIRDLLTAITKRSVLFRFNGFTDMVFGSKKHGDVDIDALEMELESSIWETVKEREIECKDTHKKDLMQLILEGAMRSCDGNLWDKSAYRRFVVDNCKSIYFAGHDSTAVSVSWCLMLLALNPSWQVKIRDEILSSCKNGIPDAESIPNLKTVTMVIQETMRLYPPAPIVGREASKDIRLGDLVVPKGVCIWTLIPALHRDPEIWGPDANDFKPERFSEGISKACKYPQSYIPFGLGPRTCVGKNFGMMEVKVLVSLIVSKFSFTLSPTYQHSPSHKLLVEPQHGVVIRVV

SEQ ID NO: 30 - protein, 710 aa, Stevia rebaudiana
MQSDSVKVSPFDLVSAAMNGKAMEKLNASESEDPTTLPALKMLVENRELLTLFTTSFAVLIGCLVFLMWRRSSSKKLVQDPVPQVIVVKKKEKESEVDDGKKKVSIFYGTQTGTAEGFAKALVEEAKVRYEKTSFKVIDLDDYAADDDEYEEKLKKESLAFFFLATYGDGEPTDNAANFYKWFTEGDDKGEWLKKLQYGVFGLGNRQYEHFNKIAIVVDDKLTEMGAKRLVPVGLGDDDQCIEDDFTAWKELVWPELDQLLRDEDDTSVTTPYTAAVLEYRVVYHDKPADSYAEDQTHTNGHVVHDAQHPSRSNVAFKKELHTSQSDRSCTHLEFDISHTGLSYETGDHVGVYSENLSEVVDEALKLLGLSPDTYFSVHADKEDGTPIGGASLPPPFPPCTLRDALTRYADVLSSPKKVALLALAAHASDPSEADRLKFLASPAGKDEYAQWIVANQRSLLEVMQSFPSAKPPLGVFFAAVAPRLQPRYYSISSSPKMSPNRIHVTCALVYETTPAGRIHRGLCSTWMKNAVPLTESPDCSQASIFVRTSNFRLPVDPKVPVIMIGPGTGLAPFRGFLQERLALKESGTELGSSIFFFGCRNRKVDFIYEDELNNFVETGALSELIVAFSREGTAKEYVQHKMSQKASDIWKLLSEGAYLYVCGDAKGMAKDVHRTLHTIVQEQGSLDSSKAELYVKNLQMSGRYLRDVW

SEQ ID NO: 31 - protein, 692 aa, Arabidopsis thaliana
MTSALYASDLFKQLKSIMGTDSLSDDVVLVIATTSLALVAGFVVLLWKKTTADRSGELKPLMIPKSLMAKDEDDDLDLGSGKTRVSIFFGTQTGTAEGFAKALSEEIKARYEKAAVKVIDLDDYAADDDQYEEKLKKETLAFFCVATYGDGEPTDNAARFSKWFTEENERDIKLQQLAYGVFALGNRQYEHFNKIGIVLDEELCKKGAKRLIEVGLGDDDQSIEDDFNAWKESLWSELDKLLKDEDDKSVATPYTAVIPEYRVVTHDPRFTTQKSMESNVANGNTTIDIHHPCRVDVAVQKELHTHESDRSCIHLEFDISRTGITYETGDHVGVYAENHVEIVEEAGKLLGHSLDLVFSIHADKEDGSPLESAVPPPFPGPCTLGTGLARYADLLNPPRKSALVALAAYATEPSEAEKLKHLTSPDGKDEYSQWIVASQRSLLEVMAAFPSAKPPLGVFFAAIAPRLQPRYYSISSCQDWAPSRVHVTSALVYGPTPTGRIHKGVCSTWMKNAVPAEKSHECSGAPIFIRASNFKLPSNPSTPIVMVGPGTGLAPFRGFLQERMALKEDGEELGSSLLFFGCRNRQMDFIYEDELNNFVDQGVISELIMAFSREGAQKEYVQHKMMEKAAQVWDLIKEEGYLYVCGDAKGMARDVHRTLHTIVQEQEGVSSSEAEAIVKKLQTEGRYLRDVW

SEQ ID NO: 32 - protein, 524 aa, synthetic construct
MAHALAENILTELNTLLSDMDDGGYVGPSVYDTAQLLRFHPNPPDRAGIYRWLIKQQHEDGGWGSPDFPLHRQVPTVAAILALHEAQPQPEGAAAALAAAAVYLAQERDLYADTIPDDAPIGAELILPQLCRQAAALFPHLAYPRYGALYEAEAARLGKVESLTAVPSGHPLLHSWESWGRSSTEVTPDVFGSIGISPSATAVWLGRACAENPACLPEHATRYLHNASRATGVGIDGVVPNVWPIDVFEPCWSLYSLHLAGLFSHPGLSTVVQNIATNIQAILTPLGLGPALSFASDADDTAIAAAVVQLSGHSLTCYPLHQFEKGDLFVTFPGERNPSLSTTIHAVHALSLLGTTAPDARAYIENSKSADGVWKNEKWHASWLYPTSHAVAALAHGMPSWRDNDVLYKILEAQHLSGGWGAGAAPTQEETAYALFALHVMNDRVNAPLREKLVSAVARAREWLLVRYQSNQLPITPLWIGKELYCPQRVVRVTELTGLWLALNWNPSHSDVSDTRTETPGERI

SEQ ID NO: 33 - protein, 317 aa, synthetic construct
MATSHDDACQQVKVWGETLFGFLDEHAVEAVRGGQFILRHIRPELAAISARTGRDPDDEARELAFYQEMALLFWIDDCHDRGVMSPDDYAVVEGILVGRMPDAPTPSVGCSFLRHRLAQLASHKHDYSQLLADTQAYSTALRNGKRLASDPDRWSYSEHLRNGVDSIGYQNVFGCLSLLWGLDMPRWRTEPAFQNALSFLCAIGRLQNDLHGLANDRTLGEADNLAVQLERRYPTLDAVEFLQTEITGYERMLRPLLETANFDPVWVRLMETMLTVSDQYYATSTLRYRIDDTATTAPSCDTRHASGAVTGSGNETE

SEQ ID NO: 34 - protein, 517 aa, synthetic construct
MANALSEQILFELRHLLSEMSDGGSVGPSVYDTARALQFGGNVTGRQDAYAWLLAQQQADGGWGSADFPLFRHAPTWAALLALQRADPLPGAADAVQAATRFLERQADPYAHAVPEDAPIGAELILPQLCGEAASLLGGVAFPRHPALLPLRQACLVKLGAVATLPSGHPLLHSWEAWGTWPTAACPDDDGSIGISPAATAAWRAHAVTQGSTPQVGRADAYLQAASRATRSGIEGVVPNVWPINVFEPCWSLYTLHLAGLFAHPALDEAVRVIVAQLDARLGVRGLGPALHFAADADDTAVALCVLRLAGRDPAVDALRHFEIGELFVTFPGERNASVSTNIHALHALRLLGKPAAGTSAYVEANRNPHGLWDNEKWHVSWLYPTAHAVAALAQGKPQWRDERALAALLQAQRDDGGWGAGRASTFEETAYALFALHVMDGSEEPTGRRRIAQAVARALEWMLARHAAPALPQMPLWIGKELYCPIRVVRVAELAGLWLALRWGPRVPAEGAGAAP

SEQ ID NO: 35 - protein, 302 aa, synthetic construct
MAIPTERGLQQVLEWGRSLTGFADEHAAEAVRGGQYILQRIHPSLRDTSARTGRDPQDETLIVAFYRELALLFWLDDCNDLDLIAPEQLAAVEQALGQGVPCALPGFEGCAVLRASLAALAYDRRDYAQLLDDTRCYCAALRAGHAQAAGAAERWSYAEYLHNGIDSIAYANVFCCLSLLWGLDMATLRARPAFRQVLRLISAIGRLQNDLHGRDKDRSAGEADNAAILLLERYPAMPVVEFLNDELAGHTRMLHRVMAEERFPAPWGPLIEAMAAIRAHYYQTSTSRYRSDAAGGGQHAPA

SEQ ID NO: 36 - protein, 295 aa, synthetic construct
MAENGLLDCEQYLEEAMAEHATAQCPPLLAQALNYAVFPGGARVRPKICKAVALANNSSDVGLANAAASAIELLHCASLVHDDLPCFDDATQRRGKPSVHAKFGERIAVLTGDALIVAAFQTLATHAIHAVRTERVPLVTAIVARGVGAPHGICAGQAWECERSVDLSRYHRAKTGALFVAATCAGAAAAGVDPGPWVNLGASIGEAYQVADDIKDAISDPETLGKPTGIDVKLDRPSAVRELGLDGAVTRLKQCLEAGLDSMPACAGQDLLQKIVRAQASRFVPEKIAQVAAVD

SEQ ID NO: 37 - protein, 296 aa, Paracoccus haeundaensis
MARRDVNPIHATLLQTRLEEIAQGFGAVSQPLGAAMSHGALSSGRRFRGMLMLLAAEASGGVCDTIVDAACAVEMVHAASLIFDDLPCMDDAGLRRGRPATHVAHGESRAVLGGIALITEAMALLAGARGASGTVRAQLVRILSRSLGPQGLCAGQDLDLHAAKNGAGVEQEQDLKTGVLFIAGLEMLAVIKEFDAEEQTQMIDFGRQLGRVFQSYDDLLDVVGDQAALGKDTGRDAAAPGPRRGLLAVSDLQNVSRHYEASRAQLDAMLRSKRLQAPEIAALLERVLPYAARAVD

SEQ ID NO: 38 - protein, 335 aa, synthetic construct
MASSPITQAQVESKYRQYHAKINEALAACFPKEKPATLYDPARYILEGKGKRIRPFLTLLAAEAVSGKSDNALGVALGIEVLHNFTLMHDDIMDQADLRHGRPTVHKQWNVNAAILSGDMMIAYAYELALKAISSRHAEIIHIFNDANITICEGQALDMELEQRKDVTIADYLDMISKKTGRLISAALEAGGVAGDGTPEQIAALVTFGEKIGRAFQIQDDYLDIMAGDGKSGKVPGGDVINGKKTWLLLRSLELAEGADRELLQSIFDNNGTSPDNVPAVKAIFEKCGVLNETRAKINEDTEAALAALDALPFEEGRGYLRGFANILMKRDFVD

SEQ ID NO: 39 - protein, 300 aa, synthetic construct
MAVAQTFNLDTYLSQRQQQVEEALSAALVPAYPERIYEAMRYSLLAGGKRLRPILCLAACELAGGSVEQAMPTACALEMIHTMSLIHDDLPAMDNDDFRRGKPTNHKVFGEDIAILAGDALLAYAFEHIASQTRGVPPQLVLQVIARIGHAVAATGLVGGQVVDLESEGKAISLETLEYIHSHKTGALLEASVVSGGILAGADEELLARLSHYARDIGLAFQIVDDILDVTATSEQLGKTAGKDQAAAKATYPSLLGLEASRQKAEELIQSAKEALRPYGSQAEPLLALADFITRRQHVD

SEQ ID NO: 40 - protein, 305 aa, synthetic construct
MAVAQQTRTDFDLAQYLQVKKGVVEAALDSSLAIARPEKIYEAMRYSLLAGGKRLRPILCITACELCGGDEALALPTACALEMIHTMSLIHDDLPSMDNDDFRRGKPTNHKVYGEDIAILAGDGLLAYAFEYVVTHTPQADPQALLQVIARLGRTVGAAGLVGGQVLDLESEGRTDITPETLTFIHTHKTGALLEASVLTGAILAGATGEQQQRLARYAQNIGLAFQVVDDILDITATQEELGKTAGKDVKAQKATYPSLLGLEASRAQAQSLIDQAIVALEPFGPSAEPLQAIAEYIVARKYVD

SEQ ID NO: 41 - protein, 275 aa, synthetic construct
MAKKEKVEERIREILRPGWDLLTEEAMLYSATVGGKRIRPLLVLTLGEDLGVEEEKLLDVAVAVELFHTASLIHDDLPPIDNADFRRGKPSCHRTYGEDIALLAGDGLFFLAFSQISKIGNSKIFEEFSETAYKLLLGEAMDVEFERRKMEVSQEMVERMYAFKTGALFAFCFSAPFILKGKDHTKMKLLGEKFGVAFQIYDDLKDILGSFEKVGKDLGKDTEKVTLVKKVGIQKAREMADKYYEEVLKGIESEGLFRTLFLLKELKQMVEERVD

SEQ ID NO: 42 - protein, 374 aa, synthetic construct
MAKDVSLSSFDAHDLDLDKFPEVVRDRLTQFLDAQELTIADIGAPVTDAVAHLRSFVLNGGKRIRPLYAWAGFLAAQGHKNSSEKLESVLDAAASLEFIQACALIHDDIIDSSDTRRGAPTVHRAVEADHRANNFEGDPEHFGVSVSILAGDMALVWAEDMLQDSGLSAEALARTRDAWRGMRTEVIGGQLLDIYLESHANESVELADSVNRFKTAAYTIARPLHLGASIAGGSPQLIDALLHYGHDIGIAFQLRDDLLGVFGDPAITGKPAGDDIREGKRTVLLALALQRADKQSPEAATAIRAGVGKVTSPEDIAVITEHIRATGAEEEVEQRISQLTESGLAHLDDVDIPDEVRAQLRALAIRSTERRMVD

SEQ ID NO: 43 - protein, 333 aa, synthetic construct
MAVPAPEAIRQALQERLLARLDHPDPLYRDLLQDYPRRGGKMLRGLLTVYSALAHGAPLEAGLEAATALELFQNWVLVHDDIEDGSEERRGRPALHRLHPMPLALNAGDAMHAEMWGLLAEGLARGLFPPEVLLEFHEVVRRTAYGQHLDLLWTLGGTFDLRPEDYFRMVAHKAAYYTAVAPLRLGALLAGKTPPAAYEEGGLRLGTAFQIVDDVLNLEGGEAYGKERAGDLYEGKRTLILLRFLEEAPPEERARALALLALPREAKPEAEVGWLLERLLASRALAWAKAEAKRLQAEGLALLEAAFQDLPGKEALDHLRGLLAALVERRAVD

SEQ ID NO: 44 - protein, 335 aa, synthetic construct
MADVVSRLHQKYGAEVEKALVRYLSIGLAEDFREAVLYQVKTGGKRLRPLLTLAAAEAVSGQWRPALPAAAIVELIHNYSLIYDDIIDRGDVRRGLPTVRKAFGDNAAILVGIWYREAIEEAVLDTPKPTLFAKEVAEVIKAIDEGERLDILFEAAGRSDPYFVQARWREVTLDDYIKMVSLKTGALIAAAAKWGVLSVSDDRGLAEAAWNFGMAAGVAFQIIDDVLDIYGDPKKFGKEIGKDIKEHKRGNAVVAVALSHLGEGERRRLLEILAREVVEEADVREAVALLDSVGAREEALRLAARYREEAERHLAKIPNNGTLKELLDFIVAREY

SEQ ID NO: 45 - protein, 463 aa, synthetic construct
MATKGSSGMSLAERFWLTLSRSSLVVGRSCVEFEPETVPLLSTLRGKPITFLGLMPPLHEGRREDGEDATVRWLDAQPAKSVVYVALGSEVPLGVEKVHELALGLELAGTRFLWALRKPTGVSDADLLPAGFEERTRGRGVVATRWVPQMSILAHAAVGAFLTHCGWNSTIEGLMFGHPLIMLPIFGDQGPNARLIEAKNAGLQVARNDGDGSFDREGVAAAIRAVAVEEESSKVFQAKAKKLQEIVADMACHERYIDGFIQQLRSYKDDSGYSSSYAAAAGMHVVICPWLAFGHLLPCLDLAQRLASRGHRVSFVSTPRNISRLPPVRPALAPLVAFVALPLPRVEGLPDGAESTNDVPHDRPDMVELHRRAFDGLAAPFSEFLGTACADWVIVDVFHHWAAAAALEHKVPCAMMLLGSAEMIASIADERLEHAETESPAAAGQGRPAAAPTFEVARMKLIR

SEQ ID NO: 46 - protein, 463 aa, synthetic construct
MANHHECMNWLDDKPKESVVYVAFGSLVKHGPEQVEEITRALIDSDVNFLWVIKHKEEGKLPENLSEVIKTGKGLIVAWCKQLDVLAHESVGCFVTHCGFNSTLEAISLGVPVVAMPQFSDQTTNAKLLDEILGVGVRVKADENGIVRRGNLASCIKMIMEEERGVIIRKNAVKWKDLAKVAVHEGGSSDNDIVEFVSELIKAGSGEQQKIKKSPHVLLIPFPLQGHINPFIQFGKRLISKGVKTTLVTTIHTLNSTLNHSNTTTTSIEIQAISDGCDEGGFMSAGESYLETFKQVGSKSLADLIKKLQSEGTTIDAIIYDSMTEWVLDVAIEFGIDGGSFFTQACVVNSLYYHVHKGLISLPLGETVSVPGFPVLQRWETPLILQNHEQIQSPWSQMLFGQFANIDQARWVFTNSFYKLEEEVIEWTRKIWNLKVIGPTLPSMYLDKRLDDDKDNGFNLYKA

SEQ ID NO: 47 - protein, 8 aa, synthetic construct
MALLLAVF

SEQ ID NO: 48 - protein, 17 aa, synthetic construct
YKDDSGYSSSYAAAAGM

SEQ ID NO: 49 - protein, 7 aa, synthetic construct
YKDAAGM

SEQ ID NO: 50 - protein, 5 aa, synthetic construct
YGSGM

SEQ ID NO: 51 - protein, 482 aa, synthetic construct
MADAMATTEKKPHVIFIPFPAQSHIKAMLKLAQLLHHKGLQITFVNTDFIHNQFLESSGPHCLDGAPGFRFETIPDGVSHSPEASIPIRESLLRSIETNFLDRFIDLVTKLPDPPTCIISDGFLSVFTIDAAKKLGIPVMMYWTLAACGFMGFYHIHSLIEKGFAPLKDASYLTNGYLDTVIDWVPGMEGIRLKDFPLDWSTDLNDKVLMFTTEATQRSHKVSHHIFHTFDELEPSIIKTLSLRYNHIYTIGPLQLLLDQIPEEKKQTGITSLHGYSLVKEEPECFQWLQSKEPNSVVYVNFGSTTVMSLEDMTEFGWGLANSNHYFLWIIRSNLVIGENAVLPPELEEHIKKRGFIASWCSQEKVLKHPSVGGFLTHCGWGSTIESLSAGVPMICWPYSWDQLTNCRYICKEWEVGLEMGTKVKRDEVKRLVQELMGEGGHKMRNKAKDWKEKARIAIAPNGSSSLNIDKMVKEITVLARN

SEQ ID NO: 52 - skipped

SEQ ID NO: 53 - protein, 57 aa, synthetic construct
MDAVTGLLTVPATAITIGGTAVALAVALIFWYLKSYTSARRSQSNHLPRVPEVPGVP

SEQ ID NO: 54 - protein, 61 aa, synthetic construct
MALLLAVFTGLLTVPATAITIGGTAVALAVALIFWYLKSYTSARRSQSNHLPRVPEVPGVP

SEQ ID NO: 55 - protein, 37 aa, synthetic construct
AVALAVALIFWYLKSYTSARRSQSNHLPRVPEVPGVP

SEQ ID NO: 56 - protein, 18 aa, synthetic construct
RRSQSNHLPRVPEVPGVP

SEQ ID NO: 57 - protein, 18 aa, synthetic construct
RRSQSNHLPRVPEVPGVP

SEQ ID NO: 58 - protein, 480 aa, synthetic construct
MEESKTPHVAIIPSPGMGHLIPLVEFAKRLVHLHGLTVTFVIAGEGPPSKAQRTVLDSLPSSISSVFLPPVDLTDLSSSTRIESRISLTVTRSNPELRKVFDSFVEGGRLPTALVVDLFGTDAFDVAVEFHVPPYIFYPTTANVLSFFLHLPKLDETVSCEFRELTEPLMLPGCVPVAGKDFLDPAQDRKDDAYKWLLHNTKRYKEAEGILVNTFFELEPNAIKALQEPGLDKPPVYPVGPLVNIGKQEAKQTEESECLKWLDNQPLGSVLYVSFGSGGTLTCEQLNELALGLADSEQRFLWVIRSPSGIANSSYFDSHSQTDPLTFLPPGFLERTKKRGFVIPFWAPQAQVLAHPSTGGFLTHCGWNSTLESVVSGIPLIAWPLYAEQKMNAVLLSEDIRAALRPRAGDDGLVRREEVARVVKGLMEGEEGKGVRNKMKELKEAACRVLKDDGTSTKALSLVALKWKAHKKELEQNGNH

SEQ ID NO: 59 - protein, 465 aa, synthetic construct
MSMSDINKNSELIFIPAPGIGHLASALEFAKLLTNHDKNLYITVFCIKFPGMPFADSYIKSVLASQPQIQLIDLPEVEPPPQELLKSPEFYILTFLESLIPHVKATIKTILSNKVVGLVLDFFCVSMIDVGNEFGIPSYLFLTSNVGFLSLMLSLKNRQIEEVFDDSDRDHQLLNIPGISNQVPSNVLPDACFNKDGGYIAYYKLAERFRDTKGIIVNTFSDLEQSSIDALYDHDEKIPPIYAVGPLLDLKGQPNPKLDQAQHDLILKWLDEQPDKSVVFLCFGSMGVSFGPSQIREIALGLKHSGVRFLWSNSAEKKVFPEGFLEWMELEGKGMICGWAPQVEVLAHKAIGGFVSHCGWNSILESMWFGVPILTWPIYAEQQLNAFRLVKEWGVGLGLRVDYRKGSDVVAAEEIEKGLKDLMDKDSIVHKKVQEMKEMSRNAVVDGGSSLISVGKLIDDITGSN

SEQ ID NO: 60 - protein, 456 aa, synthetic construct
MSQTTTNPHVAVLAFPFSTHAAPLLAVVRRLAAAAPHAVFSFFSTSQSNASIFHDSMHTMQCNIKSYDISDGVPEGYVFAGRPQEDIELFTRAAPESFRQGMVMAVAETGRPVSCLVADAFIWFAADMAAEMGVAWLPFWTAGPNSLSTHVYIDEIREKIGVSGIQGREDELLNFIPGMSKVRFRDLQEGIVFGNLNSLFSRMLHRMGQVLPKATAVFINSFEELDDSLTNDLKSKLKTYLNIGPFNLITPPPVVPNTTGCLQWLKERKPTSVVYISFGTVTTPPPAEVVALSEALEASRVPFIWSLRDKARVHLPEGFLEKTRGYGMVVPWAPQAEVLAHEAVGAFVTHCGWNSLWESVAGGVPLICRPFFGDQRLNGRMVEDVLEIGVRIEGGVFTKSGLMSCFDQILSQEKGKKLRENLRALRETADRAVGPKGSSTENFITLVDLVSKPKDV

SEQ ID NO: 61 - protein, 482 aa, synthetic construct
MGNFANRKPHVVMIPYPVQGHINPLFKLAKLLHLRGFHITFVNTEYNHKRLLKSRGPKAFDGFTDFNFESIPDGLTPMEGDGDVSQDVPTLCQSVRKNFLKPYCELLTRLNHSTNVPPVTCLVSDCCMSFTIQAAEEFELPNVLYFSSSACSLLNVMHFRSFVERGIIPFKDESYLTNGCLETKVDWIPGLKNFRLKDIVDFIRTTNPNDIMLEFFIEVADRVNKDTTILLNTFNELESDVINALSSTIPSIYPIGPLPSLLKQTPQIHQLDSLDSNLWKEDTECLDWLESKEPGSVVYVNFGSTTVMTPEQLLEFAWGLANCKKSFLWIIRPDLVIGGSVIFSSEFTNEIADRGLIASWCPQDKVLNHPSIGGFLTHCGWNSTTESICAGVPMLCWPFFADQPTDCRFICNEWEIGMEIDTNVKREELAKLINEVIAGDKGKKMKQKAMELKKKAEENTRPGGCSYMNLNKVIKDVLLKQN

SEQ ID NO: 62 - protein, 454 aa, synthetic construct
MSTFKNEMNGNNLLHVAVLAFPFGTHAAPLLSLVKKIATEAPKVTFSFFCTTTTNDTLFSRSNEFLPNIKYYNVHDGLPKGYVSSGNPREPIFLFIKAMQENFKHVIDEAVAETGKNITCLVTDAFFWFGADLAEEMHAKWVPLWTAGPHSLLTHVYTDLIREKTGSKEVHDVKSIDVLPGFPELKASDLPEGVIKDIDVPFATMLHKMGLELPRANAVAINSFATIHPLIENELNSKFKLLLNVGPFNLTTPQRKVSDEHGCLEWLDQHENSSVVYISFGSVVTPPPHELTALAESLEECGFPFIWSFRGDPKEKLPKGFLERTKTKGKIVAWAPQVEILKHSSVGVFLTHSGWNSVLECIVGGVPMISRPFFGDQGLNTILTESVLEIGVGVDNGVLTKESIKKALELTMSSEKGGIMRQKIVKLKESAFKAVEQNGTSAMDFTTLIQIVTS

SEQ ID NO: 63 - protein, 446 aa, synthetic construct
MKNKQHVAIFPFPFGSHLPPLLNLVLKLAHIAPNTSFSFIGTHSSNAFLFTKRHIPNNIRVFTISDGIPEGHVPANNPIEKLDLFLSTGPDNLRKGIELAVAETKQSVTCIIADAFVTSSLLVAQTLNVPWIAFWPNVSCSLSLYFNIDLIRDKCSKDAKNATLDFLPGLSKLRVEDVPQDMLDVGEKETLFSRTLNSLGVVLPQAKAVVVNFFAELDPPLFVKYMRSKLQSLLYVVPLPCPQLLLPEIDSNGCLSWLDSKSSRSVAYVCFGTVVSPPPQEVVAVAEALEESGFPFVWALKESLLSILPKGFVERTSTRGKVVSWVPQSHVLSHGSVGVFVTHCGANSVMESVSNGVPMICRPFFGDQGIAARVIQDIWEVGVIVEGKVFTKNGFVKSLNLILVQEDGKKIRDNALKVKQIVQDAVGPHGQAAEDFNTLVEVISSS

SEQ ID NO: 64 - protein, 4 aa, synthetic construct
PSPG

SEQ ID NO: 65 - skipped